SEQUENCE LISTING (11 sequences)

SEQ ID NO: 1 - protein, 42 aa, Homo sapiens [sequence omitted]

SEQ ID NO: 2 - protein, 10 aa, synthetic construct
  REGION 1..10: note = Synthetic Construct
GFTFSSYGMS

SEQ ID NO: 3 - protein, 16 aa, synthetic construct
  REGION 1..16: note = Synthetic Construct
SINSNGGSTYYPDSVK

SEQ ID NO: 4 - skipped

SEQ ID NO: 5 - protein, 438 aa, synthetic construct
  REGION 1..438: note = Synthetic Construct
EVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 6 - protein, 16 aa, synthetic construct
  REGION 1..16: note = Synthetic Construct
RSSQSLVYSNGDTYLH

SEQ ID NO: 7 - protein, 7 aa, synthetic construct
  REGION 1..7: note = Synthetic Construct
KVSNRFS

SEQ ID NO: 8 - protein, 9 aa, synthetic construct
  REGION 1..9: note = Synthetic Construct
SQSTHVPWT

SEQ ID NO: 9 - protein, 219 aa, synthetic construct
  REGION 1..219: note = Synthetic Construct
DIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 112 aa, synthetic construct
  REGION 1..112: note = Synthetic Construct
EVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSS

SEQ ID NO: 11 - protein, 112 aa, synthetic construct
  REGION 1..112: note = Synthetic Construct
DIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIK

(19) United States
(12) Patent Application Publication
(10) Pub. No.: US 2025/0361292 A1
(43) Pub. Date: Nov. 27, 2025

(54) METHODS OF DELAYING OR PREVENTING THE ONSET OF ALZHEIMER'S DISEASE USING CRENEZUMAB

(71) Applicants: GENENTECH, INC., South San Francisco, CA (US); BANNER HEALTH, Phoenix, AZ (US)

(72) Inventors: Nan HU, Foster City, CA (US); Kaycee Michelle SINK, Lewisville, NC (US); Shehnaaz SULIMAN, South San Francisco, CA (US); Carole HO, South San Francisco, CA (US); Eric REIMAN, Phoenix, AZ (US); Pierre TARIOT, Phoenix, AZ (US); Jessica LANGBAUM, Phoenix, AZ (US)

(21) Appl. No.: 18/874,482

(22) PCT Filed: Jun. 13, 2023
(86) PCT No.: PCT/US2023/068359

§ 371 Date: Dec. 12, 2024

Related U.S. Application Data
(60) Provisional application No. 63/351,790, filed on Jun. 13, 2022.
(60) Provisional application No. 63/370,100, filed on Aug. 1, 2022.
(60) Provisional application No. 63/385,541, filed on Nov. 30, 2022.

(51) Int. Cl. C07K 16/18; A61P 25/28

(52) U.S. Cl. CPC C07K 16/18; A61P 25/28

(57) ABSTRACT

Provide herein are methods of treating human patients with familial Alzheimer's disease that result in delayed in symptom onset and/or slowed cognitive decline by administering a humanized monoclonal anti-amyloid beta (Aβ) antibody.

### CROSS-REFERENCE TO RELATED APPLICATIONS

This application claims the benefit of and priority to U.S. Provisional Application No. 63/351,790, filed Jun. 13, 2022, and U.S. Provisional Application No. 63/370,100, filed Aug. 1, 2022, and Provisional Application No. 63/385,541, filed Nov. 30, 2022, the disclosures of which are hereby incorporated herein by reference in their entireties.

### GOVERNMENT FUNDING

This invention was made with government support under, including but not limited to, RF1 AG041705 and R01 AG055444.

### SUBMISSION OF SEQUENCE LISTING ON ASCII TEXT FILE

The content of the following submission on XML file is incorporated herein by reference in its entirety: a computer readable form (CRF) of the Sequence Listing (file name: 146392062440seqlist.XML, date created: Jun. 12, 2023, size: (14,016 bytes).

### FIELD OF THE INVENTION

The present disclosure relates to methods of delaying onset of symptoms or slowing cognitive decline by administering crenezumab.

### BACKGROUND

Alzheimer's Disease (AD) is the most common cause of dementia, affecting an estimated 4.5 million individuals in the United States and 26.6 million worldwide (Hebert et al., Arch. Neurol. 2003; 60:1119-22; Brookmeyer et al., Alzheimers Dement. 2007; 3:186-91). The disease is characterized pathologically by the accumulation of extracellular β-amyloid (“AB”) plaques and intracellular neurofibrillary tangles in the brain. Diagnosis is made through the clinical assessment of the neurologic and neuropsychiatric signs and symptoms of AD and the exclusion of other causes of dementia. AD is commonly classified into stages based on cognitive screening examination tests, such as the Mini-Mental State Examination (“MMSE”) or other tests. Currently, there are no effective therapies that are approved and known to modify or prevent progression of the disease: Approved medical therapies, such as those that inhibit acetylcholinesterase (“AChE”) activity or antagonize N-methyl-D-aspartate receptors in the brain, may temporarily improve the symptoms of AD in some patients but do not modify or prevent the progression of the disease (Cummings, N. Engl. J. Med. 2004; 351:56-67). The deposition of extracellular amyloid plaques in the brain is a hallmark pathologic finding in AD, first reported by Alois Alzheimer in 1906. These amyloid plaques are primarily composed of amyloid beta (Aβ) peptides (Haass and Selkoe, Nat. Rev. Mol. Cell Biol. 2007, 8 (2): 101-112) generated by the sequential cleavage of amyloid precursor protein (“APP”) via β and γ-secretase activity. Techniques and tools have been developed to visualize the presence of plaques in patients. For example, positron emission tomography (“PET”) scans using imaging agents, such as 18F-florbetapir, that detect Aβ can be used to detect the presence of amyloid in the brain.

A number of genetic factors in early- and late-onset familial AD have been documented. The Presenilin 1 (PSEN1) allele is strongly associated with heritable autosomal-dominant AD (ADAD), with clinical onset occurring before 60 years of age and overproduction of Aβ-42 up to 20 years prior to becoming symptomatic. PET imaging in familial carriers of a PSEN1 E280A mutation provided striking evidence of cerebellar Aβ plaque deposition nearly a decade in advance of ADAD onset (Ghisays et al., NeuroImage: Clinical, 2021; 31:102749). Other genetic factors, such as APP and PSEN2, have also been identified and characterized.

Aβ, particularly in its oligomerized forms, is toxic to neurons and is believed to be causative in AD. Therapies that reduce Aβ levels in the brain may alleviate cognitive dysfunction and block further synaptic loss, axon degeneration, and neuronal cell death. Aβ can be transported actively across the blood-brain barrier (Deane et al., Stroke, 2004; 35 (Suppl I): 2628-31). In murine models of AD, systemic delivery of antibodies to Aβ increases Aβ levels in plasma while reducing levels in the central nervous system (CNS) through several proposed mechanisms, including dissolution of brain Aβ plaque, phagocytic removal of opsonized Aβ, and finally via efflux of Aβ from the brain as a result of an equilibrium shift of Aβ resulting from circulating antibodies (Morgan (2005), Neurodegener. Dis.; 2:261-6).

Significant failures have marked the development of therapeutic antibodies for the treatment of AD. Large-scale phase three clinical trials of bapincuzumab, an IgG1 isotype antibody binding specifically to the N-terminal portion of Aβ, were halted when administration of the drug failed to arrest cognitive decline in treated patients (Miles et al., Scientific Reports. 2013; 3:1-4 Johnston & Johnson press release dated Aug. 6, 2012, entitled “Johnson & Johnson Announces Discontinuation of Phase 3 Development of Bapineuzumab Intravenous (IV) in Mild-To-Moderate Alzheimer's Disease”). Notably, bapineuzumab did appear to stabilize plaque levels and decreased phosphorylated tau levels in cerebrospinal fluid-suggesting that modification of these biomarkers alone is not necessarily predictive of clinical efficacy (Miles et al., Scientific Reports, 2013; 3:1-4). Similarly, in phase three clinical trials of solanezumab, an antibody specific for monomeric Aβ that binds in the middle portion of the peptide, the primary cognitive and functional endpoints were not met (Eli Lilly and Company press release dated Aug. 24, 2012, “Eli Lilly and Company Announces Top-Line Results on Solanezumab Phase 3 Clinical Trials in Patients with Alzheimer's Disease”). Safety concerns have also been raised during the investigation of certain immunotherapies for AD: incidence of amyloid-related imaging abnormalities (ARIA-E and ARIA-H) was over 20% among drug-treated patients in phase two clinical trials of bapineuzumab (Sperling et al., The Lancet, 2012; 11:241-249). More recently, an IgG1 isotype anti-Aβ antibody binding to aggregated but not monomeric forms of amyloid β, aducanumab, was reported to trigger ARIA-E, a form of edema in the brain, in subjects enrolled in a Phase I clinical trial. In a multiple-ascending-dose trial, ARIA-E was detected in an increasing percentage of subjects as the dose was increased and the percentage of subjects with ARIA-E was increased when looking at the subset of subjects carrying an ApoE4 allele, a risk factor for AD. Reportedly, 5% of subjects dosed at 1 and 3 mg/kg of the anti-Aβ antibody showed ARIA-E but 43% and 55% of subjects dosed at 6 mg/kg and 10 mg/kg respectively exhibited ARIA-E. Thus, at increasing doses, the incidence of ARIA-E adverse events also increased. See Press Coverage of 2015 Alzheimer's Association International Conference reporting by Gabrielle Strobel, Part 4 of 15, accessible at: www.alzforum.org/news/conference-coverage/aducanumab-solanezumab-gantenerumab-data-lift-crenezumab-well (accessed Jan. 18, 2016). One third of the ARIA-E events led to symptoms in the subjects and some of the patients were reported to have discontinued or had their dose of anti-amyloid antibody reduced.

It is estimated that one in nine people over the age of 65 have AD; CDC estimates for 2020 indicating that 5.8 million people over the age of 65 have AD, which is projected to nearly triple to 14 million by 2060. The CDC identifies AD as the sixth-leading cause of adult death and fifth-leading cause of death in adults older than 65 in the United States. The aggregated yearly costs for health care, long-term care, and hospice care by and on behalf of individuals afflicted with AD are over $321 billion in 2022 and are estimated to rise to $1.2 trillion by 2050 (by and on behalf of affected individuals), not accounting for the collective 12 billion hours spent by 11 million unpaid caretakers that is valued at an additional $272 billion in 2021 (Alzheimer's Association 2022 Alzheimer's Disease Facts and Figures, Alzheimer's and Dementia 18). Current approved therapies treat only some of the symptoms of AD, and not the underlying degeneration. There is a tremendous unmet need for a safe and effective disease-modifying and disease-preventing therapeutic for AD.

### BRIEF SUMMARY

In one aspect, provided here is a method of delaying onset of at least one symptom in a human patient with a genetic mutation that causes familial Alzheimer's Disease (AD) comprising administering to the human patient an effective amount of a humanized monoclonal anti-amyloid beta (Aβ) antibody, wherein administering such treatment to a plurality of human patients results in a delayed onset of at least one symptom in the plurality of human patients compared to a reference onset of at least one symptom, wherein the reference onset of at least one symptom is of a plurality of human patients who have received a placebo, wherein the antibody comprises six hypervariable regions (HVRs), wherein (i) HVR-H1 comprises the amino acid sequence set forth in SEQ ID NO: 2; (ii) HVR-H2 comprises the amino acid sequence set forth in SEQ ID NO:3; (iii) HVR-H3 comprises the amino acid sequence set forth in SEQ ID NO:4; (iv) HVR-L1 comprises the amino acid sequence set forth in SEQ ID NO:6; (v) HVR-L2 comprises the amino acid sequence set forth in SEQ ID NO:7; and (vi) HVR-L3 comprises the amino acid sequence set forth in SEQ ID NO:8.

In another aspect, provided herein is a method of slowing cognitive decline in a human patient with a genetic mutation that causes familial Alzheimer's Disease (AD) comprising administering to the human patient an effective amount of a humanized monoclonal anti-amyloid beta (Aβ) antibody, wherein administering such treatment to a plurality of human patients results in delayed cognitive decline in the plurality of human patients compared to a reference cognitive decline, wherein the reference cognitive decline is of a plurality of human patients who have received a placebo, wherein the antibody comprises six hypervariable regions (HVRs), wherein (i) HVR-H1 comprises the amino acid sequence set forth in SEQ ID NO:2; (ii) HVR-H2 comprises the amino acid sequence set forth in SEQ ID NO:3; (iii) HVR-H3 comprises the amino acid sequence set forth in SEQ ID NO:4; (iv) HVR-L1 comprises the amino acid sequence set forth in SEQ ID NO:6; (v) HVR-L2 comprises the amino acid sequence set forth in SEQ ID NO: 7; and (vi) HVR-L3 comprises the amino acid sequence set forth in SEQ ID NO:8.

In still another aspect, provided herein is a method of preventing cognitive impairment in a human patient with a genetic mutation that causes familial Alzheimer's Disease (AD) comprising administering to the human patient an effective amount of a humanized monoclonal anti-amyloid beta (Aβ) antibody, wherein administering such treatment to a plurality of human patients results in reduced cognitive impairment in the plurality of human patients compared to a reference cognitive impairment, wherein the reference cognitive impairment is of a plurality of human patients who have received a placebo, wherein the antibody comprises six hypervariable regions (HVRs), wherein (i) HVR-H1 comprises the amino acid sequence set forth in SEQ ID NO:2; (ii) HVR-H2 comprises the amino acid sequence set forth in SEQ ID NO:3; (iii) HVR-H3 comprises the amino acid sequence set forth in SEQ ID NO:4; (iv) HVR-L1 comprises the amino acid sequence set forth in SEQ ID NO:6; (v) HVR-L2 comprises the amino acid sequence set forth in SEQ ID NO:7; and (vi) HVR-L3 comprises the amino acid sequence set forth in SEQ ID NO:8.

In some embodiments, administering such treatment results in a delay in onset of at least one symptom compared to the reference onset of at least one symptom after treatment of about five years or longer.

In some embodiments, administering such treatment results in a slowing of cognitive decline in the plurality of human patients compared to the reference cognitive decline after treatment of about five years or longer.

In some embodiments, administering such treatment results in a prevention or reduction of cognitive impairment in the plurality of human patients compared to the reference cognitive impairment after treatment of about five years or longer.

In some embodiments, the delay in onset of at least one symptom, the slowing in cognitive decline, or the prevention of cognitive impairment is measured using an API ADAD Cognitive Composite Test Battery, wherein, in the API ADAD Cognitive Composite Test Battery, administering such treatment to the plurality of human patients results in a reduced annualized rate of change on an API ADAD Composite score of the plurality of human patients relative to a reference annualized rate of change on an API ADAD Composite score, and wherein the reference annualized rate of change on an API ADAD Composite score is an annualized rate of change on an API ADAD Composite score of a plurality of human patients who have received the placebo. In some embodiments, the API ADAD Composite Cognitive Test Battery comprises a Word List Recall, Multilingual Naming Test, Mini-Mental State Examination (MMSE), CERAD Constructional praxis, and Raven's Progressive Matrices. In some embodiments, administering such treatment results in a reduced annualized rate of change in the API ADAD Composite score after treatment of about five years or longer. In some embodiments, administering such treatment results in a reduction of the annualized rate of change on the API ADAD Composite score in the plurality of human patients by at least 20% relative to the reference annualized rate of change on the API ADAD Composite score. In some embodiments, administering such treatment results in a reduction of the annualized rate of change in the API ADAD Composite score in the plurality of human patients by at least 30% relative to the reference annualized rate of change on the API ADAD Composite score. In some embodiments, administering such treatment results in a reduction of the annualized rate of change in the API ADAD Composite score in the plurality of human patients by 20% to 40% relative to the reference annualized rate of change on the API ADAD Composite score.

In some embodiments, administering such treatment to the plurality of human patients results in a reduced annualized rate of change on a Free and Cued Selective Reminding Task (FCSRT) Cueing Index of the plurality of human patients relative to a reference annualized rate of change on a FCSRT Cueing Index, wherein the reference annualized rate of change on the FCSRT Cueing Index is an annualized rate of change on a FCSRT Cueing Index of a plurality of human patients who have received the placebo. In some embodiments, administering such treatment results in a reduced annualized rate of change on the FCSRT Cueing Index in the plurality of human patients compared to the reference annualized rate of change on the FCSRT Cueing Index after treatment of about five years or longer. In some embodiments, administering such treatment results in a reduction of the annualized rate of change of the FCSRT Cueing Index in the plurality of human patients by at least 10% relative to the reference annualized rate of change on the FCSRT Cueing Index. In some embodiments, administering such treatment results in a reduction of the annualized rate of change of the FCSRT Cueing Index in the plurality of human patients by at least 20% relative to the reference annualized rate of change on the FCSRT Cueing Index. In some embodiments, administering such treatment results in a reduction of the annualized rate of change of the FCSRT Cueing Index in the plurality of human patients by 10% to about 30% relative to the reference annualized rate of change on the FCSRT Cueing Index. In some embodiments, the FCSRT Cueing Index is assessed using controlled learning.

In some embodiments, administering such treatment to the plurality of human patients results in an increased time to progression from preclinical AD to mild cognitive impairment due to AD or from preclinical AD to dementia due to AD in the plurality of human patients relative to a reference time to progression from preclinical AD to mild cognitive impairment due to AD or from preclinical AD to dementia due to AD, wherein the reference time to progression from preclinical AD to mild cognitive impairment due to AD or from preclinical AD to dementia due to AD is a time to progression of a plurality of human patients who have received the placebo. In some embodiments, administering such treatment results in an increased time to progression from preclinical AD to mild cognitive impairment due to AD or from preclinical AD to dementia due to AD in the plurality of human patients relative to the reference time to progression from preclinical AD to mild cognitive impairment due to AD or from preclinical AD to dementia due to AD after treatment of about five years or longer. In some embodiments, administering such treatment results in an increase of the time to progression from preclinical AD to mild cognitive impairment due to AD or from preclinical AD to dementia due to AD in the plurality of human patients by at least 10% as compared to a reference time to progression from preclinical AD to mild cognitive impairment due to AD or from preclinical AD to dementia due to AD. In some embodiments, administering such treatment results in an increase of the time to progression from preclinical AD to mild cognitive impairment due to AD or from preclinical AD to dementia due to AD in the plurality of human patients by at least 20% relative to a reference time to progression from preclinical AD to mild cognitive impairment due to AD or from preclinical AD to dementia due to AD. In some embodiments, administering such treatment results in an increase of the time to progression from preclinical AD to mild cognitive impairment due to AD or from preclinical AD to dementia due to AD in the plurality of human patients by 10% to 30% relative to a reference time to progression from preclinical AD to mild cognitive impairment due to AD or from preclinical AD to dementia due to AD.

In some embodiments, administering such treatment to the plurality of human patients results in an increased time to progression to non-zero in the Clinical Dementia Rating (CDR) Scale global score of the plurality of human patients relative to a reference time to progression to non-zero in the CDR Scale global score, wherein the reference time to progression to non-zero in the CDR Scale global score is a time to progression of a plurality of human patients who have received the placebo. In some embodiments, the CDR Scale global score describes impairment in memory, orientation, judgment and problem solving, community affairs, home and hobbies, and personal care. In some embodiments, administering such treatment results in an increased time to progression to non-zero in the CDR Scale global score in the plurality of human patients by 5% relative to a reference time to progression to progression to non-zero in the CDR Scale global score. In some embodiments, administering such treatment results in an increased time to progression to non-zero in the CDR Scale global score in the plurality of human patients by 10% relative to a reference time to progression to progression to non-zero in the CDR Scale global score. In some embodiments, administering such treatment results in an increased time to progression to non-zero in the CDR Scale global score in the plurality of human patients by 5% to 20% relative to a reference time to progression to progression to non-zero in the CDR Scale global score.

In some embodiments, administering such treatment to the plurality of human patients results in a reduced annualized rate of change on a Clinical Dementia Rating (CDR) Scale Sum of Boxes of the plurality of human patients relative to a reference annualized rate of change on a CDR Scale Sum of Boxes, wherein the reference annualized rate of change on a CDR Scale Sum of Boxes is an annualized rate of change on a CDR Scale Sum of Boxes of a plurality of human patients who have received placebo. In some embodiments, administering such treatment results in a reduced annualized rate of change on a CDR Scale Sum of Boxes of the plurality of human subjects compared to the reference annualized rate of change on a CDR Scale Sum of Boxes after treatment of about five years or longer. In some embodiments, administering such treatment results in a reduced annualized rate of change in a CDR Scale Sum of Boxes global score in the plurality of human patients by at least 5% relative to a reference CDR Scale Sum of Boxes global score. In some embodiments, administering such treatment results in a reduced annualized rate of change in a CDR Scale Sum of Boxes global score in the plurality of human patients by at least 10% relative to a reference CDR Scale Sum of Boxes global score. In some embodiments, administering such treatment results in a reduced annualized rate of change in a CDR Scale Sum of Boxes global score in the plurality of human patients by 5% to 20% relative to a reference CDR Scale Sum of Boxes global score.

In some embodiments, administering such treatment to the plurality of human patients results in a reduced annualized rate of change in a measure of overall neurocognitive functioning of the plurality of human patients relative to a reference annualized rate of change in a measure of overall neurocognitive functioning wherein the reference annualized rate of change in a measure of overall neurocognitive functioning is an annualized rate of change in a measure of overall neurocognitive functioning of the plurality of human patients who have received placebo. In some embodiments, the annualized rate of change in a measure of overall neurocognitive functioning is determined using a Repeatable Battery for the Assessment of Neuropsychological Status (RBANS) score. In some embodiments, administering such treatment results in a reduction of an annualized rate of change of a RBANS score in the plurality of human patients compared to a reference annualized rate of change of a RBANS score, wherein the reference annualized rate of change of a RBANS score is an annualized rate of change of a RBANS score of a plurality of human patients who have received placebo. In some embodiments, administering such treatment results in a reduction of the annualized rate of change of a RBANS score in the plurality of human patients compared to the reference annualized rate of change of a RBANS score after treatment of about 5 years or longer. In some embodiments, administering such treatment results in a reduction of the RBANS score by at least 30% relative to the reference RBANS score. In some embodiments, administering such treatment results in a reduction of the RBANS score by at least 40% relative to the reference RBANS score. In some embodiments, administering such treatment results in a reduction of the RBANS score by 30% to 50% relative to the reference RBANS score.

In some embodiments, administering such treatment to the plurality of human patients results in an effect on a tau-based CSF biomarker compared to a reference tau-based CSF biomarker, wherein the reference tau-based CSF biomarker is the tau-based CSF biomarker of a plurality of human patients who have received the placebo. In some embodiments, the tau-based CSF biomarker is measured using positron emission tomography. In some embodiments, administering such treatment results in a reduction of annualized rate of change in the tau-based CSF biomarker in the plurality of human patients compared to a reference annualized rate of change in the tau-based CSF biomarker, wherein the reference tau-based CSF biomarker is an annualized rate of change in the tau-based CSF biomarker of a plurality of human patients who have received the placebo. In some embodiments, administering such treatment results in a reduction of annualized rate of the tau-based CSF biomarker in the plurality of human patients by at least 30% relative to the reference tau-based CSF biomarker, said tau-based CSF biomarker being a phospho-tau [ptau]-based CSF biomarker. In some embodiments, wherein administering such treatment results in a reduction of annualized rate of the tau-based CSF biomarker in the plurality of human patients by 30% to 50% relative to the reference tau-based CSF biomarker, said tau-based CSF biomarker being a phospho-tau [ptau]-based CSF biomarker. In some embodiments, administering such treatment results in a reduction of annualized rate of the tau-based CSF biomarker in the plurality of human patients by at least 20% relative to the reference tau-based CSF biomarker, said tau-based CSF biomarker being a total-tau [ttau]-based CSF biomarker. In some embodiments, administering such treatment results in a reduction of annualized rate of the tau-based CSF biomarker in the plurality of human patients by 20% to 40% relative to the reference tau-based CSF biomarker, said tau-based CSF biomarker being a total-tau [ttau]-based CSF biomarker.

In some embodiments, administering such treatment to the plurality of human patients results in an effect on a brain tau load compared to a reference a brain tau load, wherein the reference brain tau load is a brain tau load of a plurality of human patients who have received the placebo. In some embodiments, the brain tau load is measured using positron emission tomography (tau-PET). In some embodiments, administering such treatment results in a reduction of annualized rate of change in a tau-PET measurement in the plurality of human patients relative to a reference annualized rate of change in a tau-PET measurement, wherein the reference annualized rate of change in a tau-PET measurement is an annualized rate of change in the tau-PET measurement of a plurality of human patients who have received the placebo. In some embodiments, administering such treatment results in a reduced Standardized Uptake Value Ratio (SUVR) of entorhinal cortex (ERC) tau-PET measurement in the plurality of human patients as compared to a reference SUVR of ERC tau-PET, wherein the reference SUVR of aERC tau-PET is a SUVR of ERC tau-PET of the plurality of human patients who have received the placebo. In some embodiments, the tau-PET is measured using Tau Probe 1, which is [¹⁸F]GTP1. In some embodiments, administering such treatment results in a reduction of annualized rate of the tau-PET in the plurality of human patients by at least 50% relative to the reference tau-PET. In some embodiments, administering such treatment results in a reduction of annualized rate of the tau-PET in the plurality of human patients by 40% to 60% relative to the reference tau-PET.

In some embodiments, administering such treatment to the plurality of human patients results in a reduction of cerebral fibrillary amyloid burden in a predefined region of interest of the plurality of human patients relative to a reference cerebral fibrillary amyloid burden in a predefined region of interest, wherein the reference cerebral fibrillary amyloid burden in a predefined region of interest is a cerebral fibrillary amyloid burden in a predefined region of interest of a plurality of human patients who have received the placebo. In some embodiments, the cerebral fibrillary amyloid burden is measured using florbetapir positron emission tomography (PET). In some embodiments, administering such treatment results in a reduction of annualized rate of change in amyloid burden in the plurality of human patients compared to a reference annualized rate of change in amyloid burden, wherein the reference annualized rate of change in amyloid burden is an annualized rate of change in amyloid burden of a plurality of human patients who have received the placebo. In some embodiments, administering such treatment results in a reduction of annualized rate of change in amyloid burden measured by PET in the plurality of human patients by 3% relative to a reference amyloid burden measured by PET. In some embodiments, administering such treatment results in a reduction of annualized rate of change in amyloid burden measured by PET in the plurality of human patients by 3% to 10% relative to a reference amyloid burden measured by PET. In some embodiments, administering such treatment to the plurality of human patients results in a reduced decline in regional cerebral metabolic rate of glucose (CMRgI) of the plurality of human patients relative to a reference CMRgI, wherein the reference CMRgI is a CMRgI of the plurality of human patients who have received the placebo. In some embodiments, the CMRgI is measured using FDG (fluorodeoxyglucose)-positron emission tomography (PET). In some embodiments, administering such treatment results in a reduced FDG PET measurement in the plurality of human patients relative to a reference FDG PET measurement, wherein the reference FDG PET measurement is a FDG PET measurement of the plurality of human patients who have received the placebo. In some embodiments, administering such treatment results in a reduced decline in regional CMRgI of the plurality of human patients compared to the reference CMRgI after treatment of about five years or longer. In some embodiments, administering such treatment results in a reduced annualized Standardized Uptake Value Ratio (SUVR) of FDG PET measurement in the plurality of human patients as compared to a reference annualized SUVR of FDG PET, wherein the reference annualized SUVR of FDG PET is a SUVR of FDG PET of the plurality of human patients who have received the placebo. In some embodiments, administering such treatment results in a reduced annualized Standardized Uptake Value Ratio (SUVR) of FDG PET measurement in the plurality of human patients by at least 10% as compared to a reference annualized SUVR of FDG PET. In some embodiments, administering such treatment results in a reduced annualized Standardized Uptake Value Ratio (SUVR) of FDG PET measurement in the plurality of human patients by 10% to 30% relative to a reference annualized SUVR of FDG PET.

In some embodiments, administering such treatment to the plurality of human patients results in a reduced brain atrophy of the plurality of human patients relative to a reference brain atrophy, wherein the reference brain atrophy is a brain atrophy of the plurality of human patients who have received the placebo. In some embodiments, administering such treatment results in a reduction of the brain atrophy of the plurality of human patients compared to the reference brain atrophy after treatment of about five years or longer.

In some embodiments, administering such treatment to the plurality of human patients results in a reduced annualized rate of change in the brain atrophy of the plurality of human patients relative to a reference annualized rate of change in the brain atrophy, wherein the reference annualized rate of change in the brain atrophy is an annualized rate of change in the brain atrophy of the plurality of human patients who have received the placebo. In some embodiments, administering such treatment results in a reduction of an annualized rate of change in the brain atrophy of the plurality of human patients compared to the reference annualized rate of change in the brain atrophy after treatment of about five years or longer. In some embodiments, the brain atrophy is measured using volumetric MRI. In some embodiments, the volumetric MRI is measured in a whole brain. In some embodiments, administering such treatment results in a reduction of an annualized rate of change in the brain atrophy of the plurality of human patients by at least 5% relative to the reference brain atrophy, said reduction being measured by a volumetric MRI on a whole brain. In some embodiments, administering such treatment results in a reduction of an annualized rate of change in the brain atrophy of the plurality of human patients by 5% to 20% relative to the reference brain atrophy, said reduction being measured by a volumetric MRI on a whole brain.

In some embodiments, the volumetric MRI is measured in a bilateral hippocampus. In some embodiments, administering such treatment results in a reduction of an annualized rate of change in the brain atrophy of the plurality of human patients by at least 1% relative to the reference brain atrophy, said reduction being measured by a volumetric MRI on a bilateral hippocampus. In some embodiments, administering such treatment results in a reduction of an annualized rate of change in the brain atrophy of the plurality of human patients by 1% to 10% relative to the reference brain atrophy, said reduction being measured by a volumetric MRI on a bilateral hippocampus.

In some embodiments, administering such treatment results in a reduction in change over baseline in a cognitive measurement of the plurality of human patients compared to a reference cognitive measurement, wherein the reference cognitive measurement is a cognitive measurement of a plurality of human patients who have received the placebo, wherein the cognitive measurement is selected from the group consisting of i) Trial Making Test, ii) Mini-Mental State Examination (MMSE), iii) Repeatable Battery for the Assessment of Neuropsychological Status (RBANS) Index Scores, iv) scores of each of the components of the API ADAD Composite Cognitive Test Battery, v) Preclinical Alzheimer's Cognitive Composite (PACC), and vi) other clinical endpoints. In some embodiments, administering such treatment results in a reduction in change over baseline in the cognitive measurement of the plurality of human patients compared to the reference cognitive measurement after treatment of about five years or longer.

In some embodiments, administering such treatment results in a statistically significant reduction in change over baseline in a Neuropsychiatric Inventory (NPI) of the plurality of human patients compared to a reference NPI, wherein the reference NPI is a NPI of a plurality of human patients who have received the placebo. In some embodiments, administering such treatment results in a statistically significant reduction in change over baseline in NPI after treatment of about five years or longer.

In some embodiments, administering such treatment results in a statistically significant reduction in change over baseline in a Geriatric Depression Scale (GDS) of the plurality of human patients compared to a reference GDS, wherein the reference GDS is a GDS of a plurality of human patients who have received the placebo. In some embodiments, administering such treatment results in a reduction in change over baseline in GDS after treatment of about five years or longer.

In some embodiments, administering such treatment results in a reduction in change over baseline in a Changes in Functional Assessment Staging of Alzheimer's Disease (FAST) total score of the plurality of human patients compared to a reference FAST total score, wherein the reference FAST total score is the FAST total score of a plurality of human patients who have received the placebo. In some embodiments, administering such treatment results in a reduction in change over baseline in FAST total score after treatment of about five years or longer.

In some embodiments, administering such treatment results in a reduction in change over baseline in a Changes in Subject Memory Checklist of the plurality of human patients compared to a reference Changes in Subject Memory Checklist, wherein the reference Changes in Subject Memory Checklist is a Changes in Subject Memory Checklist of a plurality of human patients who have received the placebo. In some embodiments, administering such treatment results in a reduction in change over baseline in Changes in Subject Memory Checklist score after treatment of about five years or longer.

In some embodiments, the genetic mutation that causes familial AD is an autosomal dominant mutation causing Autosomal Dominant Alzheimer's Disease (ADAD). In some embodiments, the ADAD comprises one or more mutations in one or more genes selected from the group consisting of presenilin 1 (PSEN1), presenilin 2 (PSEN2), and/or amyloid precursor protein (APP).

In some embodiments, the humanized monoclonal anti-amyloid beta (Aβ) antibody is delivered intravenously.

In some embodiments, the humanized monoclonal anti-amyloid beta (Aβ) antibody is administered at a) a dose of about 60 mg/kg or higher; or b) a fixed dose of 4200 mg or higher; or c) a fixed dose of about 4200 mg. In some embodiments, the humanized monoclonal anti-amyloid beta (Aβ) antibody is delivered about every four weeks (Q4W). In some embodiments, the humanized monoclonal anti-amyloid beta (Aβ) antibody is delivered about Q4W for about 5 years.

In some embodiments, the humanized monoclonal anti-amyloid beta (Aβ) antibody is delivered subcutaneously. In some embodiments, the humanized monoclonal anti-amyloid beta (Aβ) antibody is delivered at a dose of about 720 mg or higher. In some embodiments, the humanized monoclonal anti-amyloid beta (Aβ) antibody is delivered every other week (Q2W). In some embodiments, the humanized monoclonal anti-amyloid beta (Aβ) antibody is delivered about Q2W for about 5 years. In some embodiments, the humanized monoclonal anti-amyloid beta (Aβ) antibody is delivered at a dose of about 300 mg.

In some embodiments, the humanized monoclonal anti-amyloid beta (Aβ) antibody comprises a heavy chain variable region (VH) comprising the amino acid sequence of SEQ ID NO:10 and a light chain variable region (VL) comprising the amino acid sequence of SEQ ID NO:11.

In some embodiments, the humanized monoclonal anti-amyloid beta (Aβ) antibody comprises a heavy chain comprising the amino acid sequence of SEQ ID NO:5 and a light chain comprising the amino acid sequence of SEQ ID NO:9.

In some embodiments, the humanized monoclonal anti-amyloid beta (Aβ) antibody is crenezumab.

In some embodiments, administering such treatment results in a reduced Standardized Uptake Value Ratio (SUVR) of amyloid PET measurement in the plurality of human patients as compared to a reference SUVR of amyloid PET, wherein the reference SUVR of amyloid PET is a SUVR of amyloid PET of the plurality of human patients who have received the placebo. In some embodiments, administering such treatment results in a reduced Standardized Uptake Value Ratio (SUVR) of amyloid PET measurement in the plurality of human patients by at least 3% as compared to the reference SUVR of amyloid PET. In some embodiments, administering such treatment results in a reduced Standardized Uptake Value Ratio (SUVR) of amyloid PET measurement in the plurality of human patients by at least 10% relative to the reference SUVR of amyloid PET. In some embodiments, administering such treatment results in a reduced Standardized Uptake Value Ratio (SUVR) of amyloid PET measurement in the plurality of human patients by 3% to 10% relative to the reference SUVR of amyloid PET.

In some embodiments, administering such treatment results in a reduction of cerebrospinal fluid (CSF) neurofilament light (CSF NfL) in the plurality of human patients as compared to a reference CSF NfL, wherein the reference CSF NfL is from the plurality of human patients who have received the placebo. In some embodiments, administering such treatment results in a relative reduction of CSF NfL in the plurality of human patients of at least 10% relative to the reference CSF NIL. In some embodiments, administering such treatment results in a relative reduction of CSF NfL in the plurality of human patients of at least 20% relative to the reference CSF NfL. In some embodiments, administering such treatment results in a relative reduction of CSF NfL in the plurality of human patients of 10% to 20% relative to the reference CSF NIL

In some embodiments, administering such treatment to the plurality of human patients results in an effect on a plasma biomarker compared to a reference plasma biomarker, wherein the reference plasma biomarker is the plasma biomarker of a plurality of human patients who have received the placebo. In some embodiments, the plasma biomarker is measured using an immunoassay. In some embodiments, the plasma biomarker is any one of Aβ42. Aβ40, pTau181, pTau217, NfL, GFAP, YKL-40, or sTREM2. In some embodiments, plasma biomarker is the ratio of Aβ42 to Aβ40.

In some embodiments, administering such treatment results in an increase of annualized rate of change in the plasma Aβ42 biomarker in the plurality of human patients compared to a reference annualized rate of change in the plasma Aβ42 biomarker, wherein the reference plasma Aβ42 biomarker is an annualized rate of change in the plasma Aβ42 biomarker of a plurality of human patients who have received the placebo.

In some embodiments, administering such treatment results in an increase of annualized rate of change in the plasma Aβ40 biomarker in the plurality of human patients compared to a reference annualized rate of change in the plasma Aβ40 biomarker, wherein the reference plasma Aβ40 biomarker is an annualized rate of change in the plasma Aβ40 biomarker of a plurality of human patients who have received the placebo.

In some embodiments, administering such treatment results in a reduction of annualized rate of change in the plasma pTau181 biomarker in the plurality of human patients compared to a reference annualized rate of change in the plasma pTau181 biomarker, wherein the reference plasma pTau181 biomarker is an annualized rate of change in the plasma pTau181 biomarker of a plurality of human patients who have received the placebo. In some embodiments, administering such treatment results in a reduction of annualized rate of change in the plasma pTau181 biomarker in the plurality of human patients by about 6% relative to the reference plasma pTau181 biomarker.

In some embodiments, administering such treatment results in a reduction of annualized rate of change in the plasma pTau217 biomarker in the plurality of human patients compared to a reference annualized rate of change in the plasma pTau217 biomarker, wherein the reference plasma pTau217 biomarker is an annualized rate of change in the plasma pTau217 biomarker of a plurality of human patients who have received the placebo. In some embodiments, administering such treatment results in a reduction of annualized rate of change in the plasma pTau217 biomarker in the plurality of human patients by about 9% relative to the reference plasma pTau217 biomarker.

In some embodiments, administering such treatment results in a reduction of annualized rate of change in the plasma neurofilament light (NfL) biomarker in the plurality of human patients compared to a reference annualized rate of change in the plasma NfL biomarker, wherein the reference plasma NfL biomarker is an annualized rate of change in the plasma NfL biomarker of a plurality of human patients who have received the placebo. In some embodiments, administering such treatment results in a reduction of annualized rate of change in the plasma NfL biomarker in the plurality of human patients by about 10% relative to the reference plasma NfL biomarker.

In some embodiments, administering such treatment results in a reduction of annualized rate of change in the plasma GFAP biomarker in the plurality of human patients compared to a reference annualized rate of change in the plasma GFAP biomarker, wherein the reference plasma GFAP biomarker is an annualized rate of change in the plasma GFAP biomarker of a plurality of human patients who have received the placebo. In some embodiments, administering such treatment results in a reduction of annualized rate of change in the plasma GFAP biomarker in the plurality of human patients by about 17% relative to the reference plasma GFAP biomarker.

In some embodiments, administering such treatment results in a reduction of annualized rate of change in the plasma YKL-40 biomarker in the plurality of human patients compared to a reference annualized rate of change in the plasma YKL-40 biomarker, wherein the reference plasma YKL-40 biomarker is an annualized rate of change in the plasma YKL-40 biomarker of a plurality of human patients who have received the placebo. In some embodiments, administering such treatment results in a reduction of annualized rate of change in the plasma YKL-40 biomarker in the plurality of human patients by about 12% relative to the reference plasma YKL-40 biomarker.

In some embodiments, administering such treatment results in a reduction of annualized rate of change in the plasma sTREM2 biomarker in the plurality of human patients compared to a reference annualized rate of change in the plasma sTREM2 biomarker, wherein the reference plasma sTREM2 biomarker is an annualized rate of change in the plasma sTREM2 biomarker of a plurality of human patients who have received the placebo. In some embodiments, administering such treatment results in a reduction of annualized rate of change in the plasma sTREM2 biomarker in the plurality of human patients by about 23% relative to the reference plasma sTREM2 biomarker.

In another aspect, provided herein is a kit comprising a humanized monoclonal anti-amyloid beta (Aβ) antibody for treating a human patient in need thereof having a genetic mutation that causes familial Alzheimer's Disease (AD), according to the method as described herein.

In still another aspect, provided herein a humanized monoclonal anti-amyloid beta (Aβ) antibody for use for treating a human patient in need thereof having a genetic mutation that causes familial Alzheimer's Disease (AD) according to the method as described herein.

### BRIEF DESCRIPTION OF THE DRAWINGS

FIG. 1A shows an overview of the clinical trial treatment groups. FIG. 1B shows an overview of the clinical trial design and subsection timeline.

FIG. 2A shows an overview of the clinical trial treatment groups. FIG. 2B shows an overview of the clinical trial design and changes to the dosing regimen.

FIG. 3 shows treatment exposure over the course of the trial. All participants, including both carriers and non-carriers, who received at least 1 dose of study drug are included. IV refers to intravenous administration, and SC refers to subcutaneous administration.

FIG. 4 shows results of the dual primary outcomes and key secondary outcomes. Forest plots show mean reductions in annualized rates/hazards in the crenezumab carrier group compared to that in the placebo carrier group and 95% confidence intervals. * P values are from testing the difference between crenezumab carrier and placebo carrier groups and are uncorrected by multiple comparisons.

FIG. 5 shows a Kaplan-Meier curve for time to MCI or dementia due to AD. Log rank test is stratified by: baseline age, education, CDR global score, and APOE4 carrier status.

FIG. 6 shows the results of the primary outcomes by baseline amyloid status.

FIG. 7 shows baseline Aβ PET measurements. Abbreviations are as follows: Aβ, amyloid-beta; AD, Alzheimer's disease; PET, positron emission tomography; SUVR, standard uptake value ratio.

FIG. 8 shows biomarker outcomes in crenezumab and placebo-treated PSEN1 E280A mutation carrier groups. Forest plots show mean reductions in annualized rates/hazards in the crenezumab carrier group compared to that in the placebo carrier group and 95% confidence intervals. Abbreviations are as follows: Aβ, Alzheimer's disease; APOE4, apolipoprotein E4; CI, confidence interval; CSF, cerebrospinal fluid; ERC, entorhinal cortex; FDG, fluorodeoxyglucose; GTP1, Genentech Tau Probe 1; NfL, neurofilament light chain; PET, positron emission tomography; pTau, phosphorylated Tau; RCRM, random coefficient regression model; sROI, statistical region of interest; SUVR, standard uptake value ratio; tTau, total Tau; vMRI, volumetric magnetic resonance imaging. * P values are from testing the difference between crenezumab carrier and placebo carrier groups and are uncorrected by multiple comparisons.

FIG. 9 shows Amyloid-β PET changes in crenezumab and placebo-treated PSEN1 E280A mutation carrier groups. Florbetapir SUVR is the mean cortical-to-white matter florbetapir SUVR. The plot on the left shows the mixed model repeated measures (MMRM) analysis with the Y-axis showing the least squares (LS) mean change from baseline, and the plot on the right shows the Locally-Weighted Scatterplot Smoothing (LOESS). MMRM analysis was adjusted for baseline score, baseline age, education, APOE4, CDR GS, treatment, visit, baseline-by-visit interaction and treatment-by-visit interaction to estimate the mean change from baseline; P value is uncorrected for multiple comparisons.

FIG. 10 shows FDG PET changes in crenezumab and placebo-treated PSEN1 E280A mutation carrier groups. sROI FDG SUVR is a FDG (fluorodeoxyglucose) Standardized Uptake Value Ratio (SUVR) in a pre-specified statistical region of interest (ROI) that is preferentially associated with AD-related CMRg1 (cerebral metabolic rate for glucose) declines. The plot on the left shows the mixed model repeated measures (MMRM) analysis with the Y-axis showing the least squares (LS) mean change from baseline, and the plot on the right shows the Locally-Weighted Scatterplot Smoothing (LOESS). MMRM analysis was adjusted for baseline score, baseline age, education, APOE4, CDR GS, treatment, visit, baseline-by-visit interaction and treatment-by-visit interaction to estimate the mean change from baseline; P value is uncorrected for multiple comparisons.

FIG. 11 shows CSF Amyloid-B42 changes in crenezumab and placebo-treated PSEN1 E280A mutation carrier groups. The plot on the left shows the mixed model repeated measures (MMRM) analysis with the Y-axis showing the least squares (LS) mean change from baseline, and the plot on the right shows the Locally-Weighted Scatterplot Smoothing (LOESS). MMRM analysis was adjusted for baseline score, baseline age, education, APOE4, CDR GS, treatment, visit, baseline-by-visit interaction and treatment-by-visit interaction to estimate the mean change from baseline; P value is uncorrected for multiple comparisons.

FIG. 12 shows CSF Aβ40 changes in crenezumab and placebo-treated PSEN1 E280A mutation carrier groups. The plot on the left shows the mixed model repeated measures (MMRM) analysis with the Y-axis showing the least squares (LS) mean change from baseline, and the plot on the right shows the Locally-Weighted Scatterplot Smoothing (LOESS). MMRM analysis was adjusted for baseline score, baseline age, education, APOE4, CDR GS, treatment, visit, baseline-by-visit interaction and treatment-by-visit interaction to estimate the mean change from baseline; P value is uncorrected for multiple comparisons.

FIG. 13 shows CSF pTau181 changes in crenezumab and placebo-treated PSEN1 E280A mutation carrier groups. The plot on the left shows the mixed model repeated measures (MMRM) analysis with the Y-axis showing the least squares (LS) mean change from baseline, and the plot on the right shows the Locally-Weighted Scatterplot Smoothing (LOESS). MMRM analysis was adjusted for baseline score, baseline age, education, APOE4, CDR GS, treatment, visit, baseline-by-visit interaction and treatment-by-visit interaction to estimate the mean change from baseline; P value is uncorrected for multiple comparisons.

FIG. 14 shows CSF total Tau changes in crenezumab and placebo-treated PSEN1 E280A mutation carrier groups. The plot on the left shows the mixed model repeated measures (MMRM) analysis with the Y-axis showing the least squares (LS) mean change from baseline, and the plot on the right shows the Locally-Weighted Scatterplot Smoothing (LOESS). MMRM analysis was adjusted for baseline score, baseline age, education, APOE4, CDR GS, treatment, visit, baseline-by-visit interaction and treatment-by-visit interaction to estimate the mean change from baseline; P value is uncorrected for multiple comparisons.

FIG. 15 show CSF NfL (neurofilament light) changes (Log₁₀) in crenezumab and placebo-treated PSEN1 E280A mutation carrier groups. The plot on the left shows the mixed model repeated measures (MMRM) analysis with the Y-axis showing the least squares (LS) mean change from baseline, and the plot on the right shows the Locally-Weighted Scatterplot Smoothing (LOESS). MMRM analysis was adjusted for baseline score, baseline age, education, APOE4, CDR GS, treatment, visit, baseline-by-visit interaction and treatment-by-visit interaction to estimate the mean change from baseline; P value is uncorrected for multiple comparisons.

FIG. 16 shows MRI brain volume changes in crenezumab and placebo-treated PSEN1 E280A mutation carrier groups. Annualized mean percent changes reflect the mean percent differences between the first and last scan. Hippocampal and ventricular volume changes were computed using FreeSurfer (Version 7.1). Whole brain volume changes were computed using Banner's Iterative Principal Component Analysis (IPCA).

FIG. 17 shows Tau PET changes in crenezumab and placebo-treated PSEN1 E280A mutation carrier groups. Tau PET was introduced later in the study. Due to variability in tau PET visits, only the LOESS plot is shown.

FIG. 18 shows the baseline plasma biomarker findings of the study. Included participants included those who received ≥1 dose of study drug and had at ≥1 plasma measurement. The Aβ42, Aβ40 and Aβ42/40 measurements exclude two outliers in the non-carrier group. YKL-40 is in μg/mL; sTREM2 is in ng/ml, and all other Log10 concentrations are in pg/mL. Abbreviations are as follows: Aβ, amyloid-beta; GFAP, glial fibrillary acidic protein; NfL, neurofilament light chain; pTau, phosphorylated Tau; SD, standard deviation; sTREM2, soluble triggering receptor expressed on myeloid cells 2; YKL-40, chitinase-3-like protein 1.

FIG. 19 shows the association between plasma biomarkers and age. The y-axis shows the baseline plasma levels (locally estimated scatterplot smoothing); the x-axis shows participant age; each plot is labeled with the biomarker analyzed. The data excludes plasma Aβ42. Aβ40 and Aβ42/40 measurements from two outliers in the non-carrier group.

FIG. 20 shows the longitudinal plasma biomarker changes in placebo carriers and non-carriers. The y-axis shows the change from baseline (locally estimated scatterplot smoothing); the x-axis indicates the year of the study; each plot is labeled with the biomarker analyzed. The data excludes plasma Aβ42, Aβ40 and Aβ42/40 measurements from two outliers in the non-carrier group.

FIG. 21 shows plasma Aβ42 changes in crenezumab and placebo-treated carriers. The plot on the left shows the mixed model repeated measures (MMRM) analysis with the Y-axis showing the least squares (LS) mean change from baseline, and the plot on the right shows the Locally-Weighted Scatterplot Smoothing (LOESS). MMRM analysis was adjusted for baseline score, baseline age, education, APOE4, CDR GS, treatment, visit, baseline-by-visit interaction and treatment-by-visit interaction to estimate the mean change from baseline; P value is uncorrected for multiple comparisons.

FIG. 22 shows plasma Aβ40 changes in crenezumab and placebo-treated carriers. The plot on the left shows the mixed model repeated measures (MMRM) analysis with the Y-axis showing the least squares (LS) mean change from baseline, and the plot on the right shows the Locally-Weighted Scatterplot Smoothing (LOESS). MMRM analysis was adjusted for baseline score, baseline age, education, APOE4, CDR GS, treatment, visit, baseline-by-visit interaction and treatment-by-visit interaction to estimate the mean change from baseline; P value is uncorrected for multiple comparisons.

FIG. 23 shows plasma biomarker outcomes in crenezumab and placebo-treated carriers. Forest plots show mean reductions in biomarker progression in the crenezumab carrier group compared to those in the placebo carrier group and 95% CIs.

FIG. 24 shows Log10 plasma pTau181 changes in crenezumab and placebo-treated carriers. The plot on the left shows the mixed model repeated measures (MMRM) analysis with the Y-axis showing the least squares (LS) mean change from baseline, and the plot on the right shows the Locally-Weighted Scatterplot Smoothing (LOESS). MMRM analysis was adjusted for baseline score, baseline age, education, APOE4, CDR GS, treatment, visit, baseline-by-visit interaction and treatment-by-visit interaction to estimate the mean change from baseline; P value is uncorrected for multiple comparisons.

FIG. 25 shows Log10 plasma pTau217 changes in crenezumab and placebo-treated carriers. The plot on the left shows the mixed model repeated measures (MMRM) analysis with the Y-axis showing the least squares (LS) mean change from baseline, and the plot on the right shows the Locally-Weighted Scatterplot Smoothing (LOESS). MMRM analysis was adjusted for baseline score, baseline age, education, APOE4, CDR GS, treatment, visit, baseline-by-visit interaction and treatment-by-visit interaction to estimate the mean change from baseline; P value is uncorrected for multiple comparisons.

FIG. 26 shows Log10 plasma NfL changes in crenezumab and placebo-treated carriers. The plot on the left shows the mixed model repeated measures (MMRM) analysis with the Y-axis showing the least squares (LS) mean change from baseline, and the plot on the right shows the Locally-Weighted Scatterplot Smoothing (LOESS). MMRM analysis was adjusted for baseline score, baseline age, education, APOE4, CDR GS, treatment, visit, baseline-by-visit interaction and treatment-by-visit interaction to estimate the mean change from baseline; P value is uncorrected for multiple comparisons.

FIG. 27 shows Log10 plasma GFAP changes in crenezumab and placebo-treated carriers. The plot on the left shows the mixed model repeated measures (MMRM) analysis with the Y-axis showing the least squares (LS) mean change from baseline, and the plot on the right shows the Locally-Weighted Scatterplot Smoothing (LOESS). MMRM analysis was adjusted for baseline score, baseline age, education, APOE4, CDR GS, treatment, visit, baseline-by-visit interaction and treatment-by-visit interaction to estimate the mean change from baseline; P value is uncorrected for multiple comparisons.

FIG. 28 shows Log10 plasma YKL-40 changes in crenezumab and placebo-treated carriers. The plot on the left shows the mixed model repeated measures (MMRM) analysis with the Y-axis showing the least squares (LS) mean change from baseline, and the plot on the right shows the Locally-Weighted Scatterplot Smoothing (LOESS). MMRM analysis was adjusted for baseline score, baseline age, education, APOE4, CDR GS, treatment, visit, baseline-by-visit interaction and treatment-by-visit interaction to estimate the mean change from baseline; P value is uncorrected for multiple comparisons.

FIG. 29 shows Log10 plasma sTREM2 changes in crenezumab and placebo-treated carriers. The plot on the left shows the mixed model repeated measures (MMRM) analysis with the Y-axis showing the least squares (LS) mean change from baseline, and the plot on the right shows the Locally-Weighted Scatterplot Smoothing (LOESS). MMRM analysis was adjusted for baseline score, baseline age, education, APOE4, CDR GS, treatment, visit, baseline-by-visit interaction and treatment-by-visit interaction to estimate the mean change from baseline; P value is uncorrected for multiple comparisons.

### DETAILED DESCRIPTION

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

All references referred to in this application are hereby incorporated by reference in their entireties.

### I. Definitions

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

As used in this specification and the appended claims, the singular forms “a,” “an” and “the” include plural referents unless the context clearly dictates otherwise. Thus, for example, reference to “a protein” or an “antibody” includes a plurality of proteins or antibodies, respectively; reference to “a cell” includes mixtures of cells, and the like.

Ranges provided in the specification and appended claims include both endpoints and all points between the endpoints. Thus, for example, a range of 2.0 to 3.0 includes 2.0, 3.0, and all points between 2.0 and 3.0.

The term “sample,” or “test sample” as used herein, refers to a composition that is obtained or derived from a subject of interest that contains a cellular and/or other molecular entity that is to be characterized and/or identified, for example based on physical, biochemical, chemical and/or physiological characteristics. In one embodiment, the definition encompasses blood and other liquid samples of biological origin and tissue samples such as a biopsy specimen or tissue cultures or cells derived therefrom. The source of the tissue sample may be solid tissue as from a fresh, frozen and/or preserved organ or tissue sample or biopsy or aspirate; blood or any blood constituents; bodily fluids, including cerebrospinal fluid; and cells from any time in gestation or development of the subject or plasma. The term “biological sample” as used herein includes, but is not limited to, blood, serum, plasma, sputum, and tissue biopsies (e.g., brain samples).

The term “sample.” “biological sample,” or “test sample” includes biological samples that have been manipulated in any way after their procurement, such as by treatment with reagents, solubilization, or enrichment for certain components, such as proteins or polynucleotides, or embedding in a semi-solid or solid matrix for sectioning purposes. For the purposes herein a “section” of a tissue sample is meant a single part or piece of a tissue sample, e.g. a thin slice of tissue or cells cut from a tissue sample. Samples include, but are not limited to, whole blood, blood-derived cells, serum, plasma, lymph fluid, synovial fluid, cellular extracts, and combinations thereof. In one embodiment, the sample is a clinical sample. In another embodiment, the sample is used in a diagnostic assay.

In one embodiment, a sample is obtained from a subject or patient prior to treatment with an anti-Aβ antibody. In another embodiment, a sample is obtained from a subject or patient following at least one treatment with an anti-Aβ antibody.

A “reference” or a “reference sample,” as used herein, refers to any sample, standard, or level that is used for comparison purposes. In some embodiments, the reference is a measurement obtained from an individual who has not received treatment with an anti-Aβ antibody. In some embodiments, the reference is a measurement obtained from an individual receiving a placebo.

In certain embodiments, a reference sample is a single sample or combined multiple samples from the same subject or patient that are obtained at one or more different time points than when the test sample is obtained. For example, a reference sample is obtained at an earlier time point from the same subject or patient than when the test sample is obtained. In certain embodiments, a reference sample includes all types of biological samples as defined above under the term “sample” that is obtained from one or more individuals who is not the subject or patient. In some embodiments, a reference sample is obtained from a subject who received a placebo. In certain embodiments, a reference sample is obtained from one or more individuals with Alzheimer's Disease, who is not the subject or patient.

In certain embodiments, a reference sample is a combined multiple samples from one or more healthy individuals who are not the subject or patient. In certain embodiments, a reference sample is a combined multiple samples from one or more individuals with a disease or disorder (e.g., Alzheimer's Disease) who are not the subject or patient. In certain embodiments, a reference sample is pooled RNA samples from normal tissues or pooled plasma or serum samples from one or more individuals who are not the subject or patient.

The term “small molecule” refers to an organic molecule having a molecular weight between 50 Daltons to 2500 Daltons.

The terms “antibody” and “immunoglobulin” (“Ig”) are used interchangeably in the broadest sense and include, but are not limited to, monoclonal antibodies (for example, full length or intact monoclonal antibodies), polyclonal antibodies, multivalent antibodies, antibodies with polyepitopic specificity, single chain antibodies, multi-specific antibodies (for example, bispecific antibodies, trispecific antibodies, tetraspecific antibodies), and fragments of antibodies, provided they exhibit the desired biological activity. Such antibodies can be chimeric, humanized, human, synthetic, and/or affinity matured. Such antibodies and methods of generating them are described in more detail herein.

“Antibody fragments” comprise only a portion of an intact antibody, wherein the portion preferably retains at least one, and typically most or all, of the functions normally associated with that portion when present in an intact antibody. In one embodiment, an antibody fragment comprises an antigen binding site of the intact antibody and thus retains the ability to bind antigen. In another embodiment, an antibody fragment, for example one that comprises the Fc region, retains at least one of the biological functions normally associated with the Fc region when present in an intact antibody, such as FcRn binding, antibody half-life modulation, ADCC function and complement binding. In one embodiment, an antibody fragment is a monovalent antibody that has an in vivo half-life substantially similar to an intact antibody. For example, such an antibody fragment may comprise an antigen binding arm linked to an Fe sequence capable of conferring in vivo stability to the fragment. Examples of antibody fragments include but are not limited to Fv. Fab, Fab′, Fab′-SH, F(ab′)2; diabodies; linear antibodies; single-chain antibody molecules (e.g. scFv); and multispecific antibodies formed from antibody fragments.

The term “target.” as used herein, refers to any native molecule from any vertebrate source, including mammals such as primates (e.g. humans) and rodents (e.g., mice and rats), unless otherwise indicated. The term encompasses “full-length,” unprocessed target as well as any form of target that results from processing in the cell. The term also encompasses naturally occurring variants of targets, e.g., splice variants or allelic variants.

The terms “amyloid beta,” “beta-amyloid,” “Abeta,” “amyloidβ.” and “Aβ”, used interchangeably herein, refer to the fragment of amyloid precursor protein (“APP”) that is produced upon β-secretase 1 (“BACE1”) cleavage of APP, as well as modifications, fragments and any functional equivalents thereof, including, but not limited to, Aβ1-40, and Aβ1-42. Aβ is known to exist in monomeric form, as well as to associate to form oligomers and fibril structures, which may be found as constituent members of amyloid plaque. The structure and sequences of such Aβ peptides are well known to be an ordinary skill in the art and methods of producing said peptides or of extracting them from brain and other tissues are described, for example, in Glenner and Wong, Biochem Biophys Res. Comm. 129:885-890 (1984). Moreover, Aβ peptides are also commercially available in various forms. An exemplary amino acid sequence of human Aβ1-42 is DAEFRHDSGYEVHHQKLVFFAED VGSNKGAIIGLMVGGVVIA (SEQ ID NO: 1). “Anti-Aβ immunoglobulin,” “anti-Aβ antibody,” and “antibody that binds Aβ” are used interchangeably herein, and refer to an antibody that specifically binds to human Aβ. A non-limiting example of an anti-Aβ antibody is crenezumab.

The terms “crenezumab” and “MABT5102A” are used interchangeably herein, and refer to a specific anti-Aβ antibody that binds to monomeric, oligomeric, and fibril forms of Aβ, and which is associated with CAS registry number 1095207. Crenezumab comprises: (1) an HVR-H1 comprising the amino acid sequence SEQ ID NO: 2; (2) an HVR-H2 sequence comprising the amino acid sequence SEQ ID NO: 3; (3) an HVR-H3 sequence comprising the amino acid sequence SEQ ID NO: 4; (4) an HVR-L1 sequence comprising the amino acid sequence SEQ ID NO: 6; (5) an HVR-L2 sequence comprising the amino acid sequence SEQ ID NO: 7; and (6) an HVR-L3 sequence comprising the amino acid sequence SEQ ID NO: 8. In some embodiments, the specific anti-Aβ antibody comprises a heavy chain comprising the amino acid sequence SEQ ID NO: 5 and a light chain comprising the amino acid sequence SEQ ID NO: 9. In another such embodiment, such specific anti-Aβ antibody comprises a VH domain comprising the amino acid sequence SEQ ID NO: 10 and a VL domain comprising the amino acid sequence SEQ ID NO: 11. In another embodiment, the antibody is an IgG4 antibody. In another such embodiment, the IgG4 antibody comprises a mutation in its constant domain such that serine 228 is instead a proline.

The term “familial AD” refers to Alzheimer's Disease wherein the patient has a family history of AD and/or is a carrier of a genetic mutation that causes AD and/or impacts the progression of AD.

The term “autosomal dominant AD” or “ADAD” refers to AD wherein the genetic mutation is an autosomal dominant mutation, such that being a carrier for the mutation is associated with a high likelihood of developing AD and/or AD progression in the carrier.

The term “therapeutic agent” refers to any agent that is used to treat a disease, including but not limited to an agent that treats a symptom of the disease.

As used herein, “treatment” (and grammatical variations thereof such as “treat” or “treating”) refers to clinical intervention in an attempt to alter the natural course of the individual being treated, and can be performed during the course of clinical pathology. Desirable effects of treatment include, but are not limited to, alleviation or amelioration of one or more symptoms, diminishment of or delay in the appearance of or worsening of any direct or indirect pathological consequences of the disease, decrease of the rate of disease progression, and amelioration or palliation of the disease state. In some embodiments, antibodies are used to delay development of a disease or to slow the progression of a disease such as AD.

The term “treatment emergent” as used herein refers to an event that occurs after a first dose of a therapeutic agent is administered. For example, a “treatment emergent adverse event” is an event that is identified upon or after the first dose of a treatment in a clinical study.

“Treatment regimen” refers to a combination of dosage, frequency of administration, or duration of treatment, with or without addition of a second medication.

“Effective treatment regimen” refers to a treatment regimen that will offer beneficial response to a patient receiving the treatment.

“Modifying a treatment” refers to changing the treatment regimen including, changing dosage, frequency of administration, or duration of treatment, and/or addition of a second medication.

An “effective amount” or “effective dose” of an agent refers to an amount or dose effective, for periods of time necessary, to achieve the desired result. For example, a “therapeutically effective amount” is an amount effective, for periods of time necessary, to treat the indicated disease, condition, clinical pathology, or symptom, i.e., to modify the course of progression of AD and/or to alleviate and/or prevent one or more symptoms of AD.

“Affinity” or “binding affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen binding arm). The affinity of a molecule X for its partner Y can generally be represented by the dissociation constant (Kd). Affinity can be measured by common methods known in the art, including those described herein, any of which can be used for purposes of the present invention. Specific illustrative and exemplary embodiments for measuring binding affinity are described herein.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

As used herein, the term “patient” refers to any single subject for which treatment is desired. In certain embodiments, the patient herein is a human.

A “subject” herein is typically a human. Typically, the subject is eligible for treatment, e.g., has a genetic mutation associated with AD, such as a mutation that causes or is predicted to cause familial AD. An eligible subject or patient is at risk for developing AD based on a genetic mutation. A subject may not have any signs or symptoms of AD such as MCI. Diagnosis of AD or a risk of developing AD may be made based on clinical history, clinical examination, established imaging modalities, and/or genetic testing. A “patient” or “subject” herein includes any single human subject eligible for treatment who has a genetic mutation that causes familial AD. Intended to be included as a subject are any subjects involved in clinical research trials, or subjects involved in epidemiological studies, or subjects once used as controls.

As used herein, “lifetime” of a subject refers to the remainder of the life of the subject after starting treatment.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 1984; 81:6851-6855).

The “class” of an antibody refers to the type of constant domain or constant region possessed by its heavy chain. There are five major classes of antibodies: IgA, IgD, IgE, IgG, and IgM, and several of these may be further divided into subclasses (or “isotypes”). e.g., IgG1, IgG2, IgG3, IgG4, IgA1, and IgA2. The heavy chain constant domains that correspond to the different classes of immunoglobulins are called α, δ, ε, γ, and μ. respectively.

“Humanized” forms of non-human (e.g., murine) antibodies are chimeric antibodies that contain minimal sequences derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin lo sequence. The humanized antibody optionally will also comprise at least a portion of an immunoglobulin constant region (Fc), typically that of a human immunoglobulin. For further details, see Jones et al., Nature, 1986; 321:522-525; Riechmann et al., Nature, 1988; 332:323-329; and Presta, Curr. Op. Struct. Biol . . . 1992; 2:593-596. See also the following review articles and references cited therein: Vaswani and Hamilton, Ann. Allergy, Asthma & Immunol., 1998; 1:105-115; Harris, Biochem. Soc. Transactions, 1995; 23:1035-1038; Hurle and Gross, Curr. Op. Biotech., 1994; 5:428-433.

A “human antibody” is one which comprises an amino acid sequence corresponding to that of an antibody produced by a human or a human cell and/or has been derived from a non-human source that utilizes human antibody repertoires or other human antibody-encoding sequences, for example made using any of the techniques for making human antibodies as disclosed herein. Such techniques include, but are not limited to, screening human-derived combinatorial libraries, such as phage display libraries (see, e.g., Marks et al., J. Mol. Biol., 1991; 222:581-597 and Hoogenboom et al., Nucl. Acids Res., 1991; 19:4133-4137); using human myeloma and mouse-human heteromyeloma cell lines for the production of human monoclonal antibodies (see, e.g., Kozbor J. Immunol., 1984; 133:3001; Brodeur et al., Monoclonal Antibody Production Techniques and Applications, pp. 55-93 (Marcel Dekker, Inc., New York, 1987); and Boerner et al., J. Immunol., 1991; 147:86); and generating monoclonal antibodies in transgenic animals (e.g., mice) that are capable of producing a full repertoire of human antibodies in the absence of endogenous immunoglobulin production (see, e.g., Jakobovits et al., Proc. Natl. Acad. Sci USA, 1993; 90:2551; Jakobovits et al., Nature, 1993; 362:255; Bruggermann et al., Year in Immunol., 1993; 7:33). This definition of a human antibody specifically excludes a humanized antibody comprising antigen-binding residues from a non-human animal.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaccous or nonproteinaceous solutes. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B. 2007; 848:79-87.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (Sec, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol., 1993; 150:880-887; Clarkson et al., Nature, 1991; 352:624-628.

The term “hypervariable region,” “HVR,” or “HV,” when used herein refers to the regions of an antibody variable domain which are hypervariable in sequence and/or form structurally defined loops. Generally, antibodies comprise six hypervariable regions; three in the VH (H1, H2, H3), and three in the VL (L1, L2, L3). A number of hypervariable region delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol., 1987; 196:901-917). The AbM hypervariable regions represent a compromise between the Kabat CDRs and Chothia structural loops, and are used by Oxford Molecular's AbM antibody modeling software. The “contact” hypervariable regions are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat Numbering) |
| H1 | H31-H35 | H26-H35 | H26-H32 | H30-H35 |
| | | | | (Chothia Numbering) |
| H2 | H50-H65 | H50-H58 | H53-H55 | H47-H58 |
| H3 | H95-H102 | H95-H102 | H96-H101 | H93-H101 |

Hypervariable regions may comprise “extended hypervariable regions” as follows: 24-36 or 24-34 (L1), 46-56 or 49-56 or 50-56 or 52-56 (L2) and 89-97 (L3) in the VL and 26-35 (H1), 50-65 or 49-65 (H2) and 93-102, 94-102 or 95-102 (H3) in the VH. The variable domain residues are numbered according to Kabat et al., supra for each of these definitions.

“Framework” or “FR” residues are those variable domain residues other than the hypervariable region residues as defined herein. The FR of a variable domain generally consists of four FR domains: FR1, FR2, FR3, and FR4. Accordingly, the HVR and FR sequences generally appear in the following sequence in VH (or VL): FR1-H1 (L1)-FR2-H2 (L2)-FR3-H3 (L3)-FR4.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

A “human consensus framework” is a framework which represents the most commonly occurring amino acid residue in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al. Sequences of Proteins of Immunological Interest, Fifth Edition, NIH Publication 91-3242, Bethesda M D (1991), vols. 1-3.

The term “Amyloid-Related Imaging Abnormality-Edema” or “ARIA-E” encompasses cerebral vasogenic edema and sulcal effusion.

The term “Amyloid-Related Imaging Abnormality-Hemorrhage” or “ARIA-H” encompasses microhemorrhage and superficial siderosis of the central nervous system.

“Apolipoprotein E4 carrier” or “ApoE4 carrier,” used interchangeably herein with “apolipoprotein E4 positive” or “ApoE4 positive.” refers to an individual having at least one apolipoprotein E4 (or “ApoE4”) allele. An individual with zero ApoE4 alleles is referred to herein as being “ApoE4 negative” or an “ApoE4 non-carrier.” See also Prekumar, et al., Am. J Pathol., 1996; 148:2083-95.

The term “cerebral vasogenic edema” refers to an excess accumulation of intravascular fluid or protein in the intracellular or extracellular spaces of the brain. Cerebral vasogenic edema is detectable by, e.g., brain MRI, including, but not limited to FLAIR MRI, and can be asymptomatic (“asymptomatic vasogenic edema”) or associated with neurological symptoms, such as confusion, dizziness, vomiting, and lethargy (“symptomatic vasogenic edema”) (see Sperling et al. Alzheimer's & Dementia, 2001; 7:367).

The term “cerebral macrohemorrhage” refers to an intracranial hemorrhage, or bleeding in the brain, of an area that is more than about 1 cm in diameter. Cerebral macrohemorrhage is detectable by, e.g., brain MRI, including but not limited to T2*-weighted GRE MRI, and can be asymptomatic (“asymptomatic macrohemorrhage”) or associated with symptoms such as transient or permanent focal motor or sensory impairment, ataxia, aphasia, and dysarthria (“symptomatic macrohemorrhage”) (see, e.g., Chalela J A, Gomes J. Expert Rev. Neurother. 2004 4:267, 2004 and Sperling et al. Alzheimer's & Dementia, 2011; 7:367).

The term “cerebral microhemorrhage” refers to an intracranial hemorrhage, or bleeding in the brain, of an area that is less than about 1 cm in diameter. Cerebral microhemorrhage is detectable by, e.g., brain MRI, including, but not limited to T2*-weighted GRE MRI, and can be asymptomatic (“asymptomatic microhemorrhage”) or can potentially be associated with symptoms such as transient or permanent focal motor or sensory impairment, ataxia, aphasia, and dysarthria (“symptomatic microhemorrhage”). See, e.g., Greenberg, et al., Lancet Neurol., 2009; 8:165-74.

The term “sulcal effusion” refers to an effusion of fluid in the furrows, or sulci, of the brain. Sulcal effusions are detectable by, e.g., brain MRI, including but not limited to FLAIR MRI. See Sperling et al. Alzheimer's & Dementia, 2011; 7:367.

The term “superficial siderosis of the central nervous system” refers to bleeding or hemorrhage into the subarachnoid space of the brain and is detectable by, e.g., brain MRI, including but not limited to T2*-weighted GRE MRI. Symptoms indicative of superficial siderosis of the central nervous system include sensorineural deafness, cerebellar ataxia, and pyramidal signs. See Kumara-N, Am J Neuroradiol., 2010; 31:5.

The term “progression” as used herein refers to the worsening or advancement of a disease or condition over time. For example, progression may refer to a patient or subject as having mild cognitive impairment or dementia who previously did not exhibit symptoms characteristic of MCI or was not classified as having MCI. The “progression rate” or “rate of progression” of a disease refers to how fast or slow a disease develops over time in a patient diagnosed with the disease. The progression rate of a disease can be represented by measurable changes over time of particular characteristics of the disease. A patient carrying particular genetic trait is said to have, or more likely to have, “increased progression rate” if her disease state progresses faster than those patients without such genetic trait. On the other hand, a patient responding to a therapy is said to have, or more likely to have, “decreased progression rate” if her disease progression slows down after the therapy, when compared to her disease state prior to the treatment or to other patients without the treatment.

“More likely to respond” as used herein refers to patients that are most likely to demonstrate a slowing down or prevention of progression of AD. With regard to AD, “more likely to respond” refers to patients that are most likely to demonstrate a reduction in loss of function or cognition with treatment. The phrase “responsive to” in the context of the present invention indicates that a patient suffering from, being suspected to suffer or being prone to suffer from, or diagnosed with a disorder as described herein, shows a response to anti-Aβ treatment.

As used herein, the term “benefit or benefits” (e.g., benefit of crenezumab treatment) refers to a favorable change in clinical parameter relative to or compared to a baseline (i.e. prior to treatment or without treatment) or to placebo treatment. Thus, a benefit or benefits of crenezumab treatment may include, but not limited, delay onset of at least one symptom of Alzheimer's Disease (AD), slowing cognitive decline, or prevention of cognitive impairment in AD patients or individuals who show no or early symptoms of AD but are deemed at risk of AD relative to or compared to a baseline (i.e. prior to treatment or without treatment) or placebo treatment.

The phrase “selecting a patient” or “identifying a patient” as used herein refers to using the information or data generated relating to the presence of an allele in a sample of a patient to identify or select the patient as more likely to benefit from a treatment comprising an anti-Aβ antibody. The information or data used or generated may be in any form, written, oral or electronic. In some embodiments, using the information or data generated includes communicating, presenting, reporting, storing, sending, transferring, supplying, transmitting, dispensing, or combinations thereof. In some embodiments, communicating, presenting, reporting, storing, sending, transferring, supplying, transmitting, dispensing, or combinations thereof are performed by a computing device, analyzer unit or combination thereof. In some further embodiments, communicating, presenting, reporting, storing, sending, transferring, supplying, transmitting, dispensing, or combinations thereof are performed by a laboratory or medical professional. In some embodiments, the information or data includes an indication that a specific allele is present or absent in the sample. In some embodiments, the information or data includes an indication that the patient is more likely to respond to a therapy comprising anti-Aβ.

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation. It is known in the art that wild-type IgG4 antibodies have less effector function than wild-type IgG1 antibodies.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD (1991).

The terms “full length antibody.” “intact antibody.” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

An “immunoconjugate” is an antibody conjugated to one or more heterologous molecule(s), including but not limited to a further therapeutic agent.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-Aβ antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “early Alzheimer's Disease” or “early AD” as used herein (e.g., a “patient diagnosed with early AD” or a “patient having early AD”) includes patients with mild cognitive impairment, such as a memory deficit, due to AD and patients having AD biomarkers, for example amyloid positive patients, as well as patients with prodromal AD and mild AD. In some embodiments, patients with early AD have an MMSE of 22 or greater and a CDR global score of 0.5 or 1.0.

The term “cognitive impairment” as used herein refers to the difficulty of forming new memories, learning new tasks or information, concentrating, or making everyday decisions. Cognitive impairment can range from mild to severe impairments.

The term “cognitive decline” as used herein refers to the increase in cognitive impairment over time, often but not always due to aging. Cognitive decline may occur at various paces, including gradually, rapidly, and/or intermittently.

The term “delay” as used herein refers to a reduction in a specific degree of severity or an increase in the length of time to reach a specific degree of severity, e.g. of AD symptoms.

The term “onset” as used herein refers to the first instance or the beginning of a pattern of, e.g. behaviors or symptoms such as may commonly be used to diagnose a patient with AD.

The term “symptom” as used herein refers to the physical or cognitive manifestation of a disease, e.g. AD.

The term “delay onset of at least one symptom” as used herein refers to the reduction in severity of one or more symptoms, e.g. of AD, or to an increase in the length of time before initial onset of one or more symptoms, e.g. of AD.

A “naked antibody” refers to an antibody that is not conjugated to a heterologous moiety (e.g., a further therapeutic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (2), based on the amino acid sequence of its constant domain.

The term “package insert” is used to refer to instructions customarily included in commercial packages of therapeutic products, that contain information about the indications, usage, dosage, administration, combination therapy, contraindications and/or warnings concerning the use of such therapeutic products. The term “package insert” is also used to refer to instructions customarily included in commercial packages of diagnostic products that contain information about the intended use, test principle, preparation and handling of reagents, specimen collection and preparation, calibration of the assay and the assay procedure, performance and precision data such as sensitivity and specificity of the assay.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction (X/Y); where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “pharmaceutical formulation” and “pharmaceutical composition” are used interchangeably herein and refer to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

An “imaging agent” is a compound that has one or more properties that permit its location to be detected directly or indirectly. Examples of such imaging agents include proteins and small molecule compounds incorporating a labeled moiety that permits detection.

A “label” is a marker coupled with a molecule to be used for detection or imaging. Examples of such labels include: a radiolabel, a fluorophore, a chromophore, or an affinity tag. In one embodiment, the label is a radiolabel used for medical imaging, for example tritium, Technetium-99m or I-123, or a spin label for nuclear magnetic resonance (NMR) imaging (also known as magnetic resonance imaging, MRI), such as iodine-123 iodine-131, indium-111, fluorine-19, carbon-13, nitrogen-15, oxygen-17, gadolinium-DTPA, manganese-51, manganese-52g, iron oxide, etc.

The term “prevent or prevention,” as used herein when used in connection with the occurrence of a disease, disorder, and/or condition, refers to reducing the risk of developing the disease, disorder and/or condition and/or to delaying onset or progression of one or more characteristics or symptoms of the disease, disorder or condition.

### II. Methods

The present disclosure provides methods for the treatment of Autosomal Dominant Alzheimer's Disease (ADAD).

In one aspect, provided herein are methods of delaying onset of at least one symptom in a human patient with a genetic mutation that causes familial AD comprising administering to the human patient an effective amount of an anti-Aβ antibody (e.g., crenezumab), wherein administering such treatment to a plurality of human patients results in a delayed onset of at least one symptom in the plurality of human patients relative to a reference onset of at least one symptom, wherein the reference onset of at least one symptom is of a plurality of human patients who have received a placebo. In some embodiments, the antibody comprises the HVR-H1 amino acid sequence of SEQ ID NO:2, the HVR-H2 amino acid sequence of SEQ ID NO: 3, and the HVR-H3 amino acid sequence of SEQ ID NO:4; and the HVR-L1 amino acid sequence of SEQ ID NO: 6, the HVR-L2 amino acid sequence of SEQ ID NO: 7, and the HVR-L3 amino acid sequence of SEQ ID NO: 8.

In another aspect, provided herein are methods of slowing cognitive decline in a human patient with a genetic mutation that causes familial AD comprising administering to the human patient an effective amount of an anti-Aβ antibody (e.g., crenczumab), wherein administering such treatment to a plurality of human patients results in delayed cognitive decline in the plurality of human patients relative to a reference cognitive decline, wherein the reference cognitive decline is of a plurality of human patients who have received a placebo. In some embodiments, the antibody comprises the HVR-H1 amino acid sequence of SEQ ID NO:2, the HVR-H2 amino acid sequence of SEQ ID NO: 3, and the HVR-H3 amino acid sequence of SEQ ID NO:4; and the HVR-L1 amino acid sequence of SEQ ID NO: 6, the HVR-L2 amino acid sequence of SEQ ID NO: 7, and the HVR-L3 amino acid sequence of SEQ ID NO: 8.

In another aspect, provided herein are methods of preventing cognitive impairment in a human patient with a genetic mutation that causes familial AD comprising administering to the human patient an effective amount of an anti-Aβ antibody (e.g., crenezumab), wherein administering such treatment to a plurality of human patients results in reduced cognitive impairment in the plurality of human patients relative to a reference cognitive impairment, wherein the reference cognitive impairment is of a plurality of human patients who have received a placebo. In some embodiments, the antibody comprises the HVR-H1 amino acid sequence of SEQ ID NO:2, the HVR-H2 amino acid sequence of SEQ ID NO: 3, and the HVR-H3 amino acid sequence of SEQ ID NO:4; and the HVR-L1 amino acid sequence of SEQ ID NO: 6, the HVR-L2 amino acid sequence of SEQ ID NO: 7, and the HVR-L3 amino acid sequence of SEQ ID NO: 8.

### A. Antibodies

In one aspect, the methods of the present invention comprise administering antibodies that bind to Aβ. In some embodiments, the method comprises an anti-Aβ antibody that can bind to monomeric, oligomeric and fibril forms of human Aβ with good affinity. In some embodiments, the anti-Aβ antibody is an antibody that binds to an epitope of Aβ within residues 13-24 of Aβ. In some embodiments, the anti-Aβ antibody specifically binds to residues 13-24 of Aβ in an extending conformation. While not intending to be bound by any theory of operation, binding Aβ in an extended conformation is thought to account for the ability of exemplary antibodies to bind to different forms of human Aβ, including monomeric, oligomeric, and fibrillary forms. See Ultsch et al., 2016. In some embodiments, the antibody is crenezumab. In some embodiments, crenezumab binds both Aβ1-40 and Aβ1-42. In some embodiments, crenezumab inhibits Aβ aggregation. In some embodiments, crenezumab promotes Aβ disaggregation.

In one embodiment, the antibody comprises the heavy chain amino acid sequence set forth in SEQ ID NO:5 and the light chain amino acid sequence set forth in SEQ ID NO: 9. In another embodiment, the antibody comprises the heavy chain variable region of amino acids 1 to 112 of the amino acid sequence set forth in SEQ ID NO:5 and the light chain variable region of amino acids 1 to 112 of the amino acid sequence set forth in SEQ ID NO: 9. In some embodiments, the antibody comprises the heavy chain variable region sequence set forth in SEQ ID NO:10 and the light chain variable region sequence set forth in SEQ ID NO:11. In another embodiment, the antibody comprises the HVR-H1 amino acid sequence of SEQ ID NO:2, the HVR-H2 amino acid sequence of SEQ ID NO: 3, and the HVR-H3 amino acid sequence of SEQ ID NO:4; and the HVR-L1 amino acid sequence of SEQ ID NO: 6, the HVR-L2 amino acid sequence of SEQ ID NO: 7, and the HVR-L3 amino acid sequence of SEQ ID NO: 8. In another embodiment, the antibody comprises a heavy chain variable region comprising sequences that are 95%, 96%, 97%, 98%, or 99% or more identical to the amino acid sequence of SEQ ID NO:5. In another embodiment, the antibody comprises a light chain variable region comprising sequences that are 95%, 96%, 97%, 98%, or 99% or more identical to the amino acid sequence of SEQ ID NO:9.

In any of the above embodiments, an anti-Aβ antibody is humanized. In one embodiment, an anti-Aβ antibody comprises HVRs as in any of the above embodiments, and further comprises an acceptor human framework, e.g. a human immunoglobulin framework or a human consensus framework.

In another aspect, an anti-Aβ antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:5. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-Aβ antibody comprising that sequence retains the ability to bind to Aβ. In certain embodiments, a total of 1 to 10 amino acids have been substituted, inserted and/or deleted in SEQ ID NO:10. In certain embodiments, substitutions, insertions, or deletions occur in regions outside the HVRs (i.e., in the FRs). Optionally, the anti-Aβ antibody comprises the VH sequence in SEQ ID NO:10, including post-translational modifications of that sequence. In another aspect, an anti-Aβ antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:11. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-Aβ antibody comprising that sequence retains the ability to bind to Aβ. In certain embodiments, a total of 1 to 10 amino acids have been substituted, inserted and/or deleted in SEQ ID NO:11. In certain embodiments, the substitutions, insertions, or deletions occur in regions outside the HVRs (i.e., in the FRs). Optionally, the anti-Aβ antibody comprises the VL sequence in SEQ ID NO:11, including post-translational modifications of that sequence.

In another aspect, an anti-Aβ antibody is provided, wherein the antibody comprises a VH as in any of the embodiments provided above, and a VL as in any of the embodiments provided above.

In a further aspect of the invention, an anti-Aβ antibody according to any of the above embodiments is a monoclonal antibody, including a chimeric, humanized or human antibody. In one embodiment, an anti-Aβ antibody is an antibody fragment, e.g., a Fv, Fab, Fab′, scFv, diabody, or F (ab′) 2 fragment. In another embodiment, the antibody is a full length antibody, e.g., an intact IgG4 antibody or other antibody class or isotype as defined herein. In another embodiment, the antibody is a bispecific antibody.

In one embodiment, the anti-Aβ antibody comprises a HVR-L1 comprising amino acid sequence SEQ ID NO:6; an HVR-L2 comprising amino acid sequence SEQ ID NO:7; an HVR-L3 comprising amino acid sequence SEQ ID NO: 8; an HVR-H1 comprising amino acid sequence SEQ ID NO:2; an HVR-H2 comprising amino acid sequence SEQ ID NO: 3; and an HVR-H3 comprising amino acid sequence SEQ ID NO: 4. In some embodiments, the antibody is humanized. In some embodiments, the antibody is an IgG4 isotype.

In some embodiments, the antibody comprises a heavy chain variable region comprising the amino acid sequence set forth in SEQ ID NO: 10 and a light chain variable region comprising the amino acid sequence set forth in SEQ ID NO:11. In some embodiments, the antibody comprises a heavy chain comprising the amino acid sequence set forth in SEQ ID NO:5 and a light chain comprising the amino acid sequence set forth in SEQ ID NO: 9.

In any of the above embodiments, an anti-Aβ antibody can be humanized. In one embodiment, an anti-Aβ antibody comprises HVRs as in any of the above embodiments, and further comprises an acceptor human framework, e.g. a human immunoglobulin framework or a human consensus framework.

In certain embodiments, the anti-Aβ antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs. (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature, 1988; 332:323-329; Queen et al., Proc. Nat'l Acad. Sci. USA, 1989; 86:10029-10033; U.S. Pat. Nos. 5,821,337, 7,527,791, 6,982,321, and 7,087,409; Kashmiri et al., Methods, 2005; 36:25-34 (describing SDR (a-CDR) grafting); Padlan, Mol. Immunol., 1991; 28:489-498 (describing “resurfacing”); Dall'Acqua et al., Methods, 2005; 36:43-60 (describing “FR shuffling”); and Osbourn et al., Methods, 2005; 36:61-68 and Klimka et al., Br. J. Cancer, 2000; 83:252-260 (describing the “guided selection” approach to FR shuffling).

Human framework regions that may be used for humanization include but are not limited to: framework regions selected using the “best-fit” method (see, e.g., Sims et al. J. Immunol., 1993; 151:2296); framework regions derived from the consensus sequence of human antibodies of a particular subgroup of light or heavy chain variable regions (see, e.g., Carter et al. Proc. Natl. Acad. Sci. USA, 1992; 89:4285; and Presta et al. J. Immunol., 1993; 151:2623); human mature (somatically mutated) framework regions or human germline framework regions (see, e.g., Almagro and Fransson, Front. Biosci., 2008; 13:1619-1633); and framework regions derived from screening FR libraries (see, e.g., Baca et al., J. Biol. Chem., 1997; 272:10678-10684 and Rosok et al., J. Biol. Chem., 1996; 271:22611-22618).

### B. Dosing and Administration

The anti-amyloid beta (Aβ) antibodies for use in the methods described herein are formulated, dosed, and administered in a fashion consistent with good medical practice. Factors for consideration include the particular subject being treated (e.g. gender, age, weight, etc.), the clinical condition of the subject (e.g., the severity of the disease), the particular form of autosomal dominant Alzheimer's disease, the site of delivery of the antibody, the method of administration, the scheduling of administration, and other factors known to medical practitioners.

### Pharmaceutical Formulations

Pharmaceutical formulations of the anti-Aβ antibodies for use in the methods described herein are prepared by mixing such antibody or molecule having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

In some embodiments, an antibody of the methods herein may be formulated in an arginine buffer. In one aspect, the arginine buffer may be an arginine succinate buffer. In one such aspect, the concentration of the arginine succinate buffer may be 50 mM or greater. In another such aspect, the concentration of the arginine succinate buffer may be 100 mM or greater. In another such aspect, the concentration of the arginine succinate buffer may be 150 mM or greater. In another such aspect, the concentration of the arginine succinate buffer may be 200 mM or greater. In another aspect, the arginine buffer formulation may further contain a surfactant. In another such aspect, the surfactant is a polysorbate. In another such aspect, the polysorbate is polysorbate 20. In another such aspect, the concentration of polysorbate 20 in the formulation is 0.1% or less. In another such aspect, the concentration of polysorbate 20 in the formulation is 0.05% or less. In another aspect, the pH of the arginine buffer formulation is between 4.5 and 7.0. In another aspect, the pH of the arginine buffer formulation is between 5.0 and 6.5. In another aspect, the pH of the arginine buffer formulation is between 5.0 and 6.0. In another aspect, the pH of the arginine buffer formulation is 5.5. In any of the foregoing embodiments and aspects, the antibody of the invention may be crenezumab.

In some embodiments, the formulation comprises about 100 mg/mL to about 300 mg/mL crenezumab. In some embodiments, the formulation comprises about 100 mg/mL, about 120 mg/mL, about 140 mg/mL, about 160 mg/mL, about 180 mg/mL, about 200 mg/mL, about 220 mg/mL, about 240 mg/mL, about 260 mg/mL, about 280 mg/mL, or about 300 mg/mL crenezumab.

In some embodiments, the formulation comprises 180 mg/mL crenezumab, 200 mM arginine succinate, 0.05% (w/v) polysorbate 20, wherein the formulation has a pH of 5.5.

In some embodiments, the methods herein comprise administration of a placebo. In some embodiments, the placebo comprises the same formulation as the anti-Aβ antibody, except for the antibody. For example, in an embodiment wherein the formulation comprises 180 mg/mL crenezumab, 200 mM arginine succinate, 0.05% (w/v) polysorbate 20, wherein the formulation has a pH of 5.5, the placebo would comprise a formulation comprising 200 mM arginine succinate, 0.05% (w/v) polysorbate 20, wherein the formulation has a pH of 5.5.

In some embodiments, the anti-Aβ antibody is formulated as a liquid for subcutaneous (SC) or intravenous (IV) administration.

### Routes of Administration

Dosing can be by any suitable route, e.g. by injections, such as intravenous or subcutaneous injections, depending in part on whether the administration is brief or chronic. In one embodiment, the antibody is injected subcutaneously. In another embodiment, the antibody is injected intravenously. In another embodiment, the antibody is administered using a syringe (e.g., prefilled or not) or an autoinjector.

In some embodiments, the anti-Aβ antibody is formulated for subcutaneous injection. In some embodiments, the anti-Aβ antibodies formulated for subcutaneous injection do not require dilution of the drug product prior to administration.

In some embodiments, the anti-Aβ antibody is administered by subcutaneous injection into the back of the arm. In some embodiments, the anti-Aβ antibody is administered by SC injection into the thigh. In some embodiments, the anti-Aβ antibody is administered by subcutaneous injection into the abdomen.

In some embodiments, the anti-Aβ antibody is administered subcutaneously in two injections (e.g., two injections of 360 mg for a total dose of 720 mg).

In some embodiments, the anti-Aβ antibody is administered by intravenous infusion.

### Dosing

For subcutaneous administration, about 360 mg to about 1000 mg of antibody can be an initial candidate dosage for administration to the patient whether, for example, by one or more separate administrations, or by continuous infusion. One typical daily, weekly, bi-weekly, monthly, or quarterly dosage might range from about 50 mg to about 4560 mg or more, depending on factors such as the patient's clinical history and response to the antibody and the discretion of the attending physician. The dosage can be administered in a single dose or a divided dose (e.g., two doses of 360 mg for a total dose of 720 mg). For repeated administrations over several weeks or longer, depending on the condition, the treatment would generally be sustained until a desired suppression of disease symptoms occurs. One exemplary dosage of the antibody would be in the range from about 540 mg to about 900 mg. In some embodiments, the total dose administered is in the range of 180 mg to 1440 mg. An exemplary dose of about 180 mg, about 200 mg, about 300 mg, about 360 mg, about 400 mg, about 500 mg, about 600 mg, about 700 mg, about 720 mg, about 800 mg, about 900 mg, about 1000 mg, about 1100 mg, about 1200 mg, about 1300 mg, about 1400 mg, or about 1440 mg (or any combination thereof) may be administered to the patient. Such doses may be administered intermittently, e.g. every week, every two weeks, every three weeks, every four weeks, every month, every two months, every three months, or every six months. However, other dosage regimens may be useful. The progress of this therapy can be monitored by conventional techniques and assays.

In certain embodiments, the antibody is administered subcutaneously at a dose of about 360 mg, about 400 mg, about 500 mg, about 540 mg, about 600 mg, about 700 mg, about 720 mg, about 800 mg, about 900 mg, about 1000 mg, about 1080 mg, or higher. In some embodiments, the antibody is administered subcutaneously at a dose of about 360 mg to about 400 mg, about 400 mg to about 500 mg, about 500 mg to about 600 mg, about 600 mg to about 700 mg, about 700 mg to about 720 mg, about 720 mg to about 800 mg, about 800 mg to about 900 mg, about 900 mg to about 1000 mg, about 1000 mg to about 1080 mg, about 360 to about 600 mg, about 500 mg to about 720 mg, about 700 mg to about 1000 mg, or about 720 mg to about 1080 mg. In some embodiments, the antibody is administered subcutaneously at a dose of about 720 mg or higher. In some embodiments, the antibody is delivered at a dose of about 300 mg. In some embodiments, the dose is administered subcutaneously every 2 weeks or every 4 weeks for a period of time. In certain embodiments, the period of time is 6 months, one year, eighteen months, two years, five years, ten years, 15 years, 20 years, or the lifetime of the patient.

In some embodiments, the antibody is administered in two subcutaneous injections at a dose of 360 mg each (720 mg total), with a total volume of 4.0 mL (2×2.0 mL) every two weeks. In some embodiments, the antibody is administered every two weeks over a period of at least 260 weeks. In some embodiments, the antibody is administered every two weeks over a period of at least 284 weeks. In some embodiments, the antibody is administered every two weeks for at least 10 years. In some embodiments, the antibody is administered every two weeks for at least 15 years. In some embodiments, the antibody is administered every two weeks for at least 20 years. In some embodiments, the antibody is administered every two weeks for the lifetime of the patient.

In some embodiments, a placebo is administered subcutaneously. In some embodiments, the placebo is administered in two subcutaneous injections for a total volume of 4.0 mL (2×2.0 mL) every two weeks. In some embodiments, the placebo is administered every two weeks over a period of at least 260 weeks.

For IV administration, about 15 mg/kg to about 200 mg/kg (e.g. 50 mg/kg-120 mg/kg, or any dosage within that range) of antibody can be an initial candidate dosage for administration to the patient, whether, for example, by one or more separate administrations, or by continuous infusion. One typical daily, weekly, bi-weekly, monthly, or quarterly dosage might range from about 15 mg/kg to 200 mg/kg or more, depending on factors such as the patient's clinical history and response to the antibody and the discretion of the attending physician. The dosage can be administered in a single dose or a divided dose (e.g., two doses of 30 mg/kg for a total dose of 60 mg/kg). For repeated administrations over several weeks or longer, depending on the condition, the treatment would generally be sustained until a desired suppression of disease symptoms occurs. One exemplary dosage of the antibody would be in the range from about 50 mg/kg to about 150 mg/kg. Thus, one or more doses of about 15 mg/kg, about 20 mg/kg, about 25 mg/kg, about 30 mg/kg, about 35 mg/kg, about 40 mg/kg, about 50 mg/kg, about 60 mg/kg, about 70 mg/kg, about 80 mg/kg, about 90 mg/kg, about 100 mg/kg, about 110 mg/kg, about 120 mg/kg, or about 130 mg/kg (or any combination thereof) may be administered to the patient. In some embodiments, one or more doses of about 60 mg/kg or higher may be administered to the patient. In some embodiments, the total dose administered is in the range of 1500 mg to 24000 mg. An exemplary dose of about 1500 mg, about 1600 mg, about 1700 mg, about 1800 mg, about 2000 mg, about 3000 mg, about 4000 mg, about 5000 mg, about 6000 mg, about 7000 mg, about 7200 mg, about 10000 mg, about 10500 mg, about 11000 mg, about 12000 mg, about 13000 mg, about 14000 mg, about 15000 mg, about 16000 mg, about 17000 mg, about 18000 mg, about 19000 mg, about 20000 mg, about 20500 mg, about 21000 mg, about 22000 mg, about 23000 mg, or about 24000 mg (or any combination thereof) may be administered to the patient. In some embodiments, the antibody is administered by IV at a) a dose of about 60 mg/kg or higher; or b) a fixed dose of 4200 mg or higher; or c) a fixed dose of about 4200 mg. Such doses may be administered intermittently, e.g. every week, every two weeks, every three weeks, every four weeks, every month, every two months, every three months, or every six months. However, other dosage regimens may be useful. The progress of this therapy can be monitored by conventional techniques and assays.

In certain embodiments, the antibody is administered by IV at a dose of about 15 mg/kg, about 20 mg/kg, about 30 mg/kg, about 40 mg/kg, about 45 mg/kg, about 50 mg/kg, about 60 mg/kg, about 70 mg/kg, about 80 mg/kg, about 90 mg/kg, about 100 mg/kg, about 110 mg/kg, about 120 mg/kg, about 130 mg/kg, about 140 mg/kg, about 150 mg/kg or a flat dose, e.g., about 1500 mg, about 1800 mg, about 2000 mg, about 2400 mg, about 3000 mg, about 3200 mg, about 4000 mg, about 5000 mg, about 5400 mg, about 6000 mg, about 7000 mg, about 7200 mg, about 8000 mg, or higher. In some embodiments, the antibody is administered by IV at a dose of about 15 mg/kg to about 20 mg/kg, about 20 mg/kg to about 30 mg/kg, about 30 mg/kg to about 40 mg/kg, about 40 mg/kg to about 45 mg/kg, about 45 mg/kg to about 50 mg/kg, about 50 mg/kg to about 60 mg/kg, about 60 mg/kg to about 70 mg/kg, about 70 mg/kg to about 80 mg/kg, about 80 mg/kg to about 90 mg/kg, about 90 mg/kg to about 100 mg/kg, about 100 mg/kg about 110 mg/kg, about 110 mg/kg to about 120 mg/kg, about 120 mg/kg to about 130 mg/kg, about 130 mg/kg to about 140 mg/kg, about 140 mg/kg to about 150 mg/kg or a flat dose, e.g., about 1500 mg to about 1800 mg, about 1800 mg to about 2000 mg, about 2000 mg to about 2400 mg, about 2400 mg to about 3000 mg, about 3000 mg to about 3200 mg, about 3200 mg to about 4000 mg, about 4000 mg to about 5000 mg, about 5000 mg to about 5400 mg, about 5400 mg to about 6000 mg, about 6000 mg to about 7000 mg, about 7000 mg to about 7200 mg, about 7200 mg to about 8000 mg. In some embodiments, the dose is administered by IV every 2 weeks or every 4 weeks for a period of time. In certain embodiments, the period of time is 6 months, one year, eighteen months, two years, five years, ten years, 15 years, 20 years, or the lifetime of the patient.

In some embodiments, the antibody is administered intravenously at a dose of 60 mg/kg every four weeks. In some embodiments, the antibody is administered every four weeks for at least 260 weeks. In some embodiments, the antibody is administered every four weeks for at least 5 years. In some embodiments, the antibody is administered every four weeks for at least 6 years. In some embodiments, the antibody is administered every four weeks for at least 7 years. In some embodiments, the antibody is administered every four weeks for at least 8 years. In some embodiments, the antibody is administered every four weeks for at least 9 years. In some embodiments, the antibody is administered every four weeks for at least 10 years. In some embodiments, the antibody is administered every four weeks for at least 11 years. In some embodiments, the antibody is administered every four weeks for at least 12 years. In some embodiments, the antibody is administered every four weeks for at least 13 years. In some embodiments, the antibody is administered every four weeks for at least 14 years. In some embodiments, the antibody is administered every four weeks for at least 15 years. In some embodiments, the antibody is administered every four weeks for at least 16 years. In some embodiments, the antibody is administered every four weeks for at least 17 years. In some embodiments, the antibody is administered every four weeks for at least 18 years. In some embodiments, the antibody is administered every four weeks for at least 19 years. In some embodiments, the antibody is administered every four weeks for at least 20 years. In some embodiments, the antibody is administered every four weeks for the lifetime of the patient.

In some embodiments, a placebo is administered intravenously. In some embodiments, the placebo is administered every four weeks for at least 260 weeks.

For IV dose calculations, the patient's weight is used. If the patient's weight changes by ≥10% from the previous reference weight, the current weight becomes the new reference weight for subsequent dosing. Additional recalculations are performed if a patient's weight further changes again ≥10%.

In some embodiments, a patient having an adverse effect to treatment may receive a reduced or modified dose of the drug.

### C. Therapeutic Responses and Assessments

In some embodiments, the methods provided herein result in one or more benefits to a human patient with a genetic mutation that causes familial AD. In some embodiments, the benefit (such as delayed symptom onset or slowed cognitive decline) is measured compared to a “reference” value. In some embodiments, a reference value is a measurement taken from one or more individuals with the same genetic mutation that have not received the anti-Aβ antibody. In some embodiments, the individuals that have not received the anti-Aβ antibody receive a placebo. In some embodiments, the reference value is a measurement taken from one or more individuals who do not have a genetic mutation associated with familial AD that have not received the anti-Aβ antibody. In some embodiments, the individuals that have not received the anti-Aβ antibody receive a placebo. In some embodiments, the reference is a measurement taken from a plurality of individuals comprising one or more individuals with the same genetic mutation as the patient receiving the anti-Aβ antibody and one or more individuals who do not have a genetic mutation associated with familial AD. In some embodiments, a population of human patients that receive the anti-Aβ antibody experiences AD progression, but to a lesser extent than a reference measure of progression obtained from individuals who did not receive the anti-Aβ antibody. In some embodiments, the human patient, or plurality of human patients who receive the anti-Aβ antibody have the same genetic mutation as individuals in the reference group.

In certain aspects, provided herein are methods of delaying onset of at least one symptom or slowing cognitive decline in a human patient with a genetic mutation that causes familial Alzheimer's Disease (AD), the methods comprising administering to the human patient an effective amount of a humanized monoclonal anti-amyloid beta to the human patient, wherein administering such a treatment to a plurality of human patients relative to a reference onset of at least one symptom or cognitive decline, wherein the reference onset of at least one symptom or cognitive decline is of a plurality of human patients who have received a placebo.

In some embodiments, administering such treatment results in a delay of onset of at least one symptom in the plurality of human patients relative to the reference onset of at least one symptom. In some embodiments, administering such treatment results in a delay in onset of at least one symptom relative to the reference onset of at least one symptom after about five years to about eight years of treatment. In some embodiments, the delay in onset of at least one symptom is statistically significant relative or compared to the reference onset of symptom. In some embodiments, administering such treatment results in a slowing of cognitive decline in the plurality of human patients relative to the reference cognitive decline. In some embodiments, administering such treatment results in a statistically significant slowing of cognitive decline in the plurality of human patients relative or compared to the reference cognitive decline after about five years to about eight years of treatment. In some embodiments, administering such treatment results in a reduction of cognitive impairment in the plurality of human patients relative to the reference cognitive impairment. In some embodiments, administering such treatment results in a reduction of cognitive impairment in the plurality of human patients relative or compared to the reference cognitive impairment about five years to about eight years after the start of treatment. In some embodiments, the reduction of cognitive impairment is statistically significant relative or compared to the reference. In some embodiments, administering such treatment results in a delayed onset in cognitive impairment relative or compared to the reference cognitive impairment.

In some embodiments, administering such treatment results in a delay of onset of at least one symptom in the plurality of human patients relative to the reference onset of at least one symptom. In some embodiments, administering such treatment results in a delay in onset of at least one symptom relative to the reference onset of at least one symptom after about five years to about eight years of treatment. In some embodiments, the delay in onset of at least one symptom is statistically significant relative or compared to the reference onset of at least one symptom. In some embodiments, administering such treatment results in a slowing of cognitive decline in the plurality of human patients relative to the reference cognitive decline. In some embodiments, administering such treatment results in a statistically significant slowing of cognitive decline in the plurality of human patients relative or compared to the reference cognitive decline after about five years to about eight years of treatment. In some embodiments, administering such treatment results in a reduction of cognitive impairment in the plurality of human patients relative to the reference cognitive impairment. In some embodiments, administering such treatment results in a reduction of cognitive impairment in the plurality of human patients relative or compared to the reference cognitive impairment about five years to about eight years after the start of treatment. In some embodiments, the reduction of cognitive impairment is statistically significant relative or compared to the reference.

In some embodiments, one or more cognitive, functional, and/or behavioral assessments is administered to measure the delay in at least one symptom, e.g., of AD or reduction in cognitive impairment. In some embodiments, the one or more assessments are administered only to the patient. In some embodiments, the one or more assessments are administered to a study partner. In some embodiments, the one or more assessments are administered to both the patient and the study partner. In some embodiments, the assessments are administered by one or more independent, blinded raters. In some embodiments, the independent, blinded rater has a psychometrician role. In some embodiments, the independent, blinded rater has a global rater role. In some embodiments, the assessments are administered by two or more independent blinded raters comprising at least one rater having a psychometrician role and at least one rater having a global rater role.

In some embodiments, the one or more assessments are not performed immediately after the patient is dosed with the antibody according to the methods herein. In some embodiments, the one or more assessments are not performed immediately after the patient undergoes a potentially stressful procedure (e.g., a blood draw or a procedure involving sedation). In some embodiments, the patient does not perform the one or more assessments while fasting.

In some embodiments, the one or more assessments are performed throughout the course of treatment. For example, the assessment may be performed every 1, 2, 4, 6, 8, 10, or every 12 weeks. In some embodiments, the assessment is not performed on a regular schedule, for example, the assessment may be performed at the discretion of the treating physician. In some embodiments, one or more assessments are performed on the first day of treatment. In some embodiments, the assessments performed on the first day of treatment are used as a baseline assessment from which subsequent assessments are compared.

In some embodiments, the one or more assessments given to a patient and/or a study partner are administered by the same psychometrician and/or the same global rater at each visit. In some embodiments, the one or more assessments are given by a study partner. A study partner is an individual who can report on cognitive changes in a patient and otherwise assist the patient in complying with the treatment regimen. In some embodiments, a Study Partner Characterization Questionnaire (SPCQ) is completed prior to the administration of the study partner assessments at each point in which a study partner assessment is performed. A SPCQ is a brief set of questions designed to identify the study partner, assess the patient's living arrangements (e.g., whether they live alone or with others), and assess the study partner's relationship with the participant.

In some embodiments, the delay in at least one symptom, reduction in cognitive decline, or prevention of cognitive impairment is measured according to the API ADAD Cognitive Composite Test Battery. The API ADAD Composite Cognitive Test Battery provides a composite cognitive test score designed to be highly sensitive to detecting and tracking preclinical cognitive decline, corresponding to an analysis of a change from baseline, rather than to optimize the discrimination between those who progress to clinical AD versus those who remain cognitively unimpaired (see, e.g., Ayutyanont et al., J. Clin. Psychiatry, 2014; 75 (6): 652-660, and Tariot et al., Alzheimers Dement., 2018; 4:150-160, which are hereby incorporated by reference in their entirety). The API ADAD Composite Cognitive Test Battery consists of the Mini-Mental State Exam (MMSE), Word List, Consortium to Establish a Registry for Alzheimer's Disease (CERAD) Constructional Praxis, Multilingual Naming Test, and Raven's Progressive Matrices. The MMSE evaluates orientation, attention, concentration, naming, repetition, comprehension, ability to create a sentence, and copy a figure (see, e.g., Folstein et al., J. Psychiatr. Res., 1975; 12:189-198, which is hereby incorporated by reference in its entirety); for calculation in the API ADAD Composite Test Battery, only the Orientation to Time score from MMSE is used. Word List is a measure of delayed verbal memory administered per the CERAD test protocol comprised of four parallel word lists derived from the ADAS-Cog word pool (see, e.g., Morris et al., Neurology, 1989; 39:1159-1165, which is hereby incorporated by reference in its entirety); for calculation in the API ADAD Composite Test Battery, only the Recall score is used. The CERAD Construction Praxis test has been widely used for evaluating cognitive deficits associated with Alzheimer's Disease and consists of four line drawings of increasing complexity (see, e.g., Morris et al., Neurology, 1989; 39:1159-1165, which is hereby incorporated by reference in its entirety); for purposes of the API ADAD Composite Test Battery, subjects are shown a figure and are then asked to immediately recall the figure. The Multilingual Naming Test is a measure of visual confrontation naming that requires the participant to name objects depicted in outline drawings graded by difficulty, with difficulty being based on frequency of occurrence (see, e.g., Gollan et al., Biling, 2012; 15:189-198, which is hereby incorporated by reference in its entirety). Raven's Progressive Matrices is a non-verbal, multiple choice measure of general ability and reasoning in the visual modality that requires conceptualization of special design and numerical relationships with each participant being asked to identify the missing component to complete a pattern (see, e.g., Raven, Raven Progressive Matrices and Vocabulary Scale, 1976, which is hereby incorporated by reference in its entirety).

In some embodiments, the API ADAD Cognitive Composite Test Battery is administered in the following order: MMSE, Multilingual Naming Test, Word List: Memory, CERAD Constructional Praxis, Word List: Recall, Word List: Recognition, Raven's Progressive Matrices (Set A).

In some embodiments, the API ADAD Cognitive Composite Test Battery is administered every 12 to 28 weeks during the course of treatment. In some embodiments, the API ADAD Cognitive Composite Test Battery is administered on weeks 1, 12, 24, 52, 76, 104, 128, 156, 180, 208, 232, and 260 for patients receiving the antibody by subcutaneous injection. In some embodiments, the API ADAD Cognitive Composite Test Battery is administered on weeks 1, 24, 36, 52, 76, 104, 128, 156, 180, 208, 232, and 260 for patients receiving the antibody intravenously. In some embodiments, the administration of the API ADAD Cognitive Composite Test Battery may continue every 24-28 weeks thereafter until treatment stops.

In some embodiments, administering such treatment results in a reduced decline on the API ADAD Cognitive Composite Test Battery in the plurality of human patients relative or compared to the reference API ADAD Cognitive Composite Test Battery about five years to about eight years after the start of treatment. In some embodiments, the reduced decline is statistically significant relative or compared to the reference. In some embodiments, administering such treatment results in an annualized rate of change of the API ADAD Composite Cognitive Test Battery. In some embodiments, administering such treatment results in a reduced annualized rate of change on the API ADAD composite Cognitive Test Battery relative or compared to the reference annualized rate of change on the API ADAD Cognitive Composite Test Battery.

In some embodiments, administering such treatment to the plurality of human patients results in a reduced annualized rate of change on an API ADAD Composite score of the plurality of human patients relative to a reference annualized rate of change on an API ADAD Composite score, and the reference annualized rate of change on an API ADAD Composite score is the annualized rate of change on an API ADAD Composite score of a plurality of human patients who have received the placebo. In some embodiments, the API ADAD Composite Cognitive Test Battery comprises a Word List Recall, Multilingual Naming Test, Mini-Mental State Examination (MMSE), CERAD Constructional praxis, and Raven's Progressive Matrices. In some embodiments, administering such treatment results in a reduced annualized rate of change in the API ADAD Composite score after treatment of about five years or longer. In some embodiments, administering such treatment results in a reduction of the annualized rate of change in the API ADAD Composite score in the plurality of human patients by at least 20%, at least 30%, at least 40%, at least 50%, about 20 to about 40%, or about 20%, about 21%, about 22%, about 23%, about 24%, about 25%, about 26%, about 27%, about 28%, about 29%, or about 30% relative to the reference annualized rate of change on an API ADAD Composite score. In some embodiments, administering such treatment results in a reduction of the annualized rate of change in the API ADAD Composite score in the plurality of human patients by at least about 20% relative to the reference annualized rate of change on an API ADAD Composite score. In some embodiments, administering such treatment results in a reduction of the annualized rate of change in the API ADAD Composite score in the plurality of human patients by at least about 30% relative to the reference annualized rate of change on an API ADAD Composite score. In some embodiments, administering such treatment results in a reduction of the annualized rate of change in the API ADAD Composite score in the plurality of human patients by 20% to 40% relative to the reference annualized rate of change on an API ADAD Composite score. In some embodiments, administering such treatment results in a reduction of the annualized rate of change in the API ADAD Composite score in the plurality of human patients by about 22% relative to the reference annualized rate of change on an API ADAD Composite score. In some embodiments, the administering such treatment to the plurality of human patients results in a reduced decline in the Free and Cued Selective Reminding Task (FCSRT) Cueing Index. The FCSRT provides assessment of immediate and delayed verbal episodic memory using Controlled Learning to optimize encoding specificity for more effective recall (see, e.g., Grober et al., Dev. Neuropsychol, 1987; 3:13-36 and Buschke, J. Clin. Neuropsychol, 1984; 6:433-440, which are hereby incorporated by reference in their entirety). For this test, participants are shown items on a card and are tasked to learn the items, attempt free recall of the items, followed by a semantically cued recall of items not produced by the participant during free recall. The FCSRT is thought to be sensitive to conditions, such as AD that compromise the functioning of the hippocampus and connected networks.

In some embodiments, administering such treatment results in a reduced decline of FCSRT Cueing Index in the plurality of human patients relative or compared to the reference decline FCSRT Cueing Index about five years to about eight years after the start of treatment. In some embodiments, the reduced decline is statistically significant relative or compared to the reference decline. In some embodiments, administering such treatment results in a reduced annualized rate of change on the FCSRT Cueing Index in the plurality of human patients relative or compared to the reference annualized rate of change on the FCSRT Cucing Index. In some embodiments, administering such treatment results in a statistically significant reduced annualized rate of change on the FCSRT Cueing Index in the plurality of human patients relative or compared to the reference annualized rate of change on the FCSRT Cueing Index about five years to about eight years after the start of treatment.

In some embodiments, administering such treatment results in a reduction of the annualized rate of change of the FCSRT Cueing Index in the plurality of human patients by at least 10%, at least 20%, at least 30%, at least 40%, about 10% to about 30%, or about 10%, about 11%, about 12%, about 13%, about 14%, about 15%, about 16%, about 17%, about 18%, about 19%, about 20%, about 21%, about 22%, about 23%, about 24%, or about 25%, about 26%, about 27%, about 28% or about 30% relative to the reference annualized rate of change on the FCSRT Cueing Index.

In some embodiments, administering such treatment results in a reduction of the annualized rate of change of the FCSRT Cueing Index in the plurality of human patients by at least about 10%. In some embodiments, administering such treatment results in a reduction of the annualized rate of change of the FCSRT Cueing Index in the plurality of human patients by at least about 20%. In some embodiments, administering such treatment results in a reduction of the annualized rate of change of the FCSRT Cueing Index in the plurality of human patients by about 10% to about 30%. In some embodiments, administering such treatment results in a reduction of the annualized rate of change of the FCSRT Cueing Index in the plurality of human patients by about 20% to about 30%

In some embodiments, the FCSRT Cueing Index is assessed using controlled learning.

In some embodiments, the FCSRT Cueing Index is administered every 12 to 28 weeks during the course of treatment. In some embodiments, the administration of the FCSRT Cueing Index may continue every 24-28 weeks thereafter until treatment stops.

In some embodiments, administering such treatment results in a statistically significant reduction in change over baseline in a cognitive measurement of the plurality of human patients relative or compared to a reference cognitive measurement, wherein the reference cognitive measurement is the cognitive measurement of a plurality of human patients who have received the placebo, wherein the cognitive measurement is selected from the group consisting of i) Trail Making Test, ii) Mini-Mental State Examination (MMSE), iii) Repeatable Battery for the Assessment of Neuropsychological Status (RBANS) Index Scores, iv) scores of each of the components of the API ADAD Composite Cognitive Test Battery, v) Preclinical Alzheimer's Cognitive Composite (PACC), and vi) other clinical endpoints.

The Trail Making Test comprises two parts and is described in Armitage, Psychological Monographs, 1946, 60:1-48, which is hereby incorporated by reference in its entirety. During Part A, participants are instructed to connect numbered circles with a line as quickly as possible in ascending numerical order. Part B uses a mix of circles that are either numbered or contain letters and participants must connect the circles while alternating between numbers and letters, in an ascending order. Performance is judged in terms of time to complete, the number of correctly connected circles, and the number of errors.

The RBANS is a standardized brief, individually administered neurocognitive battery measuring attention, language, visuospatial/constructional abilities, and immediate and delayed memory (see, e.g., Randolf, Repeatable Battery for the Assessment of Neuropsychological Status, 1998, which is hereby incorporated by reference in its entirety).

The Preclinical Alzheimer's Cognitive Composite (PACC) is a composite made up of different well established measures that assess cognitive function (see, e.g., Donohue et al., JAMA Neurology, 2014, 71:961-970, which is hereby incorporated by reference in its entirety). As used herein, the PACC will comprise the FCSRT free and cued recall, the MMSE, RBANs story recall, and the RBANs coding score.

In some embodiments, administering such treatment results in a reduction in change over baseline of i) the Trial Making Test, ii) the Mini-Mental State Examination (MMSE), iii) the Repeatable Battery for the Assessment of Neuropsychological Status (RBANS) Index Scores, iv) the scores of each of the components of the API ADAD Composite Cognitive Test Battery, v) the Preclinical Alzheimer's Cognitive Composite (PACC), and vi) other clinical endpoints.

In some embodiments, administering such treatment results in a statistically significant reduction in change over baseline in a Neuropsychiatric Inventory (NPI) of the plurality of human patients relative or compared to a reference NPI, wherein the reference NPI is the NPI of a plurality of human patients who have received the placebo. In some embodiments, administering such treatment results in a statistically significant reduction in change over baseline in NPI about five years to about eight years after the start of treatment.

The NPI is a well-validated, reliable, multi-item instrument to assess psychopathology in AD as well as the level of distress experienced by the informant in response to the presence of a given feature (see, e.g., Kaufer et al., J. Neuropsychiatry Clin. Neurosci., 2000; 12:233-239, Cummings et al., Neurology, 1994; 44:2308-2314, which are hereby incorporated by reference in their entirety). In some embodiments, the assessment is based on an interview with an informant and evaluates both the presence and severity and informant distress of neuropsychiatric features, including but not limited, delusions, hallucinations, dysphoria, anxiety, agitation/aggression, euphoria, disinhibition, irritability, lability, apathy, and aberrant motor behavior.

In some embodiments, administering such treatment results in a statistically significant reduction in change over baseline in the Functional Assessment Staging of Alzheimer's Disease (FAST) total score of the plurality of human patients relative or compared to a reference FAST total score, wherein the reference FAST total score is the FAST total score of a plurality of human patients who have received the placebo. In some embodiments, administering such treatment results in a statistically significant reduction in change over baseline in FAST total score about five years to about eight years after the start of treatment.

The FAST was developed for use with Alzheimer's disease (AD) patients to stage a patient's level of disability with respect to AD (Reisberg, Psychopharmacol. Bull., 1988; 24:653-659, which is hereby incorporated by reference in its entirety). The FAST is comprised of 7 major levels of functioning (from normal adult to severe AD) and is derived from Axis V of the Brief Cognitive Rating Scale (BCRS) (Reisberg et al., Psychopharmacol. Bull., 1983; 47:47-50, which is hereby incorporated by reference in its entirety), which itself is derived from the Global Deterioration Scale (Reisberg et al., Am. J. Psychiatry, 1982; 139:1136-1139, which is hereby incorporated by reference in its entirety). The stages and substages are, thus, designed to correlate with the Global Deterioration Scale global level of cognition and functional capacity measures (Sclan and Reisberg, Int. Psychogeriatr., 1992; 4:55-69, which is hereby incorporated by reference in its entirety).

In some embodiments, administering such treatment results in a statistically significant reduction in change over baseline in the Subject Memory Checklist (SMC) of the plurality of human patients relative or compared to a reference SMC, wherein the reference SMC is the SMC of a plurality of human patients who have received the placebo. In some embodiments, administering such treatment results in a statistically significant reduction in change over baseline in SMC score about five years to about eight years after the start of treatment.

The Subjective Memory Checklist (SMC) is an assessment developed to collect subject ratings of memory status from both a participant and an informant. Participants and informants are asked to rate how often the given participant has difficulties relating to memory and thinking in a variety of areas. The SMC has a study partner portion and a participant portion. In some embodiments, the average Subjective Memory Checklist Score is greater than 22 and based on the average of participant and study partner component scores.

In some embodiments, administering such treatment results in a statistically significant reduction in change over baseline in the Geriatric Depression Scale (GDS) of the plurality of human patients relative or compared to a reference GDS, wherein the reference GDS is the GDS of a plurality of human patients who have received the placebo. In some embodiments, administering such treatment results in a statistically significant reduction in change over baseline in the GDS score about five years to about eight years after the start of treatment.

The Geriatric Depression Scale (GDS) is a scale designed to identify symptoms of depression in the elderly (Sheikh and Yesavage, Clinical Gerontologist, 1986, 5:165-173, which is hereby incorporated by reference in its entirety). The scale consists of 15 questions the participant is asked to answer on the basis of how they felt over the past week, with one point given for each answer indicative of a symptom of depression. Total scores of 0-4 are considered normal, scores of 5-8 suggest mild depression, scores of 9-11 suggest moderate depression, and scores of 12-15 suggest severe depression.

In some embodiments, administering such treatment results in a reduced Standardized Uptake Value Ratio (SUVR) of amyloid PET measurement in the plurality of human patients as compared to a reference SUVR of amyloid PET, wherein the reference SUVR of amyloid PET is a SUVR of amyloid PET of the plurality of human patients who have received the placebo. In some embodiments, administering such treatment results in a reduced Standardized Uptake Value Ratio (SUVR) of amyloid PET measurement in the plurality of human patients by at least 3% as compared to the reference SUVR of amyloid PET. In some embodiments, administering such treatment results in a reduced Standardized Uptake Value Ratio (SUVR) of amyloid PET measurement in the plurality of human patients by at least 10% as compared to the reference SUVR of amyloid PET. In some embodiments, administering such treatment results in a reduced Standardized Uptake Value Ratio (SUVR) of amyloid PET measurement in the plurality of human patients by about 3% to about 10% as compared to the reference SUVR of amyloid PET. In some embodiments, administering such treatment results in a reduced Standardized Uptake Value Ratio (SUVR) of amyloid PET measurement in the plurality of human patients by about 3%, about 4%, about 5%, about 6%, about 7%, about 8%, about 9%, or about 10% as compared to the reference SUVR of amyloid PET. In some embodiments, administering such treatment results in a reduced Standardized Uptake Value Ratio (SUVR) of amyloid PET measurement in the plurality of human patients by about 3% as compared to the reference SUVR of amyloid PET.

In some embodiments, administering such treatment to the plurality of human patients results in an increased time to progression from preclinical AD to mild cognitive impairment due to AD or from preclinical AD to dementia due to AD in the plurality of human patients relative to a reference time to progression from preclinical AD to mild cognitive impairment due to AD or from preclinical AD to dementia due to AD, wherein the reference time to progression from preclinical AD to mild cognitive impairment due to AD or from preclinical AD to dementia due to AD is the time to progression of a plurality of human patients who have received placebo. In some embodiments, administering such treatment results in an increased time to progression from preclinical AD to mild cognitive impairment due to AD or from preclinical AD to dementia due to AD in the plurality of human patients relative to a reference time to progression from preclinical AD to mild cognitive impairment due to AD or from preclinical AD to dementia due to AD about five years to about eight years after the start of treatment. In some embodiments, the increased time to progression is statistically significant relative or compared to the reference.

In some embodiments, administering such treatment to the plurality of human patients results in an increased time to progression from preclinical AD to dementia due to AD in the plurality of human patients by about 10% to about 30% relative to a reference time to progression from preclinical AD to dementia due to AD. In some embodiments, administering such treatment to the plurality of human patients results in an increased time to progression from preclinical AD to dementia due to AD in the plurality of human patients by at least about 10% relative to a reference time to progression from preclinical AD to dementia due to AD. In some embodiments, administering such treatment to the plurality of human patients results in an increased time to progression from preclinical AD to dementia due to AD in the plurality of human patients by at least about 20% relative to a reference time to progression from preclinical AD to dementia due to AD.

In some embodiments, administering such treatment to the plurality of human patients results in an increased time to progression from preclinical AD to mild cognitive impairment due to AD in the plurality of human patients by about 10% to about 30% relative to a reference time to progression from preclinical AD to mild cognitive impairment due to AD. In some embodiments, administering such treatment to the plurality of human patients results in an increased time to progression from preclinical AD to mild cognitive impairment due to AD in the plurality of human patients by at least about 10% relative to a reference time to progression from preclinical AD to mild cognitive impairment due to. In some embodiments, administering such treatment to the plurality of human patients results in an increased time to progression from preclinical AD to mild cognitive impairment due to AD in the plurality of human patients by at least about 20% relative to a reference time to progression from preclinical AD to mild cognitive impairment due to AD.

In some embodiments, administering such treatment to the plurality of human patients results in an increased time to progression from preclinical AD to dementia due to AD in the plurality of human patients by about 15%, about 16%, about 17%, about 18%, about 19%, about 20%, about 21%, about 22%, about 23%, about 24% or about 25% relative to a reference time to progression from preclinical AD to dementia due to AD. In some embodiments, administering such treatment to the plurality of human patients results in an increased time to progression from preclinical AD to dementia due to AD in the plurality of human patients by about 21%, relative to a reference time to progression from preclinical AD to dementia due to AD.

In some embodiments, administering such treatment to the plurality of human patients results in an increased time to progression from preclinical AD to mild cognitive impairment due to AD in the plurality of human patients by about 15%, about 16%, about 17%, about 18%, about 19%, about 20%, about 21%, about 22%, about 23%, about 24% or about 25% relative to a reference time to progression from preclinical AD to mild cognitive impairment due to AD. In some embodiments, administering such treatment to the plurality of human patients results in an increased time to progression from preclinical AD to mild cognitive impairment due to AD in the plurality of human patients by about 21%, relative to a reference time to progression from preclinical AD to mild cognitive impairment due to AD.

In some embodiments, progression to mild cognitive impairment (MCI) is assessed every 12-28 weeks during the course of treatment. In some embodiments, progression to MCI is assessed every 12 to 28 weeks during the course of treatment. In some embodiments, progression to MCI is assessed on weeks 1, 12, 24, 52, 76, 104, 128, 156, 180, 208, 232, and 260 for patients receiving the antibody by subcutaneous injection. In some embodiments, progression to MCI is assessed on weeks 1, 24, 36, 52, 76, 104, 128, 156, 180, 208, 232, and 260 for patients receiving the antibody intravenously. In some embodiments, progression to MCI is assessed every 24-28 weeks thereafter until treatment stops.

In some embodiments, progression to dementia due to AD is assessed every 12-28 weeks during the course of treatment. In some embodiments, progression to dementia due to AD is assessed every 12 to 28 weeks during the course of treatment. In some embodiments, progression to dementia due to AD assessed on weeks 1, 12, 24, 52, 76, 104, 128, 156, 180, 208, 232, and 260 for patients receiving the antibody by subcutaneous injection. In some embodiments, progression to dementia due to AD is assessed on weeks 1, 24, 36, 52, 76, 104, 128, 156, 180, 208, 232, and 260 for patients receiving the antibody intravenously. In some embodiments, progression to dementia due to AD is assessed every 24-28 weeks thereafter until treatment stops.

In some embodiments, progression to mild cognitive impairment (MCI) is determined based on cognitive concern in the judgment of the physician, based in part on the average Subjective Memory Checklist (SMC) score. In some embodiments, the SMC assessment is performed every 12-28 weeks during the course of treatment. In some embodiments, the SMC assessment is administered every 12 to 28 weeks during the course of treatment. In some embodiments, the SMC assessment is administered on weeks 1, 12, 24, 52, 76, 104, 128, 156, 180, 208, 232, and 260 for patients receiving the antibody by subcutaneous injection. In some embodiments, the SMC assessment is administered on weeks 1, 24, 36, 52, 76, 104, 128, 156, 180, 208, 232, and 260 for patients receiving the antibody intravenously. In some embodiments, the administration of the SMC assessment may continue every 24-28 weeks thereafter until treatment stops.

In some embodiments, MCI is a stage of dementia that is less severe than dementia. In some embodiments, MCI comprises decline in cognitive abilities such as language, memory reasoning, judgment, or perception that is not due to normal aging. Individuals in the MCI stage of severity can independently drive, shop, cook, pay bills, manage finances, do household chores and other well-learned skills that do not place significant demands upon learning new information. The MCI stage is not seen in normal aging individuals. and is due to one or more cognitive disorders. In Alzheimer's disease, the MCI stage lasts an average of 7 years.

In some embodiments, progression to MCI is determined based on objective evidence of impairment in the judgment of the physician in one or more cognitive domains on the basis of review of Clinical Dementia Rating (CDR), Word List (Recall), MMSE, and Repeatable Battery for the Assessment of Neuropsychological Status (RBANS). The physician may further elect to review and other clinical and cognitive test results in rendering this opinion except for the API ADAD Composite Cognitive Test total score or the CERAD total score.

The CDR describes five degrees of impairment in performance on each of six categories of cognitive function, including memory, orientation, judgment and problem solving, community affairs, home and hobbies, and personal care (see, e.g., Morris, Neurology, 1993; 43:2412-2413, which is hereby incorporated by reference in its entirety). The ratings of degree of impairment obtained on each of the six categories of function are synthesized into one global rating of dementia, where a score of 0 indicates no dementia, a score of 0.5 signifies MCI, and scores of 1, 2, or 3 refer to progressively more severe dementia (See, e.g., Morris 1993 and Morris et. al., Arch. Neurol., 58 (3): 397-405, 2001). Use of the CDR Sum of Boxes provides a more refined measure of change (Berg et al., Ann. Neurol., 1992; 31:242-249, which is hereby incorporated by reference in its entirety).

In some embodiments, the CDR assessment is performed every 12-28 weeks during the course of treatment. In some embodiments, the CDR assessment is administered every 12 to 28 weeks during the course of treatment. In some embodiments, the CDR assessment is administered on weeks 1, 12, 24, 52, 76, 104, 128, 156, 180, 208, 232, and 260 for patients receiving the antibody by subcutaneous injection. In some embodiments, the CDR assessment is administered on weeks 1, 24, 36, 52, 76, 104, 128, 156, 180, 208, 232, and 260 for patients receiving the antibody intravenously. In some embodiments, the administration of the CDR assessment may continue every 24-28 weeks thereafter until treatment stops. In some embodiments, progression to MCI is determined based on preservation of functional abilities in the judgment of the physician, based in part on review of the Functional Assessment Staging Test (FAST). In some embodiments, the FAST assessment is performed every 12-28 weeks during the course of treatment. In some embodiments, the FAST assessment is administered every 12 to 28 weeks during the course of treatment. In some embodiments, the FAST assessment is administered on weeks 1, 12, 24, 52, 76, 104, 128, 156, 180, 208, 232, and 260 for patients receiving the antibody by subcutaneous injection. In some embodiments, the FAST assessment is administered on weeks 1, 24, 36, 52, 76, 104, 128, 156, 180, 208, 232, and 260 for patients receiving the antibody intravenously. In some embodiments, the administration of the FAST assessment may continue every 24-28 weeks thereafter until treatment stops.

In some embodiments, progression to MCI requires cognitive concern in the judgment of the physician, based in part on the average Subjective Memory Checklist (SMC) score, objective evidence of impairment in the judgment of the physician in one or more cognitive domains on the basis of review of CDR, Word List (Recall), MMSE, and RBANS, preservation of functional abilities in the judgment of the physician, based in part on review of the FAST, documentation that MCI criteria have been met, and agreement by an external adjudication committee that the progression to MCI criteria have been met.

In some embodiments, progression to AD dementia is determined by physician review of medical history, interview of the participant and study party that establishes significant deterioration in the participant's cognitive and functional status since baseline, and review of CDR, Word List (Recall), MMSE total, RBANs, and Neuropsychiatric Inventory Questionnaire (NPI). The physician may further elect to review and other clinical and cognitive test result in rendering this opinion except for the API ADAD Composite Cognitive Test total score or the CERAD total score. A determination of progression to AD dementia further requires that dementia criteria are met and an agreement by an external adjudication committee that the progression to AD dementia have been met.

In some embodiments, the NPI assessment is performed every 12-28 weeks during the course of treatment. In some embodiments, the NPI assessment is administered every 12 to 28 weeks during the course of treatment. In some embodiments, the NPI assessment is administered on weeks 1, 12, 24, 52, 76, 104, 128, 156, 180, 208, 232, and 260 for patients receiving the antibody by subcutaneous injection. In some embodiments, the NPI assessment is administered on weeks 1, 24, 36, 52, 76, 104, 128, 156, 180, 208, 232, and 260 for patients receiving the antibody intravenously. In some embodiments, the administration of the NPI assessment may continue every 24-28 weeks thereafter until treatment stops.

In some embodiments, administering the treatment to the plurality of human patients results in an increased time to progression to a non-zero score in the Clinical Dementia Rating (CDR) Scale global score of the plurality of human patients. In some embodiments, administering the treatment results in an increased time to progression to a non-zero score in the CDR Scale global score compared of the plurality of human patients relative or compared to a reference CDR Scale global score about five years to about eight years after the start of treatment, wherein the reference CDR Scale global score is the CDR Scale global score of a plurality of human patients who have received the placebo. In some embodiments, the increased time to progression to a non-zero score is statistically significant relative or compared to the reference. In some embodiments, the CDR Scale global score describes impairment in memory, orientation, judgment and problem solving, community affairs, home and hobbies, and personal care.

In some embodiments, administering such treatment results in an increased time to progression to non-zero in the CDR Scale global score in the plurality of human patients by about 5% to about 20% relative to a reference time to progression to progression to non-zero in the CDR Scale global score. In some embodiments, administering such treatment results in an increased time to progression to non-zero in the CDR Scale global score in the plurality of human patients by at least about 5% relative to a reference time to progression to progression to non-zero in the CDR Scale global score. In some embodiments, administering such treatment results in an increased time to progression to non-zero in the CDR Scale global score in the plurality of human patients by at least about 10% relative to a reference time to progression to progression to non-zero in the CDR Scale global score. In some embodiments, administering such treatment results in an increased time to progression to non-zero in the CDR Scale global score in the plurality of human patients by about 5%, about 6%, about 7%, about 8%, about 9%, about 10%, about 11%, about 12% about 13%, about 14%, about 15%, about 16%, about 17%, about 18%, about 19% or about 20% relative to a reference time to progression to progression to non-zero in the CDR Scale global score. In some embodiments, administering such treatment results in an increased time to progression to non-zero in the CDR Scale global score in the plurality of human patients by 8% relative to a reference time to progression to progression to non-zero in the CDR Scale global score. In some embodiments, administering the treatment to the plurality of human patients results in a reduced decline in the Clinical Dementia Rating (CDR) Scale global score of the plurality of human patients. In some embodiments, administering the treatment results in a reduced decline in the CDR Scale global score compared of the plurality of human patients relative or compared to a reference CDR Scale global score about five years to about eight years after the start of treatment, wherein the reference CDR Scale global score is the CDR Scale global score of a plurality of human patients who have received the placebo. In some embodiments, the reduced decline is statistically significant relative or compared to the reference. In some embodiments, the CDR Scale global score describes impairment in memory, orientation, judgment and problem solving, community affairs, home and hobbies, and personal care.

In some embodiments, administering such treatment to the plurality of human patients results in a reduced decline on a Clinical Dementia Rating (CDR) Scale Sum of Boxes of the plurality of human patients relative to a reference decline on a CDR Scale Sum of Boxes, wherein the reference decline on a CDR Scale Sum of Boxes is the decline on a CDR Scale Sum of Boxes of a plurality of human patients who have received placebo. In some embodiments, administering such treatment results in a statically significant reduced decline on a CDR Scale Sum of Boxes of the plurality of human subjects relative or compared to the reference decline on a CDR Scale Sum of Boxes about five years to about eight years after the start of treatment.

In some embodiments, administering such treatment to the plurality of human patients results in a reduced decline on a Clinical Dementia Rating (CDR) Scale Sum of Boxes global score of the plurality of human patients relative to a reference decline on a CDR Scale Sum of Boxes global, wherein the reference decline on a CDR Scale Sum of Boxes global score is the decline on a CDR Scale Sum of Boxes global score of a plurality of human patients who have received placebo.

In some embodiments, administering such treatment to the plurality of human patients results in a reduced decline in the annualized rate of change on a Clinical Dementia Rating (CDR) Scale Sum of Boxes of the plurality of human patients relative to a reference annualized rate of change on a CDR Scale Sum of Boxes, wherein the reference annualized rate of change on a CDR Scale Sum of Boxes is the annualized rate of change on a CDR Scale Sum of Boxes of a plurality of human patients who have received placebo. In some embodiments, administering such treatment results in a statically significant reduced decline in the annualized rate of change on a CDR Scale Sum of Boxes of the plurality of human subjects relative or compared to the reference annualized rate of change on a CDR Scale Sum of Boxes about five years to about eight years after the start of treatment. In some embodiments, administering such treatment results in a reduced annualized rate of change on a CDR Scale Sum of Boxes global score in the plurality of human patients by at least 5% relative to a reference CDR Scale Sum of Boxes global score. In some embodiments, administering such treatment results in a reduced annualized rate of change on a CDR Scale Sum of Boxes global score in the plurality of human patients by at least 10% relative to a reference CDR Scale Sum of Boxes global score. In some embodiments, administering such treatment results in a reduced annualized rate of change on a CDR Scale Sum of Boxes global score in the plurality of human patients by about 5% to about 20% relative to a reference CDR Scale Sum of Boxes global score. In some embodiments, administering such treatment results in a reduced annualized rate of change on a CDR Scale Sum of Boxes global score in the plurality of human patients by about 3%, about 5%, about 5%, about 6% about 7% about 8%, about 9%, about 10%, about 15%, or about 20% relative to a reference CDR Scale Sum of Boxes global score. In some embodiments, administering such treatment results in a reduced annualized rate of change on a CDR Scale Sum of Boxes global score in the plurality of human patients by about 9% relative to a reference CDR Scale Sum of Boxes global score.

In some embodiments, administering such treatment to the plurality of human patients results in a reduced annualized rate of change in a measure of overall neurocognitive functioning of the plurality of human patients relative to a reference annualized rate of change in a measure of overall neurocognitive functioning wherein the reference annualized rate of change in a measure of overall neurocognitive functioning is the annualized rate of change in a measure of overall neurocognitive functioning of the plurality of human patients who have received placebo. In some embodiments, the annualized rate of change in a measure of overall neurocognitive functioning is determined using a Repeatable Battery for the Assessment of Neuropsychological Status (RBANS) score. In some embodiments, administering such treatment results in a statistically significant reduction in an annualized rate of change of the RBANS score relative or compared to the reference annualized rate of change of RBANS score about five years to about eight years after the start of treatment.

In some embodiments, the annualized rate of change in a measure of overall neurocognitive functioning is determined using a Repeatable Battery for the Assessment of Neuropsychological Status (RBANS) score. In some embodiments, administering such treatment results in a numerically favorable reduction in an annualized rate of change of the RBANS score relative or compared to the reference annualized rate of change of RBANS score. In some embodiments, administering such treatment results in reduction of the RBANS score by at least 40% relative to the reference RBANS score. In some embodiments, administering such treatment results in reduction of the RBANS score by at least 50% relative to the reference RBANS score. In some embodiments, administering such treatment results in reduction of the RBANS score by about 30% to about 60% relative to the reference RBANS score. In some embodiments, administering such treatment results in reduction of the RBANS score by about 40% to about 50% relative to the reference RBANS score. In some embodiments, administering such treatment results in reduction of the RBANS score by about 30%, about 31%, about 32% about 33%, about 34%, about 35%, about 36%, about 37%, about 38%, about 39%, about 40% about 41% about 42% about 43%, about 44%, about 45%, about 46%, about 47%, about 50%, about 55%, or about 60% relative to the reference RBANS score. In some embodiments, administering such treatment results in reduction of the RBANS score by about 43% relative to the reference RBANS score.

### Biomarkers

In some embodiments, the methods herein further result in an effect on a biomarker associated with MCI and/or AD. In some embodiments, the effect on the biomarker is compared to a reference level of the biomarker. In some embodiments, a biomarker associated with MCI and/or AD includes CDR, Word List: Recall, MMSE total, RBANS, FAST, and/or NPI. In some embodiments, the effect on the biomarker is compared to a baseline level of the biomarker established at the beginning of treatment. In some embodiments, the effect on the biomarker is compared to the baseline level of the biomarker. In some embodiments, the effect on the biomarker is observed about 12 weeks after the start of treatment. In some embodiments, the effect on the biomarker is observed about 5 years after the start of treatment. In some embodiments, the effect on the biomarker is observed more than 5 years after the start of treatment, such as about 6, about 7, about 8, about 9, about 10, about 15, or about 20 or more years after the start of treatment.

In some embodiments, the reference level is based on the level of the biomarker in a patient population receiving a placebo.

In some embodiments, the biomarker is an imaging biomarker, such as a biomarker that is assessed using positron emission tomography (PET) or magnetic resonance imaging (MRI). In some embodiments, the biomarker is assessed using an immunoassay. In some embodiments, the biomarker is assessed using ELISA (enzyme-linked immunosorbent assay).

Brain amyloid load or burden, neurofibrillary tangles, and/or regional cerebral metabolic rate of glucose (CMRgI) can be assessed using neurological imaging techniques and tools, for example using PET (positron emission tomography) scanning. Serial PET scans of a patient taken over time, e.g., before and after administration of a treatment (or at one or more intervals throughout the course of a treatment regimen), can permit detection of increased, decreased, or unchanged amyloid burden, neurofibrillary tangles, and/or glucose metabolism in the brain. This technique can further be used to determine whether these biomarkers are increasing or decreasing.

Tau levels, or Tau load, in a patient, can be assessed using neurological imaging techniques and tools, for example using PET (positron emission tomography) scanning. In such methods, a tracer molecule known to bind to Tau is radiolabeled with a PET-sensitive radioisotope and introduced into the patient. In conjunction with scans of the patient's brain, the location and quantity (i.e., distribution) of Tau can be imaged based on the assumption that the tracer binds to the Tau molecules. Serial PET scans of a patient taken over time, e.g., before and after administration of a treatment (or at one or more intervals throughout the course of a treatment regimen), can permit detection of increased, decreased, or unchanged Tau load in the patient's brain. Tau levels in a patient also can be assessed by measuring Tau in a blood, serum, plasma, or CSF sample from the patient. One or more of these techniques can be used to determine whether total Tau is increasing or decreasing, or whether a given isoform of Tau (e.g., aggregated Tau) is increasing or decreasing, at various time points over the course of treatment.

In some embodiments, the Tau tracer is [18F] Genentech Tau Probe 1 ([18F] GTP1), as described in U.S. Pat. No. 10,076,581. In other embodiments, other Tau Probes can be used. Examples of such tracer molecules include but are not limited to: RO-948 (F. Hoffmann-La Roche AG); AV-1451 (“Flortaucepir”, Avid, Inc.); PI-2014, and PI-2620 (AC Immune); MK-6240 (Merck Sharp & Dohme); and T-808 (Eli Lilly & Co.).

Methods of quantifying the Tau distribution in a patient's brain, based on imaging a radio-labeled tracer, include “Standardized Uptake Value Ratio” (SUVR) (see, e.g., J. Nucl. Med., S. Sanabria Bohorquez et al., 58 (1), (2017), incorporated herein by reference).

Tau imaging using a radioligand such as [18F] GTP1 provides advantages over conventional CSF biomarker evaluation in that it allows for the relationship between the distribution of Tau pathology and response to anti-Tau therapy to be evaluated. For example, longitudinal [18F] GTP1 PET imaging data can be collected to assess the response of this biomarker as done with, e.g., semorinemab, as it has the potential to inform the relationship between spatial distribution of Tau pathology, cognitive function, and disease progression.

In some embodiments, administering such treatment according to the methods herein results in an effect on a tau-based cerebrospinal fluid (CSF) biomarker relative or compared to a reference tau-based CSF biomarker, wherein the tau-based CSF biomarker is the tau-based CSF biomarker of a plurality of human patients who have received the placebo. Tau is a microtubule associated protein that forms insoluble filaments that accumulate as neurofibrillary tangles in AD patients. In some embodiments, the tau-based CSF biomarker is detected using the elecsys assay by Roche Diagnostics. In some embodiments, the tau-based CSF biomarker is detected using a fluorine labeled probe. In some embodiments the fluorine label comprises fluorine-18 (18F). In some embodiments, the probe is 18F GTP1 (see, e.g., Bohorquez et al., Eur. J. Nucl. Med. Mol. Imaging, 2019; 46 (10): 2077-2089). In some embodiments, the tau-based CSF biomarker is measured using positron emission tomography (PET). In some embodiments, administering such treatment results in a statistically significant change in the tau-based CSF biomarker in the plurality of human patients relative or compared to the reference tau-based CSF biomarker about 12 weeks after the start of treatment. In some embodiments, administering such treatment results in a statistically significant change in the tau-based CSF biomarker in the plurality of human patients relative or compared to the reference tau-based CSF biomarker about five years to about eight years after the start of treatment. In some embodiments, administering the treatment according to the methods herein results in a decrease in the tau-based CSF biomarker relative or compared to the reference tau-based CSF biomarker.

In some embodiments, administering such treatment results in a reduction of annualized rate of change in the tau-based CSF biomarker in the plurality of human patients compared to a reference annualized rate of change in the tau-based CSF biomarker, wherein the reference annualized rate of change in the tau-based CSF biomarker is the annualized rate of change in the tau-based CSF biomarker of a plurality of human patients who have received the placebo.

In some embodiments, administering such treatment results in a reduction of annualized rate of a tau-based CSF biomarker in the plurality of human patients relative to the reference tau-based CSF biomarker, said tau-based CSF biomarker being a phospho-tau [ptau]-based CSF biomarker. In some embodiments, administering such treatment results in a reduction of annualized rate of the tau-based CSF biomarker in the plurality of human patients by at least 40% relative to the reference tau-based CSF biomarker, said tau-based CSF biomarker being a phospho-tau [ptau]-based CSF biomarker. In some embodiments, administering such treatment results in a reduction of annualized rate of the tau-based CSF biomarker in the plurality of human patients by at least 30% relative to the reference tau-based CSF biomarker, said tau-based CSF biomarker being a phospho-tau [ptau]-based CSF biomarker. In some embodiments, administering such treatment results in a reduction of annualized rate of the tau-based CSF biomarker in the plurality of human patients by 30% to 50% relative to the reference tau-based CSF biomarker, said tau-based CSF biomarker being a phospho-tau [ptau]-based CSF biomarker. In some embodiments, administering such treatment results in a reduction of annualized rate of the tau-based CSF biomarker in the plurality of human patients by about 30%, about 31%, about 32%, about 33%, about 34%, about 35%, about 36%, about 37%, about 38%, about 39%, about 40%, about 45%, or about 50% relative to the reference tau-based CSF biomarker, said tau-based CSF biomarker being a phospho-tau [ptau]-based CSF biomarker. In some embodiments, administering such treatment results in a reduction of annualized rate of the tau-based CSF biomarker in the plurality of human patients by about 37% relative to the reference tau-based CSF biomarker, said tau-based CSF biomarker being a phospho-tau [ptau]-based CSF biomarker.

In some embodiments, administering such treatment results in a reduction of annualized rate of a tau-based CSF biomarker in the plurality of human patients relative to the reference tau-based CSF biomarker, said tau-based CSF biomarker being a total-tau [ttau]-based CSF biomarker. In some embodiments, administering such treatment results in a reduction of annualized rate of a tau-based CSF biomarker in the plurality of human patients by at least about 20% relative to the reference tau-based CSF biomarker, said tau-based CSF biomarker being a total-tau [ttau]-based CSF biomarker. In some embodiments, administering such treatment results in a reduction of annualized rate of a tau-based CSF biomarker in the plurality of human patients by at least about 30% relative to the reference tau-based CSF biomarker, said tau-based CSF biomarker being a total-tau [ttau]-based CSF biomarker. In some embodiments, administering such treatment results in a reduction of annualized rate of a tau-based CSF biomarker in the plurality of human patients by about 20% to about 40% relative to the reference tau-based CSF biomarker, said tau-based CSF biomarker being a total-tau [ttau]-based CSF biomarker. In some embodiments, administering such treatment results in a reduction of annualized rate of a tau-based CSF biomarker in the plurality of human patients by about 20%, about 21%, about 22%, about 23%, about 24%, about 25%, about 26%, about 27%, about 28%, about 29%, or about 30% relative to the reference tau-based CSF biomarker, said tau-based CSF biomarker being a total-tau [ttau]-based CSF biomarker. In some embodiments, administering such treatment results in a reduction of annualized rate of a tau-based CSF biomarker in the plurality of human patients by about 28% relative to the reference tau-based CSF biomarker, said tau-based CSF biomarker being a total-tau [ttau]-based CSF biomarker.

In some embodiments, administering such treatment to the plurality of human patients results in an effect on a brain tau load compared to a reference a brain tau load and the reference brain tau load is a brain tau load of a plurality of human patients who have received the placebo. In some embodiments, the brain tau load is measured using positron emission tomography (tau-PET). In some embodiments, administering such treatment results in a reduction of annualized rate of change in a tau-PET measurement in the plurality of human patients relative to a reference tau-PET, wherein the reference tau-PET measurement is a tau-PET measurement of a plurality of human patients who have received the placebo. In some embodiments, the brain tau load is detected using a fluorine labeled probe. In some embodiments the fluorine label comprises fluorine-18 (18F). In some embodiments, the probe is 18F GTP1 (scc, e.g., Bohorquez et al., Eur. J. Nucl. Med. Mol. Imaging. 2019; 46 (10): 2077-2089).

In some embodiments, administering such treatment to the plurality of human patients results in an numerically favored effect of the Tau-PET compared to a reference a brain Tau-PET and the reference brain Tau-PET is a brain Tau-PET of a plurality of human patients who have received the placebo. In some embodiments, administering such treatment to the plurality of human patients results in a reduction of annualized rate of the tau-PET in the plurality of human patients by about 40% to about 60% relative to the reference tau-PET. In some embodiments, administering such treatment to the plurality of human patients results in a reduction of annualized rate of the tau-PET in the plurality of human patients by about 50% to about 60% relative to the reference tau-PET. In some embodiments, administering such treatment to the plurality of human patients results in a reduction of annualized rate of the tau-PET in the plurality of human patients by at least 40% relative to the reference tau-PET. In some embodiments, administering such treatment to the plurality of human patients results in a reduction of annualized rate of the tau-PET in the plurality of human patients by at least 50% relative to the reference tau-PET. In some embodiments, administering such treatment to the plurality of human patients results in a reduction of annualized rate of the tau-PET in the plurality of human patients by about 40%, about 41%, about 42%, about 43%, about 44%, about 45%, about 46%, about 47%, about 47%, about 48%, about 49%, about 50%, about 51%, about 52%, about 53%, about 54%, about 55%, about 56%, about 57%, about 58%, about 59%, or about 60% relative to the reference tau-PET. In some embodiments, administering such treatment to the plurality of human patients results in a reduction of annualized rate of the tau-PET in the plurality of human patients by about 51% relative to the reference tau-PET.

In some embodiments, administering such treatment according to the methods herein results in a reduction of cerebral fibrillary amyloid burden in a predefined region of interest of the plurality of human patients relative to a reference cerebral fibrillary amyloid burden in a predefined region of interest, wherein the reference cerebral fibrillary amyloid burden in a predefined region of interest is the cerebral fibrillary amyloid burden in a predefined region of interest of a plurality of human patients who have received the placebo. In some embodiments, administering such treatment according to the methods herein results in a reduction of annualized rate of change in amyloid burden in the plurality of human patients relative to a reference annualized rate of change in amyloid burden, wherein the reference annualized rate of change in amyloid burden is the annualized rate of change in amyloid burden (e.g., measured by PET) in a plurality of patients who received the placebo. In some embodiments, the cerebral fibrillary amyloid burden is measured using a fluorine labeled probe. In some embodiments, the fluorine label comprises fluorine-18 (18F). In some embodiments, the probe is florbetapir (see, e.g., Wong. J. Nucl. Med., 2010; 51 (6): 913-920). Florbetapir is an 18F labeled compound that binds beta-amyloid and has been shown to accumulate in regions associated with beta-amyloid deposits. In some embodiments, the cerebral fibrillary amyloid burden is measured by positron emission tomography (PET). In some embodiments, a florbetapir PET scan is considered positive if, based on a centralized visual read of the scan, it establishes the presence of moderate-to-frequent neuritic plaques. In some embodiments, administering such treatment results in a statistically significant reduced decline in regional cerebral fibrillary amyloid burden of the plurality of human patients relative or compared to the reference cerebral fibrillary amyloid burden about five years to about eight years after the start of treatment.

In some embodiments, administering such treatment results in a reduction of cerebral fibrillary burden in a predefined region of interest of the plurality of human patients relative to a reference cerebral fibrillary amyloid burden in a predefined region of interest, wherein the reference cerebral fibrillary amyloid burden in a predefined region of interest is a cerebral fibrillary amyloid burden in a predefined region of interest of a plurality of human patients who have received the placebo. In some embodiments, cerebral fibrillary burden is measured using florbetapir positron emission tomography (PET). In some embodiments, administering such treatment results in a reduction of annualized rate of change in amyloid burden measured by PET in the plurality of human patients by at least 3% relative to a reference amyloid burden measured by PET. In some embodiments, administering such treatment results in a reduction of annualized rate of change in amyloid burden measured by PET in the plurality of human patients by at least 10% relative to a reference amyloid burden measured by PET. In some embodiments, administering such treatment results in a reduction of annualized rate of change in amyloid burden measured by PET in the plurality of human patients by about 3% to about 10% relative to a reference amyloid burden measured by PET. In some embodiments, administering such treatment results in a reduction of annualized rate of change in amyloid burden measured by PET in the plurality of human patients by about 3%, about 4%, about 5%, about 6%, about 7%, about 8%, about 9%, or about 10% relative to a reference amyloid burden measured by PET. In some embodiments, administering such treatment results in a reduction of annualized rate of change in amyloid burden measured by PET in the plurality of human patients by about 3%, relative to a reference amyloid burden measured by PET.

In some embodiments, cerebral fibrillary amyloid burden is measured in a predefined region of interest in the patient receiving the anti-amyloid beta (Aβ) antibody and/or the placebo. In some embodiments, cerebral fibrillary amyloid burden is measured in the brain. In some embodiments, cerebral fibrillary amyloid burden is measured in a region of the brain containing amyloid deposits. In some embodiments, cerebral fibrillary amyloid burden is measured repeatedly in the same region of the brain to assess the impact of the anti-amyloid beta (Aβ) antibody treatment on cerebral fibrillary burden.

In some embodiments, administering such treatment according to the methods herein results in a reduced decline in regional cerebral metabolic rate of glucose (CMRgI) of the plurality of human patients relative to a reference decline in CMRgI, wherein the reference decline in CMRgI is the decline in CMRgI of the plurality of human patients who have received the placebo. In some embodiments, CMRgI is measured using a fluorine labeled probe. In some embodiments, the fluorine label comprises fluorine-18 (18F). In some embodiments, the probe is fluorodeoxyglucose (FDG). In some embodiments, the CMRgI is measured using FDG-positron emission tomography (PET). In some embodiments, administering such treatment results in a reduced FDG PET measurement in the plurality of human patients relative to a reference FDG PET measurement and the reference FDG PET measurement is a FDG PET measurement of the plurality of human patients who have received the placebo. In some embodiments, administering such treatment results in a statistically significant reduced decline in regional CMRgI of the plurality of human patients relative or compared to the reference decline in CMRgI about 12 weeks after the start of treatment. In some embodiments, administering such treatment results in a statistically significant reduced decline in regional CMRgI of the plurality of human patients relative or compared to the reference decline in CMRgI about five years to about eight years after the start of treatment.

In some embodiments, administering such treatment results in a reduced annualized FDG PET measurement in the plurality of human patients relative to a reference annualized FDG PET measurement. In some embodiments, administering such treatment results in a reduced annualized Standardized Uptake Value Ratio (SUVR) of FDG PET measurement in the plurality of human patients as compared to a reference annualized SUVR of FDG PET. In some embodiments, administering such treatment results in a reduced annualized Standardized Uptake Value Ratio (SUVR) of FDG PET measurement by at least 10% in the plurality of human patients as compared to a reference annualized SUVR of FDG PET. In some embodiments, administering such treatment results in a reduced annualized Standardized Uptake Value Ratio (SUVR) of FDG PET measurement by at least 20% in the plurality of human patients as compared to a reference annualized SUVR of FDG PET. In some embodiments, administering such treatment results in a reduced annualized Standardized Uptake Value Ratio (SUVR) of FDG PET measurement by about 10% to about 30% in the plurality of human patients as compared to a reference annualized SUVR of FDG PET. In some embodiments, administering such treatment results in a reduced annualized Standardized Uptake Value Ratio (SUVR) of FDG PET measurement by about 10%, about 11%, about 12%, about 13%, about 14%, about 15%, about 16%, about 17%, about 18%, about 19%, or about 20% in the plurality of human patients as compared to a reference annualized SUVR of FDG PET. In some embodiments, administering such treatment results in a reduced annualized Standardized Uptake Value Ratio (SUVR) of FDG PET measurement by about 18% in the plurality of human patients as compared to a reference annualized SUVR of FDG PET.

In some embodiments, administering such treatment according to the methods herein results in a reduced brain atrophy of the plurality of human patients relative to a reference brain atrophy, wherein the reference brain atrophy is the brain atrophy of the plurality of human patients who have received the placebo. In some embodiments, administering such treatment results in a statistically significant reduced brain atrophy of the plurality of human patients relative or compared to the reference brain atrophy about 12 weeks after the start of treatment. In some embodiments, administering such treatment results in a statistically significant reduced brain atrophy of the plurality of human patients relative or compared to the reference brain atrophy about five years to about eight years after the start of treatment. In some embodiments, the brain atrophy is measured using volumetric MRI. In some embodiments, the volumetric MRI is measured in a whole brain. In some embodiments, administering such treatment results in a reduction of an annualized rate of change in the brain atrophy of the plurality of human patients by at least 60% relative to the reference brain atrophy, said reduction being measured by a volumetric MRI on a whole brain. In some embodiments, administering such treatment results in a reduction of an annualized rate of change in the brain atrophy of the plurality of human patients by at least 70% relative to the reference brain atrophy, said reduction being measured by a volumetric MRI on a whole brain. In some embodiments, administering such treatment results in a reduction of an annualized rate of change in the brain atrophy of the plurality of human patients by about 60% to about 70% relative to the reference brain atrophy, said reduction being measured by a volumetric MRI on a whole brain. In some embodiments, administering such treatment results in a reduction of an annualized rate of change in the brain atrophy of the plurality of human patients by about 60%, about 61%, about 62%, about 63%, about 64%, about 65%, about 66%, about 67%, about 68%, about 69%, or about 70% relative to the reference brain atrophy, said reduction being measured by a volumetric MRI on a whole brain.

In some embodiments, the volumetric MRI is measured in a bilateral hippocampus. In some embodiments, administering such treatment results in a reduction of an annualized rate of change in the brain atrophy of the plurality of human patients by at least 5% relative to the reference brain atrophy, said reduction being measured by a volumetric MRI on a bilateral hippocampus. In some embodiments, administering such treatment results in a reduction of an annualized rate of change in the brain atrophy of the plurality of human patients by at least 10% relative to the reference brain atrophy, said reduction being measured by a volumetric MRI on a bilateral hippocampus. In some embodiments, administering such treatment results in a reduction of an annualized rate of change in the brain atrophy of the plurality of human patients by about 5% to about 20% relative to the reference brain atrophy, said reduction being measured by a volumetric MRI on a bilateral hippocampus. In some embodiments, administering such treatment results in a reduction of an annualized rate of change in the brain atrophy of the plurality of human patients by about 5%, about 6%, about 7%, about 8%, about 9%, or about 10% relative to the reference brain atrophy, said reduction being measured by a volumetric MRI on a bilateral hippocampus.

In some embodiments, administering such treatment according to the methods herein results in a reduction of an annualized rate of change in the brain atrophy of the plurality of human patients relative to a reference brain atrophy, wherein the reference brain atrophy is the annualized rate of change in brain atrophy of the plurality of human patients who have received the placebo. In some embodiments, administering such treatment results in a statistically significant reduced brain atrophy of the plurality of human patients relative or compared to the reference brain atrophy about 12 weeks after the start of treatment. In some embodiments, administering such treatment results in a statistically significant reduced brain atrophy of the plurality of human patients relative or compared to the reference brain atrophy about five years to about eight years after the start of treatment. In some embodiments, the brain atrophy is measured using volumetric MRI. In some embodiments, the volumetric MRI is measured in a whole brain. In some embodiments, a whole brain shrinkage is measured using a volumetric MRI. In some embodiments, administering such treatment results in a reduction of annualized rate of change in the plurality of human patients by at least 5% relative to the reference brain atrophy, said reduction being measured by a volumetric MRI on a whole brain. In some embodiments, administering such treatment results in a reduction of an annualized rate of change in the brain atrophy of the plurality of human patients by at least 10% relative to the reference brain atrophy, said reduction being measured by a volumetric MRI on a whole brain. In some embodiments, administering such treatment results in a reduction of an annualized rate of change in the brain atrophy of the plurality of human patients by about 5% to about 20% relative to the reference brain atrophy, said reduction being measured by a volumetric MRI on a whole brain. In some embodiments, administering such treatment results in a reduction of an annualized rate of change in in the brain atrophy of the plurality of human patients by about 5%, about 6%, about 7%, about 8%, about 9%, about 10%, about 11%, about 12%, about 13%, about 14%, about 15%, about 16%, about 17%, about 18%, about 19%, about 20%, about 25%, or about 30% relative to the reference brain atrophy, said reduction being measured by a volumetric MRI on a whole brain.

In some embodiments, the volumetric MRI is measured in a bilateral hippocampus. In some embodiments, a hippocampal shrinkage is measured using a volumetric MRI. In some embodiments, administering such treatment results in a reduction of an annualized rate of change in in the brain atrophy of the plurality of human patients by at least 1% relative to the reference brain atrophy, said reduction being measured by a volumetric MRI on a bilateral hippocampus. In some embodiments, administering such treatment results in a reduction of an annualized rate of change in the brain atrophy of the plurality of human patients by at least 2% relative to the reference brain atrophy, said reduction being measured by a volumetric MRI on a bilateral hippocampus. In some embodiments, administering such treatment results in a reduction of an annualized rate of change in the brain atrophy of the plurality of human patients by about 1% to about 10% relative to the reference brain atrophy, said reduction being measured by a volumetric MRI on a bilateral hippocampus. In some embodiments, administering such treatment results in a reduction of an annualized rate of change in the brain atrophy of the plurality of human patients by about 1%, about 2%, about 3%, about 4%, about 5%, or about 10% relative to the reference brain atrophy, said reduction being measured by a volumetric MRI on a bilateral hippocampus.

In some embodiments, administering such treatment according to the methods herein results in a reduced annualized rate of change in the brain atrophy of the plurality of human patients relative to a reference annualized rate of change in the brain atrophy, wherein the reference annualized rate of change in the brain atrophy is the annualized rate of change in the brain atrophy of the plurality of human patients who have received the placebo. In some embodiments, administering such treatment results in a statistically significant reduced annualized rate of change in the brain atrophy of the plurality of human patients relative or compared to the reference annualized rate of change in the brain atrophy about 12 weeks after the start of treatment. In some embodiments, administering such treatment results in a statistically significant reduced annualized rate of change in the brain atrophy of the plurality of human patients relative or compared to the reference annualized rate of change in the brain atrophy about five years to about eight years after the start of treatment. In some embodiments, the brain atrophy is measured using volumetric MRI.

In some embodiments, administering such treatment results in a reduction of cerebrospinal fluid (CSF) neurofilament light (CSF NfL) in the plurality of human patients as compared to a reference CSF NIL, wherein the reference CSF NfL is from the plurality of human patients who have received the placebo. In some embodiments, administering such treatment results in a relative reduction of CSF NfL in the plurality of human patients of at least 10% relative to the reference CSF NfL. In some embodiments, administering such treatment results in a relative reduction of CSF NfL in the plurality of human patients of at least 20% relative to the reference CSF NfL. In some embodiments, administering such treatment results in a relative reduction of CSF NfL in the plurality of human patients of about 10% to about 30% relative to the reference CSF NfL. In some embodiments, administering such treatment results in a relative reduction of CSF NfL in the plurality of human patients of about 10%, about 11%, about 12%, about 13%, about 14%, about 15%, about 16%, about 17%, about 18%, about 19%, about 20%, about 35%, or about 30% relative to the reference CSF NfL. In some embodiments, administering such treatment results in a relative reduction of CSF NfL in the plurality of human patients of about 18% relative to the reference CSF NfL.

In some embodiments, administering such treatment according to the methods herein results in results in an effect on a plasma biomarker of the plurality of human patients compared to a reference plasma biomarker, wherein the reference plasma biomarker is the plasma biomarker of a plurality of human patients who have received the placebo. In some embodiments, the plasma biomarker is measured using an immunoassay. In some embodiments, the plasma biomarker is any one of Aβ42, Aβ40, pTau181, pTau217, NIL, GFAP, YKL-40, or sTREM. In some embodiments, the plasma biomarker is the ratio of Aβ42 to Aβ40.

In some embodiments, administering such treatment results in an increase in of annualized rate of change in the plasma Aβ42 biomarker in the plurality of human patients compared to a reference annualized rate of change in the plasma Aβ42 biomarker, wherein the reference plasma Aβ42 biomarker is an annualized rate of change in the plasma Aβ42 biomarker of a plurality of human patients who have received the placebo.

In some embodiments, administering such treatment results in an increase in of annualized rate of change in the plasma Aβ40 biomarker in the plurality of human patients compared to a reference annualized rate of change in the plasma Aβ40 biomarker, wherein the reference plasma Aβ40 biomarker is an annualized rate of change in the plasma Aβ40 biomarker of a plurality of human patients who have received the placebo.

In some embodiments, administering such treatment results in a reduction of annualized rate of change in the plasma pTau181 biomarker in the plurality of human patients compared to a reference annualized rate of change in the plasma pTau181 biomarker, wherein the reference plasma pTau181 biomarker is an annualized rate of change in the plasma pTau181 biomarker of a plurality of human patients who have received the placebo. In some embodiments, administering such treatment results in a relative reduction of pTau181 in the plurality of human patients of at least about 2% relative to the reference pTau181. In some embodiments, administering such treatment results in a relative reduction of pTau181 in the plurality of human patients of at least about 5% relative to the reference pTau181. In some embodiments, administering such treatment results in a relative reduction of pTau181 in the plurality of human patients of about 2% to about 10% relative to the reference pTau181. In some embodiments, administering such treatment results in a relative reduction of pTau181 in the plurality of human patients of about 2%, about 3%, about 4%, about 5%, about 6%, about 7%, about 8%, about 9%, or about 10% relative to the reference pTau181. In some embodiments, administering such treatment results in a relative reduction of pTau181 in the plurality of human patients of about 6% or about 6.2% relative to the reference pTau181.

In some embodiments, administering such treatment results in a reduction of annualized rate of change in the plasma pTau217 biomarker in the plurality of human patients compared to a reference annualized rate of change in the plasma pTau217 biomarker, wherein the reference plasma pTau217 biomarker is an annualized rate of change in the plasma pTau217 biomarker of a plurality of human patients who have received the placebo. In some embodiments, administering such treatment results in a relative reduction of pTau217 in the plurality of human patients of at least about 5% relative to the reference pTau217. In some embodiments, administering such treatment results in a relative reduction of pTau217 in the plurality of human patients of at least about 10% relative to the reference pTau217. In some embodiments, administering such treatment results in a relative reduction of pTau217 in the plurality of human patients of about 5% to about 15% relative to the reference pTau217. In some embodiments, administering such treatment results in a relative reduction of pTau217 in the plurality of human patients of about 5%, about 6%, about 7%, about 8%, about 9%, or about 10%, about 11%, about 12%, about 13%, about 14%, or about 15% relative to the reference pTau217. In some embodiments, administering such treatment results in a relative reduction of pTau217 in the plurality of human patients of about 9% relative to the reference pTau217.

In some embodiments, administering such treatment results in a reduction of annualized rate of change in the plasma NfL biomarker in the plurality of human patients compared to a reference annualized rate of change in the plasma NfL biomarker, wherein the reference plasma NfL biomarker is an annualized rate of change in the plasma NfL biomarker of a plurality of human patients who have received the placebo. In some embodiments, administering such treatment results in a relative reduction of NfL in the plurality of human patients of at least about 5% relative to the reference NfL. In some embodiments, administering such treatment results in a relative reduction of NfL in the plurality of human patients of at least about 10% relative to the reference NfL. In some embodiments, administering such treatment results in a relative reduction of NfL in the plurality of human patients of about 5% to about 15% relative to the reference NfL. In some embodiments, administering such treatment results in a relative reduction of NfL in the plurality of human patients of about 5%, about 6%, about 7%, about 8%, about 9%, about 10%, about 11%, about 12%, about 13%, about 14%, or about 15% relative to the reference NfL. In some embodiments, administering such treatment results in a relative reduction of NfL in the plurality of human patients of about 10%, or about 10.5% relative to the reference NfL.

In some embodiments, administering such treatment results in a reduction of annualized rate of change in the plasma GFAP biomarker in the plurality of human patients compared to a reference annualized rate of change in the plasma GFAP biomarker, wherein the reference plasma GFAP biomarker is an annualized rate of change in the plasma GFAP biomarker of a plurality of human patients who have received the placebo. In some embodiments, administering such treatment results in a relative reduction of GFAP in the plurality of human patients of at least about 10% relative to the reference GFAP. In some embodiments, administering such treatment results in a relative reduction of GFAP in the plurality of human patients of at least about 15% relative to the reference GFAP. In some embodiments, administering such treatment results in a relative reduction of GFAP in the plurality of human patients of about 10% to about 20% relative to the reference GFAP. In some embodiments, administering such treatment results in a relative reduction of GFAP in the plurality of human patients of about 10%, about 11%, about 12%, about 13%, about 14%, about 15%, about 16%, about 17%, about 18%, about 19%, or about 20% relative to the reference GFAP. In some embodiments, administering such treatment results in a relative reduction of GFAP in the plurality of human patients of about 17% or about 17.7% relative to the reference GFAP.

In some embodiments, administering such treatment results in a reduction of annualized rate of change in the plasma YKL-40 biomarker in the plurality of human patients compared to a reference annualized rate of change in the plasma YKL-40 biomarker, wherein the reference plasma YKL-40 biomarker is an annualized rate of change in the plasma YKL-40 biomarker of a plurality of human patients who have received the placebo. In some embodiments, administering such treatment results in a relative reduction of YKL-40 in the plurality of human patients of at least about 8% relative to the reference YKL-40. In some embodiments, administering such treatment results in a relative reduction of YKL-40 in the plurality of human patients of at least about 15% relative to the reference YKL-40. In some embodiments, administering such treatment results in a relative reduction of YKL-40 in the plurality of human patients of about 8% to about 20% relative to the reference YKL-40. In some embodiments, administering such treatment results in a relative reduction of YKL-40 in the plurality of human patients of about 8%, about 9%, about 10%, about 11%, about 12%, about 13%, about 14%, about 15%, about 16%, about 17%, about 18%, about 19%, or about 20% relative to the reference YKL-40. In some embodiments, administering such treatment results in a relative reduction of YKL-40 in the plurality of human patients of about 12% or about 12.4% relative to the reference YKL-40.

In some embodiments, administering such treatment results in a reduction of annualized rate of change in the plasma sTREM2 biomarker in the plurality of human patients compared to a reference annualized rate of change in the plasma sTREM2 biomarker, wherein the reference plasma sTREM2 biomarker is an annualized rate of change in the plasma sTREM2 biomarker of a plurality of human patients who have received the placebo. In some embodiments, administering such treatment results in a relative reduction of sTREM2 in the plurality of human patients of at least about 15% relative to the reference sTREM2. In some embodiments, administering such treatment results in a relative reduction of sTREM2 in the plurality of human patients of at least about 25% relative to the reference sTREM2. In some embodiments, administering such treatment results in a relative reduction of sTREM2 in the plurality of human patients of about 15% to about 30% relative to the reference sTREM2. In some embodiments, administering such treatment results in a relative reduction of sTREM2 in the plurality of human patients of about 15%, about 16%, about 17%, about 18%, about 19%, about 20%, about 21%, about 22%, about 23%, about 24%, about 25%, about 26%, about 27%, about 28%, about 29%, or about 30% relative to the reference sTREM2. In some embodiments, administering such treatment results in a relative reduction of sTREM2 in the plurality of human patients of about 23% or about 23.1 relative to the reference sTREM2.

### III. Patient Populations

The present invention provides methods for delaying onset of at least one symptom or slowing cognitive decline in a human patient with a genetic mutation that causes familial Alzheimer's Disease (AD). In some embodiments, the mutation is an autosomal mutation. In some embodiments, the mutation is an autosomal dominant mutation.

In some embodiments, the genetic mutation that causes familial AD is a mutation in any one or more of presenilin 1 (PSEN1), presenilin 2 (PSEN2), and/or amyloid precursor protein (APP). In some embodiments, the mutation is an autosomal dominant mutation. Autosomal dominant mutations in PSEN1, PSEN2, and APP are known in the art (see, e.g., the mutation database maintained on the world wide web by ALZFORUM, which is herein incorporated by reference in its entirety).

In some embodiments, the mutation that causes familial AD is a mutation in PSEN1. In some embodiments, the mutation comprises one or more of E280A, P117L, M139T, M146L, M146V. H163R, L166P. M233T, L235P, A246E, P264L, R2781, L286V, and L435F. In some embodiments, the genetic mutation is an E280A mutation in PSEN1.

In some embodiments, the mutation that causes familial AD is a mutation in PSEN2. In some embodiments, the mutation comprises one or more of N1411, T122P, M239V, M2391, and M298T.

In some embodiments, the mutation that causes familial AD is a mutation in APP. In some embodiments, the mutation comprises one or more of KM670/671NL, A673T, E693Q. E693G, E693del, D694N, T7141, 1716F, 1716V, V717F, and V7171.

In some embodiments, the human patient is 100 years of age or younger. In some embodiments, the human patient is 60 years of age or younger. In some embodiments, the human patient is 20 years of age or older. In some embodiments, the human patient is 30 years of age or older. In some embodiments, the human patient is between 20 years and 100 years in age. In some embodiments, the human patient is between 20 years and 30 years in age. In some embodiments, the human patient is between 20 years and 40 years in age. In some embodiments, the human patient is between 20 years and 50 years in age. In some embodiments, the human patient is between 20 years and 60 years in age. In some embodiments, the human patient is between 20 years and 70 years in age. In some embodiments, the human patient is between 20 years and 80 years in age. In some embodiments, the human patient is between 20 years and 90 years in age. In some embodiments, the human patient is between 30 years and 40 years in age. In some embodiments, the human patient is between 30 years and 50 years in age. In some embodiments, the human patient is between 30 years and 60 years in age. In some embodiments, the human patient is between 30 years and 70 years in age. In some embodiments, the human patient is between 30 years and 80 years in age. In some embodiments, the human patient is between 30 years and 90 years in age. In some embodiments, the human patient is between 30 years and 100 years in age. In some embodiments, the human patient is between 40 years and 50 years in age. In some embodiments, the human patient is between 40 years and 60 years in age. In some embodiments, the human patient is between 40 years and 70 years in age. In some embodiments, the human patient is between 40 years and 80 years in age. In some embodiments, the human patient is between 40 years and 90 years in age. In some embodiments, the human patient is between 40 years and 100 years in age. In some embodiments, the human patient is between 50 years and 60 years in age. In some embodiments, the human patient is between 50 years and 70 years in age. In some embodiments, the human patient is between 50 years and 80 years in age. In some embodiments, the human patient is between 50 years and 90 years in age. In some embodiments, the human patient is between 50 years and 100 years in age. In some embodiments, the human patient is between 60 years and 70 years in age. In some embodiments, the human patient is between 60 years and 80 years in age. In some embodiments, the human patient is between 60 years and 90 years in age. In some embodiments, the human patient is between 60 years and 100 years in age. In some embodiments, the human patient is between 70 years and 80 years in age. In some embodiments, the human patient is between 70 years and 90 years in age. In some embodiments, the human patient is between 70 years and 100 years in age. In some embodiments, the human patient is between 80 years and 90 years in age. In some embodiments, the human patient is between 80 years and 100 years in age. In some embodiments, the human patient is between 90 years and 100 years in age. In some embodiments, the human patient is between 30 years and 60 years in age.

In some embodiments, the human patient is characterized as having preclinical AD, e.g., the patient is at certain risk of developing AD dementia but does not yet have over symptoms and does not meet criteria for MCI or dementia (see, e.g., Reiman et al., Biomark. Med., 2010, 4:3-14, Sperling et al., Alzheimers Dement, 2011, 7:280-292).

In some embodiments, the human patient does not meet the criteria for dementia due to AD as defined by Mckhann et al., Alzheimers Dement., 2011; 7:263-269.

In some embodiments, the patient has an MMSE of ≥24 for patients with less than 9 years of education or an MMSE of ≥26 for patients with 9 or more years of education.

In some embodiments, the human patient does not meet the criteria for mild cognitive impairment (MCI) due to AD (see, e.g., Albert et al., Alzheimers Dement., 2011; 7:270-279). In some embodiments, the criteria for MCI due to AD comprise: Cognitive concern in the judgment of the physician, based in part on the average Subjective Memory Checklist score >22 (average of participant and study partner component scores); Word List: Recall <3 for participants with less than 9 years of education, or Word List: Recall <5 for participants with 9 or more years of education; Preservation of independence in functional activities in the judgment of the physician, based in part on review of the Functional assessment staging (FAST) (see, e.g., Sclan and Reisberg, Int. Psychogeriatr, 1992; 4:55-69.).

In some embodiments, the human patient has adequate vision and hearing in the physician's judgment to be able to complete one or more cognitive, functional, and/or behavioral assessments.

In some embodiments, the human patient is paired with a study partner who participates in the treatment, but does not receive the treatment. In some embodiments, the study partner accompanies the patient to all required clinical visits and/or treatment related events. In some embodiments, the study partner provides telephonic assessments of the patient. In some embodiments, the study partner spends sufficient time with the patient to be familiar with the overall function and behavior of the patient and is able to provide adequate information regarding the patient, including information about the patient's domestic activities, hobbies, routines, social skills, basic activities of daily life, work and educational history; the patient's cognitive performance, including memory abilities, language abilities, temporal and spatial orientation, judgment, and problem solving; the patient's emotional and psychological state; and the patient's general health status. In some embodiments, the patient and study partner provide evidence of adequate premorbid functioning (e.g., intellectual, visual, and auditory). In some embodiments, the patient and study partner have evidence of fluency in and ability to read the language in which study assessments are administered.

In some embodiments, the human patient is willing and able to undergo neuroimaging. In some embodiments, the neuroimaging may include, but is not limited to, positron emission tomography (PET). In some embodiments, the neuroimaging may include, but is not limited to, magnetic resonance imaging (MRI).

In some embodiments, the human patient is in good general health. In some embodiments, the human patient has no known co-morbidities expected to interfere with the methods described herein.

In some embodiments, the human patient does not display body weight <45 kg. In some embodiments, the patient does not display body weight >120 kg.

In some embodiments, the human patient does not display brain MRI imaging results at baseline showing evidence of any of the following: (a) any ARIA-E (cerebral VE, sulcal effusion), infection, significant cerebral vascular pathology, clinically significant lacunar infarct in a region important for cognition or multiple lacunes or a cortical infarct or focal lesions of clinical significance; (b) more than four cerebral microhemorrhages (lesions with diameter ≤10 mm), regardless of their anatomical location or diagnostic characterization as “possible” or “definite”; and (c) single area of superficial siderosis of the CNS or evidence of a prior cerebral macrohemorrhage (lesion with diameter >0 mm).

In some embodiments, the human patient does not use any other medications that have the potential to significantly affect cognition. In some embodiments, other medications include, but are not limited to, sedatives, narcotics (e.g., opiates/opioids), hypnotics, over-the-counter (OTC) sleeping aids, or sedating anti-allergy medications. In some embodiments, the patient may use these medications short-term or intermittently as deemed medically necessary for the treatment of a non-excluded medical condition. In some embodiments, patients may use stable low doses of tricyclic antidepressants or benzodiazepines for the treatment of a non-excluded medical condition.

In some embodiments, the human patient does not use typical anti-psychotics or barbiturates. In some embodiments, the patient does not use non-anti-cholinergic antidepressant medications or atypical anti-psychotics unless maintained on a stable dose regimen for at least 6 weeks prior to treatment. In some embodiments, the patient does not use any FDA/INVIMA-approved medications for treatment of late-onset AD at baseline. In some embodiments, the patient does not use anti-coagulant medication. In some embodiments, the patient does not display a known coagulopathy or platelet count <100,000 cells/μL within 4 weeks of the treatment visit. In some embodiments, the patient does not use any biologic therapy within five half-lives or 3 months prior to treatment, whichever is longer. In some embodiments, the patient may use routinely recommended vaccinations. In some embodiments, the patient does not use anti-seizure, anti-Parkinsonian, or stimulant (e.g., methylphenidate) medications. In some embodiments, the patient does not use any investigational drug, device, or experimental medication within 60 days (or five half-lives, whichever is longer) of the treatment visit. In some embodiments, the patient does not have a history of previous treatment with crenezumab (MABT5102A) or any other therapeutic that targets Aβ. In some embodiments, the patient does not have a history of severe allergic, anaphylactic, or other hypersensitivity reactions to chimeric, human, or humanized antibodies or fusion proteins.

In some embodiments, the methods comprise comparison to a reference, e.g. a reference onset of at least one symptom or a reference level of a biomarker. In some embodiments, the reference is established by a plurality of human patients receiving a placebo. In some embodiments, the plurality of human patients receiving the placebo comprise carriers of a familial AD genetic mutation. In some embodiments, the plurality of human patients receiving the placebo comprise non-carriers of a familial AD genetic mutation. In some embodiments, the plurality of human patients receiving the placebo comprise both carriers of a familial AD genetic mutation and non-carriers of a familial AD genetic mutation.

### IV. Kits and Articles of Manufacture

In another aspect of the invention, an article of manufacture or a kit containing materials useful for the treatment of a patient having preclinical ADAD. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating preclinical ADAD and may have a sterile access port (for example the container may be an intravenous solution bag or a vial having a stopper pierceable by a hypodermic injection needle). At least one active agent in the composition is an anti-Aβ antibody. The label or package insert indicates that the composition is used for treating preclinical ADAD. In some embodiments, the article of manufacture or kit further comprises a package insert comprising instructions for using the anti-Aβ antibody to treat ADAD. The article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

### Example 1—Clinical Study of the Safety and Efficacy of Crenezumab, a Humanized Anti-Aβ Monoclonal Antibody, Administered to PSEN1 E280A Mutation Carrier and Non-Carrier Patients to Prevent Onset of Autosomal-Dominant Alzheimer's Disease

Autosomal-dominant Alzheimer's Disease (ADAD; also known as familial AD or dominantly-inherited AD (DIAD)) is a rare, inherited form of AD caused by single gene mutations in the APP, PSEN1, or PSEN2 genes. Less than 1% of all AD cases worldwide are thought to be caused by genetic mutations. ADAD displays a much earlier onset than the more common sporadic AD, with symptoms typically developing between 30-60 years of age. An individual carrying one of these mutations is nearly certain to develop AD and has a 50% risk of allele heritability. One particular mutation, the PSEN1 E280A or ‘Paisa’ mutation, originates from a kindred in the Antioquia region of Colombia. Within this kindred 6,000 members are impacted and over 1.200 members are confirmed carriers. Mutation carriers develop memory deficits in their 30s; full dementia develops between 45-50 years of agc.

Crenczumab (RO5490245; MABT5102A), is a fully humanized IgG4 monoclonal antibody to Ab selected for its ability to bind monomeric, oligomeric, and fibrillar forms of Aβ in vitro (Adolfsson et al., J Neurosci, 2012; 32:9677-9689). Crenczumab binds both Aβ1-40 and Aβ1-42, inhibits Aβ aggregation, and promotes Aβ disaggregation. Because crenezumab is a human IgG4 backbone antibody, it has reduced Fcγ receptor (FcγR)-binding affinity compared with human IgG1 or IgG2, which is predictive of reduced immune-effector response. These properties, combined with the ability of systemically delivered crenezumab to decrease Aβ CNS levels in a murine model of AD, suggest that this anti-Aβ therapeutic approach may offer clinical efficacy while reducing risk of toxicity, with the ultimate goal of modifying disease progression in patients with AD with lower risk of amyloid-related imaging abnormalities-edema/effusion (ARIA-E), including cerebral vasogenic edema (VE) and sulcal effusion, or amyloid-related imaging abnormalities-hemosiderin deposition (ARIA-H), including cerebral microhemorrhages and superficial siderosis (Sperling et al., Alzheimers Dement., 2011; 7:367-385). The encouraging safety profile to date offers the opportunity to administer high doses of the agent and achieve high brain concentrations relative to alternative monoclonal antibodies. Indeed, this safety profile allows doses higher than 720 mg SC every 2 weeks (Q2W) to be studied in participants. Specifically, the dose of 60 mg/kg IV every 4 weeks (Q4W) was included as an option for participants in this study (GN28352, amendment 6). This dose was also evaluated in a Phase Ib study (GN29632), in the global Phase III studies (BN29552 and BN29553) in sporadic prodromal to mild AD and the open-label extension (OLE) study BN40031.

The API ADAD trial (NCT01998841) was a prospective, randomized, double-blind, placebo-controlled, parallel-group enabling Phase II study of the efficacy of crenezumab versus placebo in cognitively healthy individuals who had no clinical symptoms of Alzheimer's Disease but carried the PSEN1 E280A autosomal dominant mutation.

### Study Design and Objectives

This clinical study was comprised of two study periods, termed Study Periods A and B. Study Period A was a prospective, randomized, double-blind, placebo-controlled, parallel-group study of crenezumab versus placebo treatment in carriers of PSEN1 E280A in autosomal-dominant, PSEN1-mutation-causing early-onset AD who did not meet criteria for mild cognitive impairment (MCI) due to AD or dementia due to AD and were, thus, in a preclinical phase of AD. The study also incorporated administration of placebo to individuals who were not PSEN1 E280A autosomal-dominant mutation carriers.

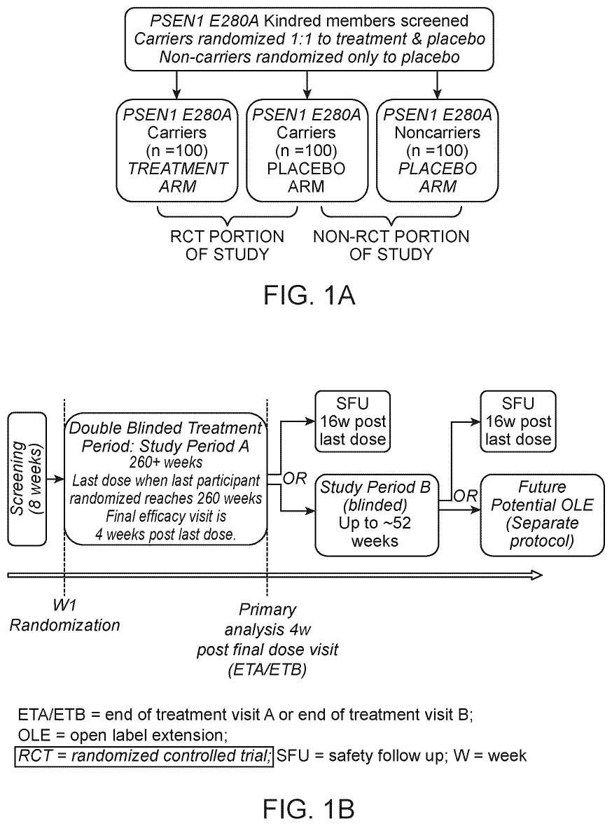

PSEN1 E280A autosomal-dominant mutation carriers who met study eligibility criteria were randomized in a 1:1 ratio to one of two treatment groups: crenezumab or placebo. The study was originally powered to compare the mean change from baseline over 260 weeks in the API ADAD Composite Cognitive Test total score between the active group and the placebo. Assuming a 25% dropout rate, two-sided testing at an overall 0.05 level, a placebo group CV of 65% for the week 260 change scores (=100%×standard deviation of placebo participant change scores/mean of placebo participant change scores) and 100 participants per arm, the study would have at least 80% power to detect a true effect of 30% reduction of the mean decline in the placebo group. In order to maintain genotype blinding and to have a genetic kindred control, a cohort of PSEN1 E280A mutation non-carrier kindred family members were also enrolled in the study and dosed only with placebo. Thus, the study included three arms with approximately 100 participants per arm; two arms of participants with a PSEN1 E280A mutation randomized in a 1:1 ratio to active or placebo and a third arm of non-carriers randomized to placebo (see FIG. 1A).

Participants received the randomized treatment until the final participant had reached their final treatment visit at 260 weeks. The study duration for individual participants was at least approximately 284 weeks, including an 8-week screening period, a double-blind treatment period of at least 260 weeks in length, a 4-week final dose visit, and a final safety follow-up visit 16 weeks after the last dose of study drug (crenezumab or placebo) that allowed for clinical follow-up after treatment discontinuation for participants who did not to continue with study drug beyond the end of Study Period A or who terminated study drug early (see FIG. 1B).

Crenezumab was administered either subcutaneously (720 mg every 2 weeks) or intravenously (60 mg/kg every 4 weeks), and matching placebo was administered by the same routes at the same frequencies. The switch to the higher intravenous dose (approximately 4-fold higher exposure to crenczumab) was optional. Participants could decide whether to change from the initial subcutaneous dosing to the IV dosing; however, once intravenous dosing in a given participant had occurred, participants could not switch back to the lower subcutaneous dose.

Following the completion of Study Period A, participants were offered the opportunity to continue in Study Period B. This second study period involved participants continuing to receive study drug until the results of the study were known and post-trial access to crenezumab was started via an open label extension (OLE) or other program, or development of crenezumab was discontinued. All PSEN1 E280A mutation carriers were provided crenezumab in Study Period B regardless of treatment allocation during Study Period A, and all non-carriers continued on placebo. Treatment allocation for both study periods A and B remained blinded. Study Period B lasted up to approximately 9-12 months based on when participants entered Study Period B.

The target population included individuals who were members of a PSEN1 E280A mutation carrier kindred and agreed to conditions of, and were willing to undergo, genetic testing (e.g., APOE, PSEN1 E280A, and other genetic testing) to have PSEN1 E280A mutation carrier or non-carrier status confirmed prior to or during the screening period. Patient demographic data is included below in Table 1. Patients were males and females of the age range ≥30 years and ≤60 years; had MMSE of ≥24 for participants with less than 9 years of education or MMSE of ≥26 for participants with 9 or more years of education; did not meet criteria for dementia due to AD; did not meet criteria for MCI due to AD as defined by the following: (a) cognitive concern based in part on the average Subjective Memory Checklist score >22 (average of participant and study partner component scores), (b) Word List: Recall <3 for participants with less than 9 years of education, (c) Word List: Recall <5 for participants with 9 or more years of education, and (d) preservation of independence in functional activities based in part on review of the Functional assessment staging (FAST); demonstrated adequate vision and hearing to be able to complete testing; had a study partner who agreed to participate in the study and was capable of and willing to: (a) accompany the participant to all required visits for subcutaneous administration or intravenous administration, (b) provide information for required telephone assessments, (c) spend sufficient time with the participant to be familiar with his/her overall function and behavior and be able to provide adequate information about the participant including: (i) knowledge about domestic activities, hobbies, routines, social skills and basic activities of daily life, (ii) work and educational history, (iii) cognitive performance, including memory abilities, language abilities, temporal and spatial orientation, judgment, and problem solving, (iv) emotional and psychological state, and (v) general health status. The participant and study partner had evidence of the following: (a) adequate premorbid functioning (e.g., intellectual, visual, and auditory), and (b) fluency in, and able to read, the language in which study assessments were administered. Participants were willing and able to undergo neuroimaging (PET and MRI); displayed no clinically significant thyroid dysfunction or B12 deficiency as determined by the following criteria: (a) serum thyroid stimulating hormone (TSH) and B12 levels within normal or expected ranges for the testing laboratory, (b) if the participant was undergoing thyroid replacement therapy, TSH levels were either within normal or expected ranges for the testing laboratory, or (c) if the participant was receiving vitamin B12 injections or oral vitamin B12 therapy, B12 levels were at or above the lower limit of normal for the testing laboratory; and were in good general health with no known co-morbidities expected to interfere with participation in the study.

TABLE 1
Participant selected baseline demographics and clinical characteristics*.

| | Carriers | Non-Carriers | P-value |
|---|---|---|---|
| N | 167 | 75 | — |
| Age (range) | 37 ± 5 (30-53) | 42 ± 6 (30-53) | <0.001 |
| Sex (n [%] female) | 101 (60%) | 50 (67%) | 0.36 |
| Education (years) | 8.8 ± 4.1 | 8.5 ± 4.4 | 0.64 |
| APOE4 carriers (n [%]) | 36 (22%) | 17 (29%) | 0.85 |
| ARIA-E | 0% | 0% | — |
| 1-3 microhemorrhages (n | 5 (3%) | 6 (8%) | 0.19 |
| [%]) | | | |

*Data from 242 age range-matched mutation carriers & non-carriers that are available prior to trial completion. (Tariot et al., (2018) CTAD)

The target population excluded anyone with (1) significant medical, psychiatric, or neurological condition or disorder documented by history, physical, neurological, laboratory, or ECG examination that would place the participant at undue risk or impact the interpretation of efficacy; (2) history of stroke; (3) history of severe, clinically significant CNS trauma (e.g., cerebral contusion); (4) body weight <45 or >120 kg; (5) history or presence of atrial fibrillation that posed a risk for future stroke; (6) clinically significant laboratory or ECG abnormalities; (7) presence of bipolar disorder or other clinically significant major psychiatric disorder according to DSM-IV-TR (recorded as an AE if diagnosed after study enrollment); (8) clinically significant depression, based in part by a Geriatric Depression Scale (short form GDS) (15-point scale) score >9 at screening; (9) history of seizures; (10) myocardial infarction within 2 years, congestive heart failure, atrial fibrillation, or uncontrolled hypertension; (11) history of cancer within 5 years; (12) clinically significant infection within the last 30 days prior to screening; (13) brain MRI imaging results at baseline showing any of the following: (a) evidence of any ARIA-E (cerebral VE, sulcal effusion), infection, significant cerebral vascular pathology, clinically significant lacunar infarct in a region important for cognition or multiple lacunes or a cortical infarct or focal lesions of clinical significance, (b) more than four cerebral microhemorrhages (lesions with diameter ≤10 mm), regardless of their anatomical location or diagnostic characterization as “possible” or “definite”, or (c) single area of superficial siderosis of the CNS or evidence of a prior cerebral macrohemorrhage (lesion with diameter >0 mm); (14) clinically significant screening blood or urine laboratory abnormalities requiring further evaluation or treatment, including: (a) impaired hepatic function, as indicated by transaminases >2× the upper limit of normal (ULN) or clinically significant abnormalities in synthetic function tests, (b) impaired coagulation (aPTT >1.2 × ULN), (c) platelet count <100,000/μL, or (d) glycosylated hemoglobin >8.0%; (15) positive urine test for drugs of abuse at screening (results of cannabinoid assays were not used for the determination of eligibility); (16) history of alcohol or substance dependence within the previous two years (DSM-IV TR criteria); (17) use of any other medications with the potential to significantly affect cognition (including, but not limited to, sedatives, narcotics (e.g., opiates/opioids), hypnotics, over-the-counter (OTC) sleeping aids, sedating anti-allergy medications); (18) use of typical anti-psychotics or barbiturates; (19) use of non-anti-cholinergic antidepressant medications or atypical anti-psychotics unless maintained on a stable dose regimen for at least 6 weeks prior to screening; (20) use of any FDA/INVIMA-approved medications for treatment of late-onset AD at screening/baseline; (21) use of anti-coagulant medication, or known coagulopathy or platelet count <100,000 cells/μL within 4 weeks of the screening visit; (22) treatment with any biologic therapy within five half-lives or 3 months prior to screening, whichever was longer, with the exception of routinely recommended vaccinations, which were allowed; (23) use of anti-seizure medication, anti-Parkinsonian, or stimulant (e.g., methylphenidate) medications; (24) use of investigational drug, device, or experimental medication within 60 days (or five half-lives, whichever is longer) of the screening visit; (25) previous treatment with crenezumab (MABT5102A) or any other therapeutic that targets Ab; (26) history of severe allergic, anaphylactic, or other hypersensitivity reactions to chimeric, human, or humanized antibodies or fusion proteins; (27) contraindication to MRI scan procedures, possibly including medical implants or metal objects, clinically significant claustrophobia, or clinical history or examination finding that would pose a potential hazard in combination with MRI; (28) contraindication to PET scan procedures, possibly including, but not limited to current or recent (within 12 months prior to screening) participation in studies involving radioactive agents, such that the total research-related radiation dose to the participant in any given year would exceed the limits set forth in the U.S. Code of Federal Regulations Title 21 Section 361.1; or (29) abnormal findings that could have affected the participant's response to the radiopharmaceutical and related testing procedures required for PET scans.

The primary objectives of this study were: (1) to evaluate the efficacy of crenezumab treatment compared with placebo for at least 260 weeks based on change in cognitive function as measured by the API ADAD Cognitive Composite Test Battery in preclinical presenilin 1 (PSEN1) E280A autosomal dominant mutation carriers; and (2) to evaluate the efficacy of crenezumab treatment compared with placebo for at least 260 weeks on change in episodic memory function as measured by the FCSRT Cueing Index in preclinical PSEN1 E280A autosomal-dominant mutation carriers.

The secondary objectives of this study were to evaluate PSEN1 E280A mutation carriers for the following: (1) ability of crenezumab to affect clinical endpoints other than those in the primary endpoint family: Alzheimer's Prevention Initiative (API) Autosomal-Dominant Alzheimer's disease (ADAD) Cognitive Composite Test Battery and the FCSRT Cueing Index; (2) ability of crenezumab to reduce cerebral fibrillar amyloid burden in a predefined region of interest (ROI) using florbetapir positron emission tomography (PET); (3) ability of crenezumab to reduce decline in regional cerebral metabolic rate of glucose (CMRg1) using fluorodeoxyglucose (FDG)-PET measurements in a ROI; (4) ability of crenezumab to reduce brain atrophy as measured by volumetric measurements using magnetic resonance imaging (MRI); and (5) ability of crenezumab to affect a tau based cerebrospinal fluid (CSF) biomarker.

The safety objectives for this study were to assess the safety and tolerability of crenezumab in preclinical PSEN1 E280A mutation carriers (comparing crenezumab with placebo). The pharmacokinetic (PK) and pharmacodynamics (PD) objectives for this study were to collect sparse PK samples to support confirmation of exposure to crenezumab and to explore the PD response (as measured by plasma total Ab levels comparing crenczumab with placebo) in preclinical PSEN1 E280A mutation carriers.

The exploratory objectives for this study were the following: (1) assess further the effect of crenezumab in preclinical PSEN1 E280A mutation carriers on additional clinical measures of efficacy and biological markers of disease that have not been pre-specified as primary or secondary endpoints in the Statistical Analysis Plan (SAP); (2) explore pharmacogenetic effects including, but not limited to, a person's apolipoprotein E (APOE) e4 carrier status on the active treatment's cognitive, clinical, and adverse effects; (3) explore effects of genetic variation, including, but not limited to, how genes affect the biology of Alzheimer's disease (AD) and other diseases and how genes influence biomarker responses; (4) examine clinical and biomarker changes in non-carriers and to compare these changes with those seen in carriers treated with placebo; (5) relate the treatment's biomarker effects to clinical outcomes and to examine predictive and prognostic utility of baseline characteristics; and (6) assess the impact of treatment on brain tau load over time. as measured by tau PET imaging in an optional substudy (GN28352-1/BN40199).

Baseline assessments for the identification of outcome measures in accordance with the study objectives as outlined above are summarized in Tables 2-3 below.

TABLE 2
Selected baseline neuropsychiatric ratings* PSEN1 E280A
mutation carriers and non-carriers enrolled in this study.

| | Carriers | Non-Carriers | P-value |
|---|---|---|---|
| N | 167 | 75 | — |
| Neuropsychiatric | 0.46 ± 1.67 | 0.41 ± 2.05 | 0.85 |
| Inventory | | | |
| Geriatric Depression | 1.35 ± 1.83 | 1.19 ± 1.81 | 0.53 |
| Scale | | | |

*Data from 242 age range-matched mutation carriers & non-carriers that are available prior to trial completion. (Tariot et al., (2018) CTAD)

TABLE 3
Selected baseline cognitive test results* for PSEN1 E280A
mutation carriers and non-carriers enrolled in this study.

| | Carriers | Non-Carriers | P-value |
|---|---|---|---|
| MMSE | 28.8 ± 1.4 | 29.2 ± 1.0 | 0.01 |
| CERAD Word List Total Recall | 20.1 ± 4.2 | 21.13 ± 4.0 | 0.07 |
| CERAD Word List Delayed Recall | 6.9 ± 2.2 | 7.7 ± 1.9 | 0.01 |
| RBANS Total Scale | 68.1 ± 12.7 | 74.9 ± 11.8 | <0.001 |
| RBANS Immediate Memory | 87.7 ± 13.5 | 96.9 ± 13.9 | <0.001 |
| RBANS Delayed Memory | 77.4 ± 17.0 | 87.3 ± 12.9 | <0.001 |
| RBANS Attention | 60.5 ± 15.7 | 64.2 ± 14.3 | 0.09 |
| RBANS | 70.9 ± 12.7 | 73.1 ± 12.8 | 0.22 |
| Visuospatial/Constructional | | | |
| RBANS Language | 75.8 ± 18.1 | 80.1 ± 17.2 | 0.08 |
| FCSRT Total | 42.4 ± 6.5 | 44.6 ± 3.2 | <0.001 |
| FCSRT Delayed Recall | 14.2 ± 2.8 | 15.2 ± 1.3 | <0.001 |
| Subjective Memory Checklist- | 9.8 ± 6.3 | 9.2 ± 6.2 | 0.51 |
| Participant | | | |
| Subjective Memory Checklist- | 6.2 ± 5.5 | 5.6 ± 4.6 | 0.46 |
| Study Partner | | | |

*Data from 242 age range-matched mutation carriers & non-carriers that are available prior to trial completion. (Tariot et al., (2018) CTAD)

### Results

The primary efficacy outcome measures included the annualized rate of change on the API ADAD Composite Cognitive Test total score and the annualized rate of change on the FCSRT Cueing Index. The results as shown in Table 4 demonstrate that both the primary endpoints numerically favored crenezumab as compared to the placebo while the data do not show statistically significant benefit of the crenezumab in either endpoint.

TABLE 4
Primary endpoint family: (1) API Composite and (2) FCSRT
Cueing Index in PSEN1 E280A carrier patients

| | API Composite | | FCSRT Cueing Index | |
|---|---|---|---|---|
| | Crenezumab | Placebo | Crenezumab | Placebo |
| | Carrier | Carrier | Carrier | Carrier |
| | (N = 84) | (N = 84) | (N = 84) | (N = 84) |
| Annualized rate of | −1.099 | −1.425 | −0.0317 | −0.0396 |
| change (SE) | (0.291) | (0.291) | (0.0040) | (0.0040) |

| | | |
|---|---|---|
| Difference in | 0.326, (−0.483, 1.134) | 0.0079, (−0.0032, 0.0190) |
| annualized rate of | | |
| change, 95% CI | | |
| *P-value | 0.43 | 0.16 |
| Relative reduction, | 22.9%, (−53.1%, 61.1%) | 19.9%, (−9.3%, 42.2%) |
| 95% CI | | |

As seen above, the treatment of crenezumab in the patients resulted in a reduction of the annualized rate of change on the API ADAD Composite score by 22.9% relative to the treatment of placebo to the patients, i.e., which serves as the reference annualized rate of change on the API ADAD Composite score. This relative reduction is determined as follows:

Relative reduction (%)=−(Difference in annualized rate of change (SE) between the treatments of Crenezumab carrier and Placebo carrier (e.g., 0.326 from Table 4))/Annualized rate of change (SE) of Placebo carrier (e.g., −1.425 from Table 4)×100

Similar to the API ADAD composite endpoint, the data also demonstrated a numerically favorable result to the treatment of crenezumab in the FCSRT Cueing Index by showing the reduction of 19.9% relative to the placebo treatment. This relative reduction is termined as follows:

Relative reduction (%)=−(Difference in annualized rate of change (SE) between the treatments of Crenezumab carrier and Placebo carrier (e.g., 0.0079 from Table 4))/Annualized rate of change (SE) of Placebo carrier (e.g., −0.0396 from Table 4)×100

Unless described otherwise, the same calculation for relative reduction as described above is applied when comparing the results between different patient groups (carriers vs. non-carriers) and/or treatment groups (cren-treated group v. placebo-treat group).

Similar to the outcome in the primary endpoints, the secondary efficacy outcome measures as well as biomarker readouts demonstrated mostly numerically favored results to crenezumab treatment relative to the placebo treatment while no statistically significant benefit was detected. Clinical secondary outcomes included (1) time to progression from preclinical AD to MCI due to AD or from preclinical AD to dementia due to AD; (2) time to progression to non-zero in the Clinical Dementia Rating (CDR) Scale global score; (3) annualized rate of change in the CDR Scale-Sum of Boxes; (4) annualized rate of change in a measure of overall neurocognitive functions using Repeatable Battery for the Assessment of Neuropsychological Status (RBANS). Biomarker outcomes included (1) imaging markers, i.e. annualized rate of change in mean cerebral fibrillary amyloid accumulation using florbetapir PET from a predefined ROI, annualized rate of change in regional cerebral metabolic rate of glucose (CMRg1) using FDG-PET in a predefined ROI, and annualized rate of change in volumetric measurements using MRI; and (2) CSF biomarkers.

The safety outcome measures included analysis of the frequency and severity of treatment-emergent adverse events (AEs) and serious adverse events (SAEs), withdrawals due to AEs, incidence of treatment-emergent amyloid-related imaging abnormalities (ARIA), incidence of cerebral macrohemorrhages, incidence of pneumonia, incidence of injection reactions and infusion-related reactions (IRRs), and incidence of anti-crenezumab antibodies. ARIAs included ARIA-E, for edema or effusion (e.g. cerebral vasogenic edema (VE) or sulcal effusion), and ARIA-H, for hemosiderin deposition (e.g. superficial central nervous system (CNS) siderosis or cerebral microhemorrhages). AEs were graded according to severity using the National Cancer Institute Common Terminology for Adverse Events, Version 4.0 (NCI CTAE v4.0). An Independent Data Monitoring Committee (iDMC) formerly known as the Data and Safety Monitoring Board (DSMB) was used for this study. Results from this study showed that crenezumab was generally safe and well tolerated, which is consistent with that from other clinical trials of the investigational medicine, with no new safety issues identified.

In addition, the study demonstrated crenezumab PK results that are consistent with historical data for subcutaneous crenezumab and lower than expected exposure for IV crenezumab although limited by small sample size.

Additional exploratory outcome measures of interest included clinical indicators, fluid biomarkers, imaging biomarkers, and other outcome measures. Clinical indicators included changes from baseline over time in the following cognitive measures: Trail Making Test (Armitage, Psychological Monographs, 1946; 60: i-48), Mini-Mental State Exam (MMSE) (Folstein et al., J. Psychiatr. Res., 1975; 12:189-198), Repeatable Battery for the Assessment of Neuropsychological Status (RBANS), Index Scores (Randolph, Repeatable Battery for the Assessment of Neuropsychological Status. San Antonio, TX: The Psychological Corporation, 1998), scores of each of the components of the API ADAD Composite Cognitive Test Battery, Preclinical Alzheimer's Cognitive Composite (PACC) (Donohue et al., JAMA Neurology, 2014; 71:961-970), which for this trial will include the FCSRT free and cued recall, MMSE, RBANS story recall, and RBANS coding score. Additionally, clinical endpoints not examined in secondary outcome measures were explored and assessed: changes in the Neuropsychiatric Inventory (NPI) (Cummings et al., Neurology, 1994; 44:2308-2314 and Cummings, Neurology, 1997; 48: S10-S16) total score, items, and factors, changes in the Geriatric Depression Scale (GDS) (Sheikh and Yesavage, Clinical Gerontologist: J Aging Mental Health, 1986; 5:165-173) total score, changes in Functional Assessment Staging of Alzheimer's Disease (FAST) (Sclan and Reisberg, Int Psychogeriatr, 1992; 4:55-69) total score, and changes in Subjective Memory Checklist (Acosta-Baena et al., Lancet Neurol, 2011; 10:213-220). Additional outcomes measured include: changes in primary, secondary, and exploratory outcomes in mutation non-carriers treated with placebo; comparisons of clinical and biomarker outcomes between carriers treated with placebo and non-carriers; changes in primary, secondary, and exploratory outcomes in carriers and non-carriers as functions of APOE genotype and other genetic variations; short-term changes in imaging measures as functions of initiation (e.g., baseline to 12 weeks); and analyses of outcome measures in relation to one another and in relation to baseline characteristics.

### Example 2: the Alzheimer's Prevention Initiative Autosomal Dominant Alzheimer's Disease Colombia Trial

### Study Design and Objectives

This clinical study was comprised of two study periods, termed Study Periods A and B. Study Period A was a prospective, randomized, double-blind, placebo-controlled, parallel-group study of crenezumab versus placebo treatment in carriers of PSEN1 E280A in autosomal-dominant, PSEN1-mutation-causing early-onset AD who did not meet criteria for mild cognitive impairment (MCI) due to AD or dementia due to AD and were, thus, in a preclinical phase of AD. The study also incorporated administration of placebo to individuals who were not PSEN1 E280A autosomal-dominant mutation carriers.

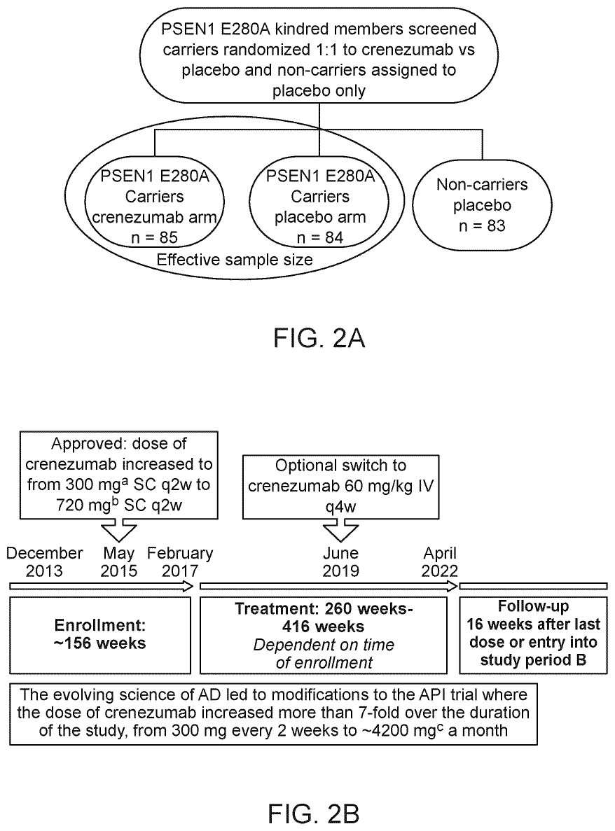

PSEN1 E280A autosomal-dominant mutation carriers who met study eligibility criteria were randomized in a 1:1 ratio to one of two treatment groups: crenezumab or placebo. The study was originally powered to compare the mean change from baseline over 260 weeks in the API ADAD Composite Cognitive Test total score between the active group and the placebo. Assuming a 25% dropout rate, two-sided testing at an overall 0.05 level, a placebo group CV of 65% for the week 260 change scores (=100%×standard deviation of placebo participant change scores/mean of placebo participant change scores) and 100 participants per arm, the study would have at least 80% power to detect a true effect of 30% reduction of the mean decline in the placebo group. In order to maintain genotype blinding and to have a genetic kindred control, a cohort of PSEN1 E280A mutation non-carrier kindred family members were also enrolled in the study and dosed only with placebo. The study included three arms; two arms of participants with a PSEN1 E280A mutation randomized in a 1:1 ratio to active (n=85) or placebo (n=84) and a third arm of non-carriers randomized to placebo (n=84) (see FIG. 2A).

Participants received the randomized treatment until the final participant had reached their final treatment visit at 260 weeks. The study duration for individual participants was at least approximately 284 weeks, including an 8-week screening period, a double-blind treatment period of at least 260 weeks in length, a 4-week final dose visit, and a final safety follow-up visit 16 weeks after the last dose of study drug (crenezumab or placebo) that allowed for clinical follow-up after treatment discontinuation for participants who did not to continue with study drug beyond the end of Study Period A or who terminated study drug early (see FIG. 2B).

Crenezumab was administered either subcutaneously every two weeks or intravenously (60 mg/kg every 4 weeks (4200 mg for an average 70 kg person)), and matching placebo was administered by the same routes at the same frequencies. The evolving science of AD led to modifications of the API trial. During the enrollment period, the approved subcutaneous dose of crenezumab increased from 300 mg (given as two 1 mL subcutaneous injections) every two weeks to 720 mg (given as two 2.2 mL subcutaneous injections) every 2 weeks. The switch to the higher intravenous dose (approximately 4-fold higher exposure to crenczumab) was optional. Participants could decide whether to change from the initial subcutaneous dosing to the IV dosing; however, once intravenous dosing in a given participant had occurred, participants could not switch back to the lower subcutaneous dose.

Following the completion of Study Period A. participants were offered the opportunity to continue in Study Period B. This second study period involved participants continuing to receive study drug until the results of the study were known and post-trial access to crenezumab was started via an open label extension (OLE) or other program, or development of crenczumab was discontinued. All PSEN1 E280A mutation carriers were provided crenezumab in Study Period B regardless of treatment allocation during Study Period A, and all non-carriers continued on placebo. Treatment allocation for both study periods A and B remained blinded. Study Period B lasted up to approximately 9-12 months based on when participants entered Study Period B.

The target population included individuals who were members of a PSEN1 E280A mutation carrier kindred and agreed to conditions of, and were willing to undergo, genetic testing (e.g., APOE, PSEN1 E280A, and other genetic testing) to have PSEN1 E280A mutation carrier or non-carrier status confirmed prior to or during the screening period. Baseline patient data is included below in Table 5. Patients were males and females of the age range ≥30 years and ≤60 years; had MMSE of ≥24 for participants with less than 9 years of education or MMSE of ≥26 for participants with 9 or more years of education; did not meet criteria for dementia due to AD; did not meet criteria for MCI due to AD as defined by the following: (a) cognitive concern based in part on the average Subjective Memory Checklist score >22 (average of participant and study partner component scores), (b) Word List: Recall <3 for participants with less than 9 years of education, (c) Word List: Recall <5 for participants with 9 or more years of education, and (d) preservation of independence in functional activities based in part on review of the Functional assessment staging (FAST); demonstrated adequate vision and hearing to be able to complete testing; had a study partner who agreed to participate in the study and was capable of and willing to: (a) accompany the participant to all required visits for subcutaneous administration or intravenous administration, (b) provide information for required telephone assessments, (c) spend sufficient time with the participant to be familiar with his/her overall function and behavior and be able to provide adequate information about the participant including: (i) knowledge about domestic activities, hobbies, routines, social skills and basic activities of daily life, (ii) work and educational history. (iii) cognitive performance, including memory abilities, language abilities, temporal and spatial orientation, judgment, and problem solving, (iv) emotional and psychological state, and (v) general health status. The participant and study partner had evidence of the following: (a) adequate premorbid functioning (e.g., intellectual, visual, and auditory), and (b) fluency in, and able to read, the language in which study assessments were administered. Participants were willing and able to undergo neuroimaging (PET and MRI); displayed no clinically significant thyroid dysfunction or B12 deficiency as determined by the following criteria: (a) serum thyroid stimulating hormone (TSH) and B12 levels within normal or expected ranges for the testing laboratory, (b) if the participant was undergoing thyroid replacement therapy, TSH levels were either within normal or expected ranges for the testing laboratory, or (c) if the participant was receiving vitamin B12 injections or oral vitamin B12 therapy, B12 levels were at or above the lower limit of normal for the testing laboratory; and were in good general health with no known co-morbidities expected to interfere with participation in the study. At baseline, the carrier population had a mean age of approximately 37, approximately 7 years younger than the median age of MCI onset; and nearly 50% had a negative Aβ PET scan.

TABLE 5
Participant Baseline Characteristics

| | Crenezumab- | Placebo- | Placebo- |
|---|---|---|---|
| | Carrier | Carrier | Non-carrier |
| | (n = 85) | (n = 84) | (n = 83) |
| Age, mean (SD)* | 36.8 (5.3) | 36.9 (6.3) | 43.3 (7.2) |
| Female sex | 51.8% | 70.2% | 68.7% |
| Education >=9 years* | 56.5% | 56.0% | 45.8% |
| >=1 APOE4 allele* | 22.4% | 20.2% | 22.9% |
| CDR-GS = 0* | 90.6% | 88.1% | 94.0% |
| CDR-SB, mean (SD) | 0.16 (0.38) | 0.14 (0.43) | 0.05 (0.17) |
| API Composite, | 81.9 (8.8) | 80.4 (11.3) | 83.7 (9.8) |
| mean (SD) | | | |
| FCSRT CI, mean (SD) | 0.78 (0.16) | 0.76 (0.20) | 0.83 (0.14) |
| MMSE Total Score, | 28.9 (1.3) | 28.8 (1.5) | 29.2 (1.0) |
| mean (SD) | | | |
| NPI, mean (SD) | 0.26 (0.89) | 0.64 (2.16) | 0.37 (1.95) |
| FAST, mean (SD) | 1.09 (0.29) | 1.13 (0.37) | 1.01 (0.11) |
| RBANS total | 68.4 (11.8) | 67.8 (13.5) | 74.8 (11.5) |
| ( score—mean SD) | | | |
| \hat{ }Amyloid | 61.2% | 48.8% | 0 |
| PET Positive, | 1.15 (0.15) | 1.11 (0.12) | 0.96 (0.04) |
| Amyloid PET SUVr, | | | |
| mean (SD) | | | |
| Amyloid PET | 33.41 (27.24) | 25.36 (21.43) | −2.18 (8.23) |
| centiloids—mean (SD) | | | |
| FDG PET, | 1.29 (0.04) | 1.29 (0.04) | 1.30 (0.04) |
| SUVR—mean (SD) | | | |
| Whole brain vMRI—ml, | 1187.65 | 1150.80 (89.91) | 1144.84 |
| mean (SD) | (116.14) | | (112.44) |
| Hippocampal | 8.38 (0.85) | 8.24 (0.73) | 8.24 (0.76) |
| vMRI—ml, mean (SD) | | | |
| Lateral ventricles vMRI | 13.34 (6.14) | 13.39 (5.64) | 14.14 (6.75) |
| ml, mean (SD) | | | |
| CSF pTau181—no. | 48 | 42 | 37 |
| CSF pTau181—pg/ml, | 20.23 (18.36) | 19.13 (11.84) | 11.02 (5.18) |
| mean (SD)‡ | | | |
| CSF tTau—no. | 48 | 42 | 36 |
| CSF Tau, pg/ml, | 197.91 (123.94) | 202.95 (95.08) | 137.61 (58.32) |
| mean (SD)‡ | | | |
| CSF NfL—no. | 48 | 42 | 37 |
| CSF NfL | 1.71 (0.33) | 1.72 (0.20) | 1.66 (0.15) |
| (Log₁₀)—pg/ml, | | | |
| mean (SD)‡ | | | |

Data on file. *stratification variables, \hat{ }Whole cerebellum used as the reference region; threshold >1.1 defined as positive.
‡A subset of participants took part in the optional CSF substudy.

API Alzheimer's Prevention Initiative, APOE 84 apolipoprotein E 84 allele, CDR-GS Clinical Dementia Rating-Global Score, CDR-SB Clinical Dementia Rating-Sum of Boxes, CSF cerebrospinal fluid, FCSRT-CI Free and Cued Selective Reminding Test-Cueing Index, FDG fluorodeoxyglucose, GTP1 Genentech Tau Probe 1, ITT intention-to-treat, MMSE Mini-Mental State Examination, NfL neurofilament light chain, NPI Neuropsychiatric Inventory, PET positron emission tomography, pTau181 phosphorylated tau at threonine 181, RBANS Repeatable Battery for the Assessment of Neuropsychological Status, SD standard deviation, SUVR standardized uptake value ratio, tTau total tau, vMRI volumetric (i.e., T1-weighted) magnetic resonance imaging.

The target population excluded anyone with (1) significant medical, psychiatric, or neurological condition or disorder documented by history, physical, neurological, laboratory, or ECG examination that would place the participant at undue risk or impact the interpretation of efficacy; (2) history of stroke; (3) history of severe, clinically significant CNS trauma (e.g., cerebral contusion); (4) body weight <45 or >120 kg; (5) history or presence of atrial fibrillation that posed a risk for future stroke; (6) clinically significant laboratory or ECG abnormalities; (7) presence of bipolar disorder or other clinically significant major psychiatric disorder according to DSM-IV-TR (recorded as an AE if diagnosed after study enrollment); (8) clinically significant depression, based in part by a Geriatric Depression Scale (short form GDS) (15-point scale) score >9 at screening; (9) history of seizures; (10) myocardial infarction within 2 years, congestive heart failure, atrial fibrillation, or uncontrolled hypertension; (11) history of cancer within 5 years; (12) clinically significant infection within the last 30 days prior to screening; (13) brain MRI imaging results at baseline showing any of the following: (a) evidence of any ARIA-E (cerebral VE, sulcal effusion), infection, significant cerebral vascular pathology, clinically significant lacunar infarct in a region important for cognition or multiple lacunes or a cortical infarct or focal lesions of clinical significance, (b) more than four cerebral microhemorrhages (lesions with diameter ≤10 mm), regardless of their anatomical location or diagnostic characterization as “possible” or “definite”, or (c) single area of superficial siderosis of the CNS or evidence of a prior cerebral macrohemorrhage (lesion with diameter >0 mm); (14) clinically significant screening blood or urine laboratory abnormalities requiring further evaluation or treatment, including: (a) impaired hepatic function, as indicated by transaminases >2 × the upper limit of normal (ULN) or clinically significant abnormalities in synthetic function tests, (b) impaired coagulation (aPTT >1.2 × ULN), (c) platelet count <100,000/μL, or (d) glycosylated hemoglobin >8.0%; (15) positive urine test for drugs of abuse at screening (results of cannabinoid assays were not used for the determination of eligibility); (16) history of alcohol or substance dependence within the previous two years (DSM-IV TR criteria); (17) use of any other medications with the potential to significantly affect cognition (including, but not limited to, sedatives, narcotics (e.g., opiates/opioids), hypnotics, over-the-counter (OTC) sleeping aids, sedating anti-allergy medications); (18) use of typical anti-psychotics or barbiturates; (19) use of non-anti-cholinergic antidepressant medications or atypical anti-psychotics unless maintained on a stable dose regimen for at least 6 weeks prior to screening; (20) use of any FDA/INVIMA-approved medications for treatment of late-onset AD at screening/baseline; (21) use of anti-coagulant medication, or known coagulopathy or platelet count <100,000 cells/μL within 4 weeks of the screening visit; (22) treatment with any biologic therapy within five half-lives or 3 months prior to screening, whichever was longer, with the exception of routinely recommended vaccinations, which were allowed; (23) use of anti-seizure medication, anti-Parkinsonian, or stimulant (e.g., methylphenidate) medications; (24) use of investigational drug, device, or experimental medication within 60 days (or five half-lives, whichever is longer) of the screening visit; (25) previous treatment with crenezumab (MABT5102A) or any other therapeutic that targets Ab; (26) history of severe allergic, anaphylactic, or other hypersensitivity reactions to chimeric, human, or humanized antibodies or fusion proteins; (27) contraindication to MRI scan procedures, possibly including medical implants or metal objects, clinically significant claustrophobia. or clinical history or examination finding that would pose a potential hazard in combination with MRI; (28) contraindication to PET scan procedures, possibly including, but not limited to current or recent (within 12 months prior to screening) participation in studies involving radioactive agents, such that the total research-related radiation dose to the participant in any given year would exceed the limits set forth in the U.S. Code of Federal Regulations Title 21 Section 361.1; (29) abnormal findings that could have affected the participant's response to the radiopharmaceutical and related testing procedures required for PET scans; (30) pregnancy.

The primary objectives of this study were: (1) to evaluate the efficacy of crenezumab treatment compared with placebo for at least 260 weeks based on change in cognitive function as measured by the API ADAD Cognitive Composite Test Battery in preclinical presenilin 1 (PSEN1) E280A autosomal dominant mutation carriers; and (2) to evaluate the efficacy of crenczumab treatment compared with placebo for at least 260 weeks on change in episodic memory function as measured by the FCSRT Cueing Index in preclinical PSEN1 E280A autosomal-dominant mutation carriers.

The secondary objectives of this study were to evaluate PSEN1 E280A mutation carriers for the following: (1) ability of crenezumab to affect clinical endpoints other than those in the primary endpoint family: Alzheimer's Prevention Initiative (API) Autosomal-Dominant Alzheimer's disease (ADAD) Cognitive Composite Test Battery and the FCSRT Cueing Index; (2) ability of crenezumab to reduce cerebral fibrillar amyloid burden in a predefined region of interest (ROI) using florbetapir positron emission tomography (PET); (3) ability of crenezumab to reduce decline in regional cerebral metabolic rate of glucose (CMRg1) using fluorodeoxyglucose (FDG)-PET measurements in a ROI; (4) ability of crenezumab to reduce brain atrophy as measured by volumetric measurements using magnetic resonance imaging (MRI); and (5) ability of crenezumab to affect a tau based cerebrospinal fluid (CSF) biomarker.

The safety objectives for this study were to assess the safety and tolerability of crenezumab in preclinical PSEN1 E280A mutation carriers (comparing crenezumab with placebo). The pharmacokinetic (PK) and pharmacodynamics (PD) objectives for this study were to collect sparse PK samples to support confirmation of exposure to crenezumab and to explore the PD response (as measured by plasma total Ab levels comparing crenezumab with placebo) in preclinical PSEN1 E280A mutation carriers.

The exploratory objectives for this study were the following: (1) assess further the effect of crenezumab in preclinical PSEN1 E280A mutation carriers on additional clinical measures of efficacy and biological markers of disease that have not been pre-specified as primary or secondary endpoints in the Statistical Analysis Plan (SAP); (2) explore pharmacogenetic effects including, but not limited to, a person's apolipoprotein E (APOE) e4 carrier status on the active treatment's cognitive, clinical, and adverse effects; (3) explore effects of genetic variation, including, but not limited to, how genes affect the biology of Alzheimer's disease (AD) and other diseases and how genes influence biomarker responses; (4) examine clinical and biomarker changes in non-carriers and to compare these changes with those seen in carriers treated with placebo; (5) relate the treatment's biomarker effects to clinical outcomes and to examine predictive and prognostic utility of baseline characteristics; and (6) assess the impact of treatment on brain tau load over time, as measured by tau PET imaging in an optional substudy (GN28352-1/BN40199).

### Results

Table 6 shows the patient adherence and retention rates over the 8-year study. 94.0% of all participants completed study period A and 90.5% percent of all participants completed treatment in period A. Treatment discontinuations were chiefly due to participant decision (n=12), adverse events (n=5), and pregnancy (n=4).

TABLE 6
Participant Disposition

| | Crenezumab- | Placebo- | Placebo- | All |
|---|---|---|---|---|
| | Carrier | Carrier | Non-carrier | participants |
| | (n = 85) | (n = 84) | (n = 83) | (N = 252) |
| Completed | 79 (92.9 %) | 78 (92.9 %) | 80 (96.4 %) | 237 (94.0 %) |
| study | | | | |
| period A | | | | |
| Completed | 76 (89.4 %) | 75 (89.3 %) | 77 (92.8 %) | 228 (90.5 %) |
| treatment | | | | |
| in period A | | | | |

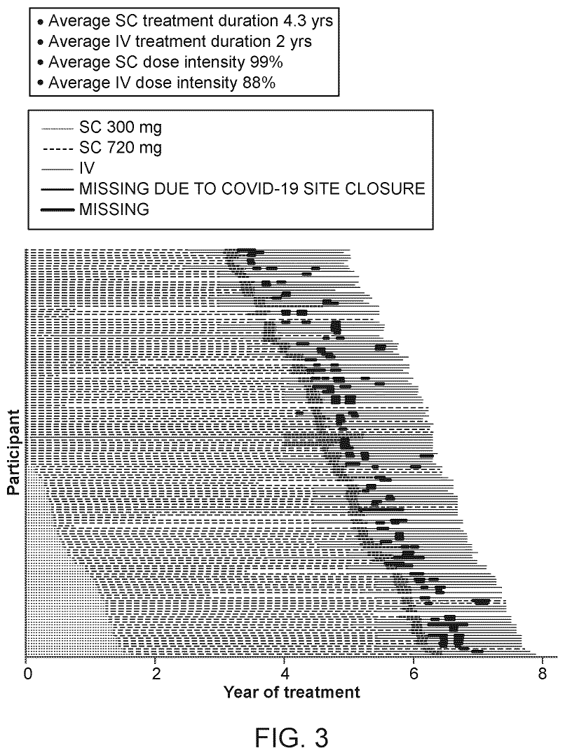

The mean length of treatment of patients receiving at least one dose of the study drug was 6.1 years, with the longest treatment period being 7.9 years. The average duration of subcutaneous treatment was 4.3 years, with an average dose intensity of 99%. The average duration of intravenous treatment was 2 years, with an average dose intensity of 88%. As shown in FIG. 3, a portion of the patients began with a 300 mg subcutaneous dose before switching to a 720 mg dose; some patients remained on the 300 mg subcutaneous dose, or switched to the 300 mg dose after beginning at 720 mg.

Outcomes were analyzed using the random coefficient regression model (RCRM) in mutation carriers receiving at least one dose of the study drug, which provides a simple and holistic measure of the average clinical benefit over the full duration of the trial. See, e.g., Hu et al., Biom J. 2021; 63:806-24, which is hereby incorporated by reference in its entirety. The RCRM adjusts for age, education, APOE4 and CDR-GS at baseline, and adjusts for treatment assignment for slope; both random intercept and slope terms are added to the model.

TABLE 7
Results for primary outcomes (mutation carriers only)

| | API Composite | | FCSRT Cueing Index | |
|---|---|---|---|---|
| | (Range 0-100) | | (Range (0-1) | |
| | Crenezumab | Placebo | Crenezumab | Placebo |
| | Carrier | Carrier | Carrier | Carrier |
| | (N = 84) | (N = 84) | (N = 84) | (N = 84) |
| Annualized rate of | −1.10 | −1.43 | −0.03 | −0.04 |
| change (SE) points | (0.29) | (0.29) | (0.004) | (0.004) |
| per year | | | | |

| | | |
|---|---|---|
| Difference in | 0.33, (−0.48, 1.13) | 0.008, (−0.003, 0.019) |
| annualized rate of | | |
| change, 95% CI | | |
| P-value | 0.43 | 0.16 |
| Relative reduction, | 22.9%, (−53.1%, 61.1%) | 19.9%, (−9.3%, 42.2%) |
| 95% CI* | | |

CI, confidence interval; SE standard error.
*95% CI for relative reduction is based on the bootstrap method with replacement

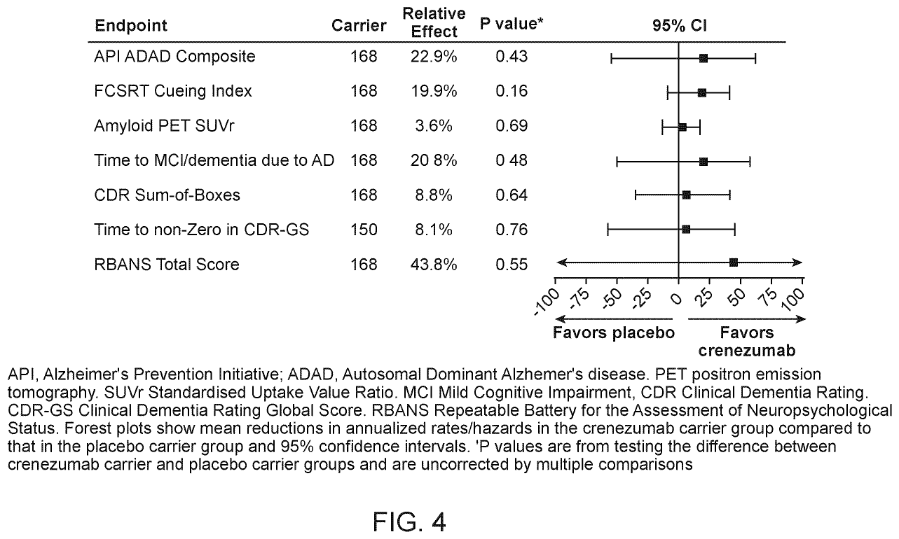

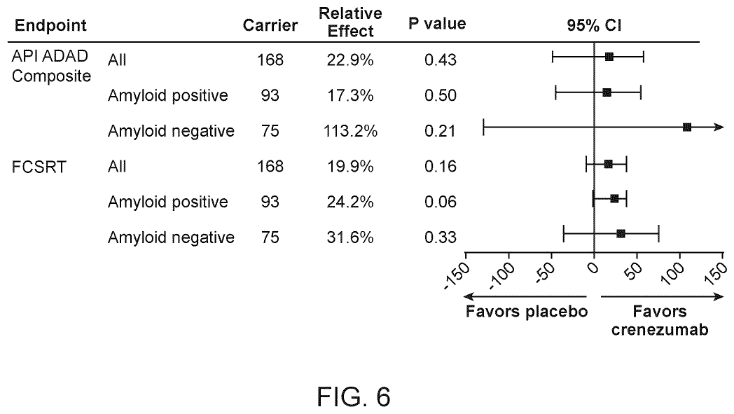

As shown in Table 7, FIG. 4, and FIG. 6, the treatment of crenezumab in the patients resulted in a reduction of the annualized rate of change on the API ADAD Composite score by 22.9% relative to the treatment of placebo to the patients, i.e., which serves as the reference annualized rate of change on the API ADAD Composite score. This relative reduction is determined as follows: Relative reduction (%)=−(Difference in annualized rate of change (SE) between the treatments of Crenezumab carrier and Placebo carrier (e.g., 0.326 from Table 4))/Annualized rate of change (SE) of Placebo carrier (e.g., −1.425 from Table 4)×100. FIG. 6 shows a breakdown of the API ADAD Composite by baseline amyloid status, with amyloid positive patients having a reduction of the annualized rate of change of 17.3% relative to placebo, while in amyloid negative patients, the reduction was 113.2%. Similar to the API ADAD composite endpoint, the data also demonstrated a numerically favorable result to the treatment of crenezumab in the FCSRT Cueing Index by showing the reduction of 19.9% relative to the placebo treatment. This relative reduction is termined as follows: Relative reduction (%)=−(Difference in annualized rate of change (SE) between the treatments of Crenezumab carrier and Placebo carrier (e.g., 0.0079 from Table 4))/Annualized rate of change (SE) of Placebo carrier (e.g., −0.0396 from Table 4)×100. FIG. 6 shows a breakdown of the FCSRT by baseline amyloid status, with amyloid positive patients having a reduction of 24.2% relative to placebo, while in amyloid negative patients, the reduction was 31.6%.

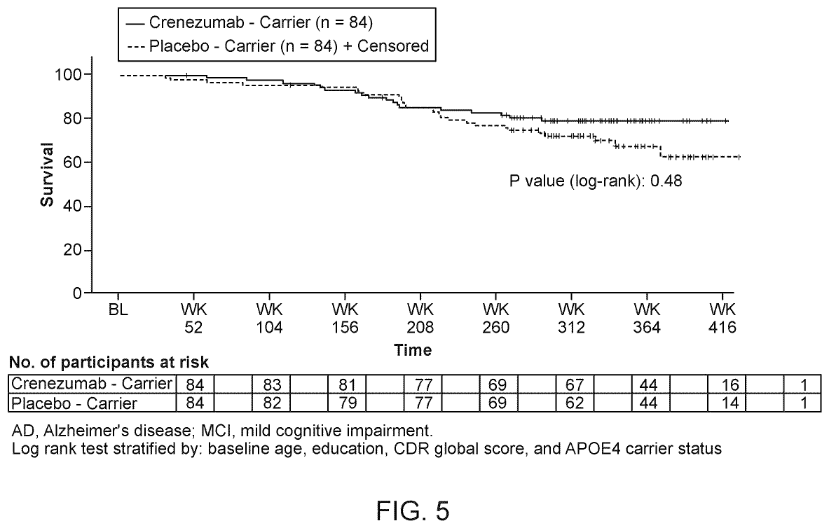

As shown in FIG. 4, the secondary efficacy outcome measures as well as biomarker readouts numerically favored results to crenezumab treatment relative to the placebo treatment, although no statistically significant benefit was detected. Clinical secondary outcomes included (1) amyloid PET SUVr (standardized uptake value ratio); (2) time to progression from preclinical AD to MCI due to AD or from preclinical AD to dementia due to AD (see also FIG. 5 showing the Kaplan-Meier curve for time to MCI or dementia due to AD); (3) annualized rate of change in the CDR Scale-Sum of Boxes; (4) time to progression to non-zero in the Clinical Dementia Rating (CDR) Scale global score; (5) annualized rate of change in a measure of overall neurocognitive functions using Repeatable Battery for the Assessment of Neuropsychological Status (RBANS).

Brain images and biological samples were collected as indicated in Table 8, and specific biomarker modalities and measurements are indicated in Table 9. All participants had serial amyloid-β PET, FDG PET, and MRI scans and provided annual blood samples. Half of the participants had serial Tau PET scans (introduced later in the study). Approximately half of the participants had one or more lumbar punctures.

TABLE 8
Brain images and biological samples

| Brain images and samples | Visits |
|---|---|
| Amyloid-β (Florbetapir) PET | BL, 24 and 60 mos, and last |
| Tau (GTP1) PET, optional | 32-52 mos, 45-67 mos, and last |
| FDG PET | BL, 3, 24 and 60 mos, and last |
| Volumetric and other MRIs | BL, 3, 6, 9, 12 mos, every 6 mos, and last |
| CSF samples, optional | BL, 24 and 60 mos, and last |
| Blood samples | BL, annual, and last |

TABLE 9
Biomarker measurements

| Modality | Measurements |
|---|---|
| Florbetapir | mean cortical-to-cerebellar SUVRs for baseline |
| (Aβ) PET | comparisons; mean cortical-white matter SUVRs for |
| | longitudinal comparisons |
| GTP1 (Tau) PET, | entorhinal cortex (ERC) SUVRs |
| optional | |
| FDG PET | SUVRs (CMRgl decline) in an AD-related statistical |
| | ROI (sROI) |
| Volumetric and | hippocampal, ventricular and whole brain volume |
| other MRIs | |
| CSF biomarkers | Aβ42, Aβ40, pTau181, tTau, NfL, & neurogranin |
| | (A/T/N); GFAP, sTREM2, YKL-40, IL6, s100b |
| | (neuroinflammation); a-synuclein; oligomeric AB |
| Plasma biomarkers | pTau181, pTau217 & NfL (A/T/N); GFAP, sTREM2, |
| | YKL-40 (neuroinflammation) |

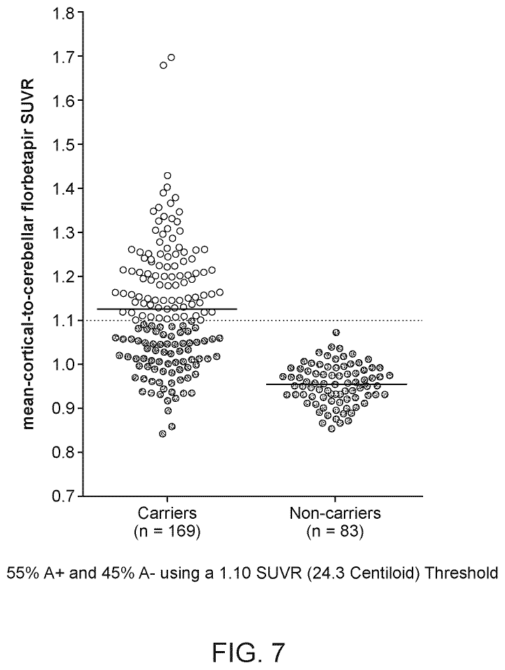

FIG. 7 shows differences in the baseline Aβ in the PSEN1 E280A mutation carrier and non-carrier groups. 55% of carriers had the E280A mutation (empty circles) and 45% were A-(speckled circles). The results show there were approximately twice as many A-carriers than expected. The unexpectedly early AD stage of carriers led to less progression in the placebo group and contributed to reduced power in detecting significant slowing in the treatment group. These data, however, provide a chance to explore the treatment effects in A+ and A− carriers and inform the design, size, selection criteria, and endpoints in future secondary and primary prevention trials.

Table 9 shows baseline brain imaging measurements. Baseline entorhinal cortex GTP1 SUVRs are not available since GTP PET was introduced later in the trial. Table 10 shows baseline CSF biomarker measurements. CSF biomarker measurements were performed using Roche's Elecsys platform. YKL-40 and S100b concentrations are in μg/mL, all other concentrations are in pg/mL. P-values are uncorrected for multiple comparisons. Sample size includes all participants who received at least one dose of the study drug and who had at least one CSF measurement at baseline.

TABLE 9
Baseline brain imaging measurements

| | Carriers | Non-carriers | Nominal |
|---|---|---|---|
| | (n = 168) | (n = 83) | P-value |
| Biomarker | Mean (SD) | Mean (SD) | (age adjusted) |
| Aβ plaque | 1.13 (0.14) | 0.96 (0.04) | <0.0001 |
| deposition* | | | |
| CMRgl in a | 1.29 (0.04) | 1.30 (0.04) | <0.0001 |
| statistical ROI** | | | |
| Hippocampal | 8,451 (815) | 8,361 (788) | 0.67 |
| volume (ml)\hat{ } | | | |

*SPM-based mean cortical-to-cerebellar florbetapir SUVRs;
**SPM-based FDG SUVRs;
\hat{ }Freesurfer-based hippocampal volumes (ml).
P-values are uncorrected for multiple comparisons.
Aβ, amyloid-beta;
CMRgl, cerebral metabolic rate for glucose;
FDG, fluorodeoxyglucose;
GTP1, Genentech Tau Probe 1;
PET, positron emission tomography;
ROI, region of interest;
SD, standard deviation;
SPM, statistical parametric mapping;
SUVR, standard uptake value ratio.

TABLE 10
Baseline CSF biomarker measurements

| | Carriers (n = 90) | Non-carriers (n = 37) | Nominal P-value |
|---|---|---|---|
| Biomarker | Mean (SD) | Mean (SD) | (age adjusted) |
| Aβ42 | 823 (258) | 1,176 (258) | <0.0001 |
| Aβ40 | 13,623 (3,645) | 14,799 (4,282) | 0.46 |
| pTau181 | 21.2 (15.7) | 13.1 (5.1) | <0.0001 |
| Total Tau | 202 (110) | 144 (57) | <0.0001 |
| NfL (Log₁₀) | 1.71 (0.28) | 1.66 (0.15) | 0.0006 |
| Neurogranin | 1,079 (637) | 827 (328) | 0.007 |
| GFAP | 4,715 (2,453) | 4,073 (1,980) | <0.0001 |
| sTREM2 | 7.59 (2.43) | 7.44 (2.11) | 0.05 |
| YKL-40 | 92,352 (46,038) | 90,774 (31,519) | 0.005 |
| IL6 (Log₁₀) | 0.56 (0.16) | 0.55 (0.12) | 0.88 |
| S100b | 0.73 (0.23) | 0.68 (0.28) | 0.16 |
| α-Synuclein | 324 (252) | 252 (152) | 0.06 |

Aβ, amyloid-beta;
CSF, cerebrospinal fluid;
GFAP, glial fibrillary acidic protein;
IL6, interleukin 6;
NfL, neurofilament light chain;
pTau, phosphorylated Tau;
S100b, calcium-binding protein B;
SD, standard deviation;
sTREM2, soluble triggering receptor expressed on myeloid cells 2;
YKL-40, chitinase-3-like protein 1

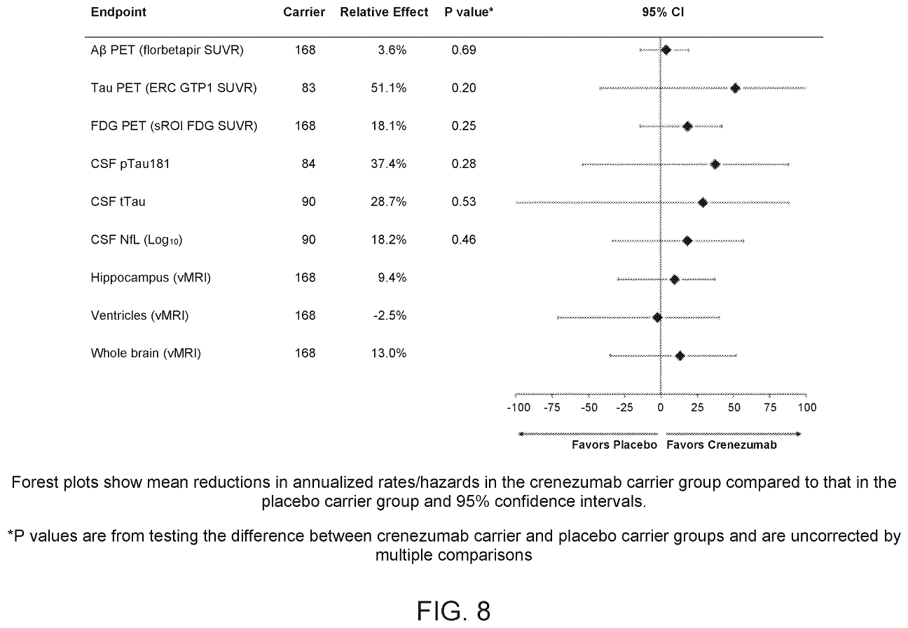

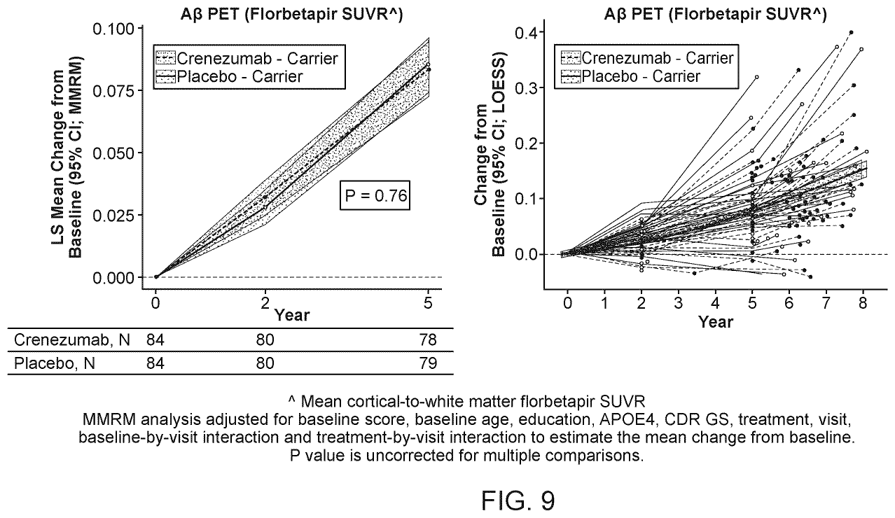

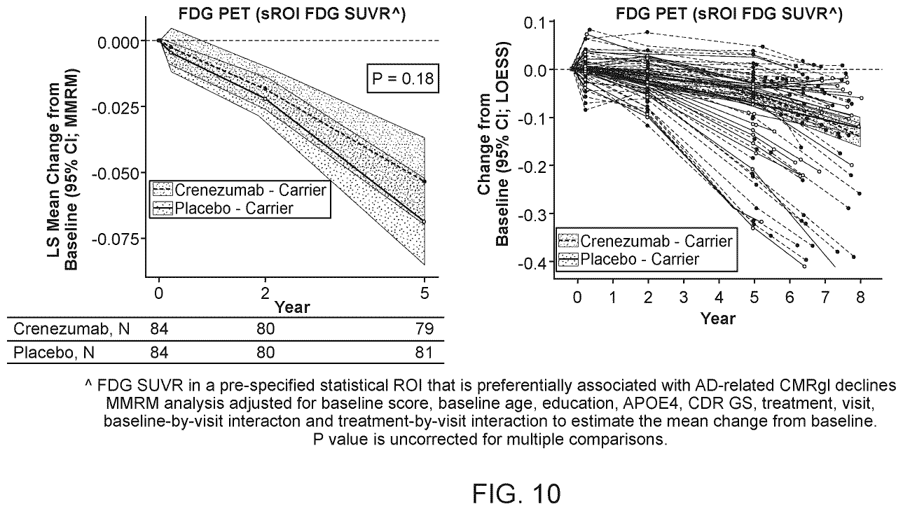

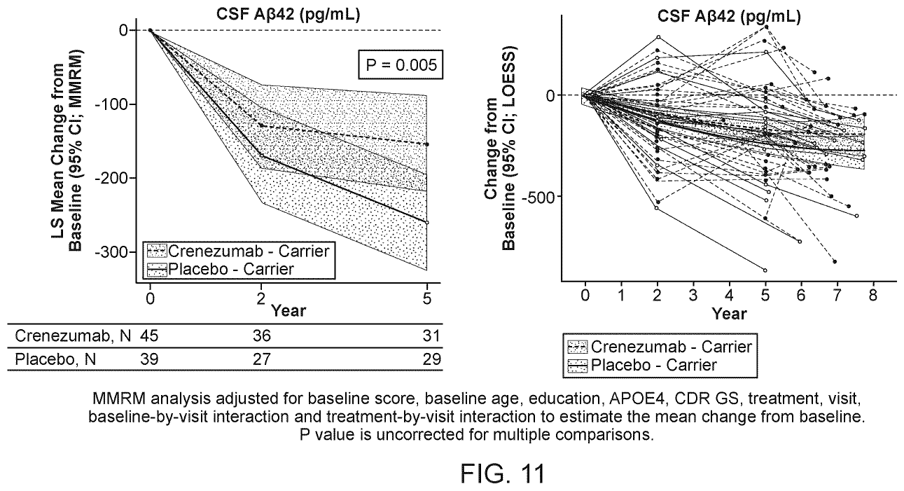

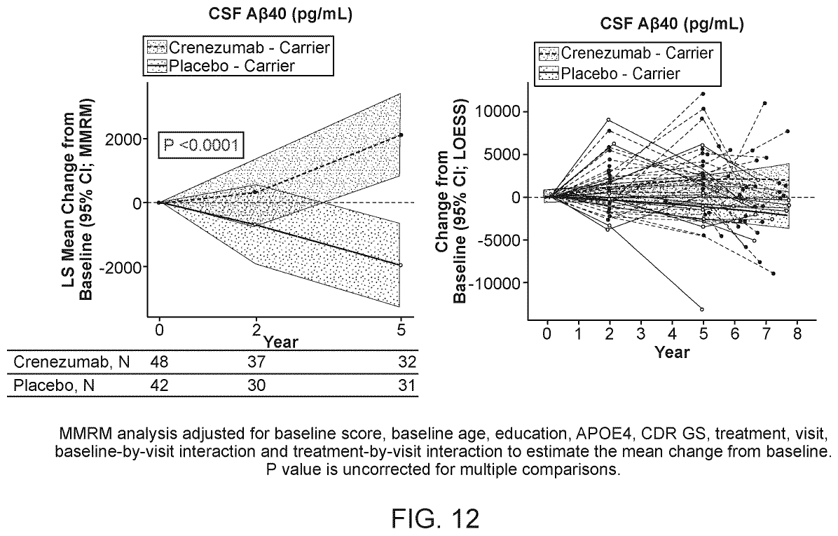

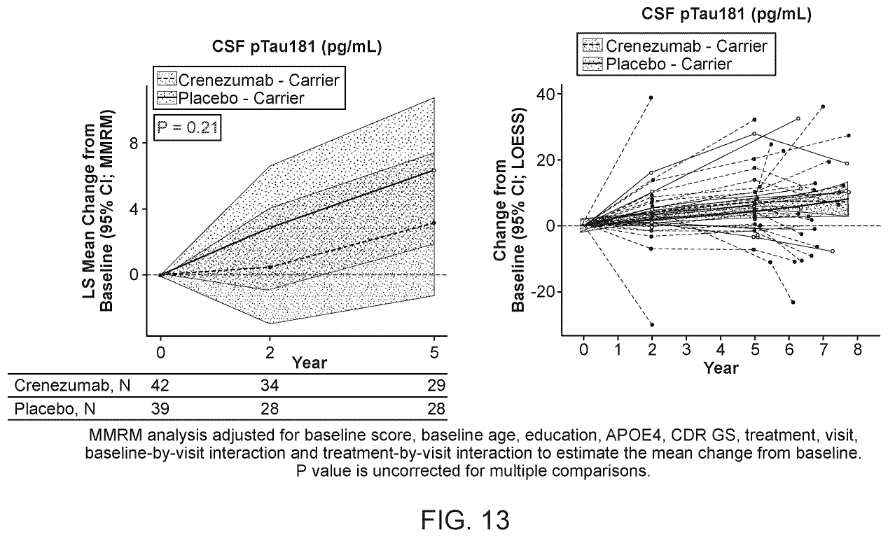

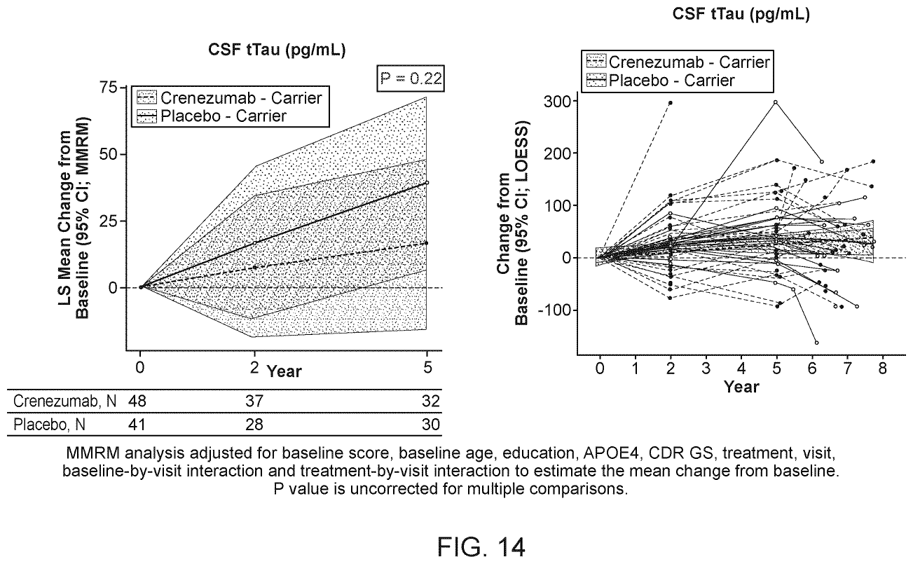

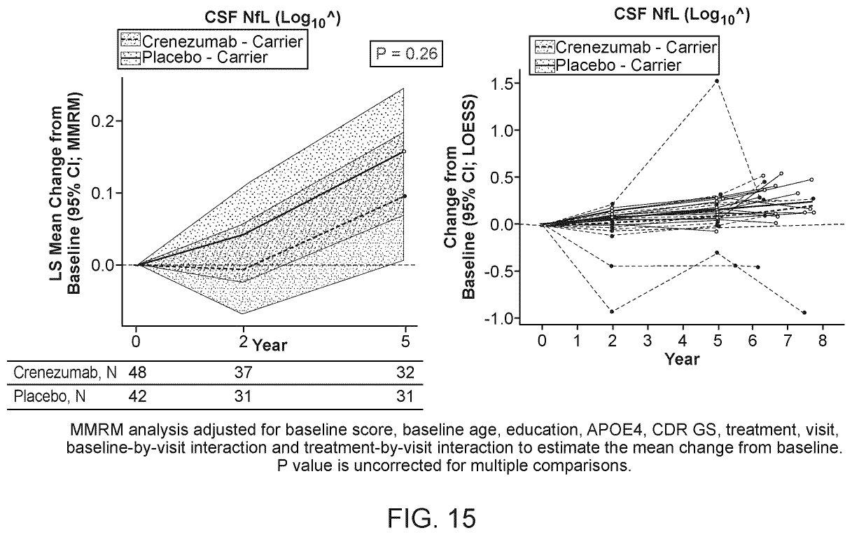

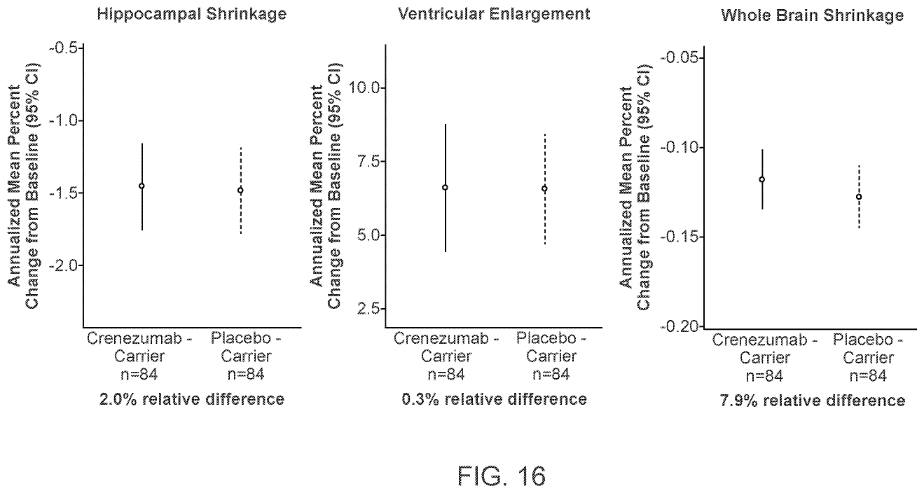

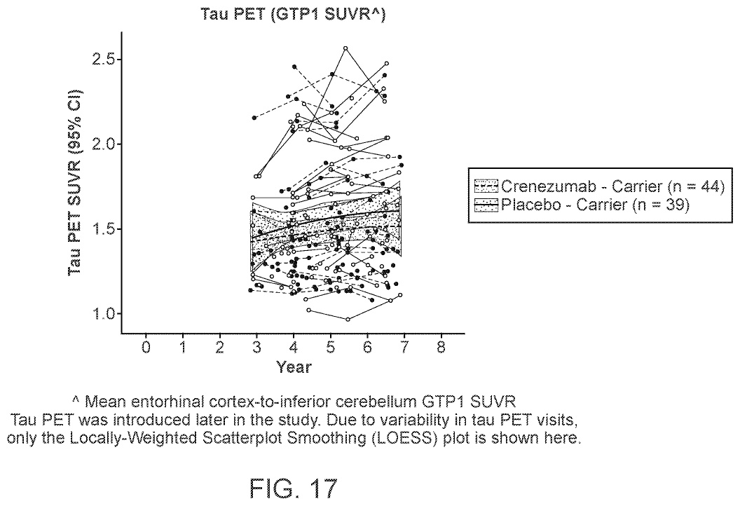

FIGS. 8-17 show additional biomarker outcomes in the crenezumab and placebo-treated PSEN1 E280A mutation carrier groups. In FIG. 8, relative reduction is the mean reduction in annualized rates/hazards in the crenezumab carrier group compared to that in the placebo carrier group. Amyloid-β was increased in both the placebo and crenczumab carrier groups as determined by PET, although no statistical difference was observed between the groups (FIG. 9). FDG declined in both the placebo and crenezumab carrier groups as determined by PET, although no statistical difference was observed between the groups (FIG. 10). CSF Amyloid-B42 declined in the placebo carrier group, but an attenuated decline was observed for the crenezumab carrier group (FIG. 11). An unexpected decline in the placebo carrier group was observed for CSF Aβ40, while an increase was observed for the crenezumab carrier group. FIG. 12 shows an increase in CSF pTau181 in placebo treated carriers, but no statistically significant difference was observed between the crenezumab and placebo carrier groups. CSF total Tau was also increased in the placebo carrier group, no statistically significant difference was observed between the crenezumab and placebo carrier groups (FIG. 13). Further, no statistically significant differences in Tau between the crenezumab and placebo carrier groups was observed by PET (FIG. 17). CSF Neurofilament light (NfL) was increased in both placebo and crenezumab carriers, but no statistically significant differences were observed between the two groups (FIG. 15). Finally, no statistically significant differences were observed between crenezumab and placebo carrier groups in brain volume as measured by MRI although, at least in the measurement of the whole brain shrinkage and hippocampal shrinkage, the cren-treated carrier group shows numerically favored results compared to or relative to the place-treated carrier group (FIG. 16).

The safety outcome measures included analysis of the frequency and severity of treatment-emergent adverse events (AEs) and serious adverse events (SAEs), withdrawals due to AEs, incidence of treatment-emergent amyloid-related imaging abnormalities (ARIA), incidence of cerebral macrohemorrhages, incidence of pneumonia, incidence of injection reactions and infusion-related reactions (IRRs), and incidence of anti-crenezumab antibodies. ARIAs included ARIA-E, for edema or effusion (e.g. cerebral vasogenic edema (VE) or sulcal effusion), and ARIA-H, for hemosiderin deposition (e.g. superficial central nervous system (CNS) siderosis or cerebral microhemorrhages). AEs were graded according to severity using the National Cancer Institute Common Terminology for Adverse Events, Version 4.0 (NCI CTAE v4.0). An Independent Data Monitoring Committee (iDMC) formerly known as the Data and Safety Monitoring Board (DSMB) was used for this study.

As shown in Tables 11-13, results from this study showed safety and tolerability were good, with no new safety issues identified. Moreover, as shown above in Table 4, the study had excellent adherence and retention rates over the 8-year study.

TABLE 11
Overview of adverse events

| | Crenezumab- | Placebo- | Placebo- |
|---|---|---|---|
| | Carrier | Carrier | Non-carrier |
| | n = 85 | n = 84 | n = 83 |
| Participants with at least one event | n (%) | n (%) | n (%) |
| Adverse event (AE) | 84 (100) | 84 (100) | 83 (100) |
| Total number of AEs | 2164 | 2295 | 2367 |
| Serious adverse event (SAE) | 23 (27.4) | 21 (25) | 12 (14.5) |
| Total number of SAEs | 30 | 22 | 14 |
| Fatal AE | 0 | 0 | 0 |
| NCI CTCAE* grade ≥3 | 18 (21.4) | 23 (27.4) | 10 (12.0) |
| Treatment-related AE | 62 (73.8) | 49 (58.3) | 51 (61.4) |
| Treatment-related SAE** | <3 | <3 | <3 |
| AE or SAE leading to treatment | 2 (2.4) | 2 (2.4) | 2 (2.4) |
| withdrawal | | | |

*NCI CTCAE, National Cancer Institute Common Terminology Criteria For Adverse Events;
**formatted to protect treatment blind; adverse events with onset from first dose of study drug up to 16 weeks after last dose in study period A are presented for the safety-evaluable population (n = 251)

TABLE 12
Serious adverse events (n ≥ 3 overall)

| | Crenezumab- | Placebo- |
|---|---|---|
| | Carrier | Carrier |
| MedDRA System Organ | n = 84 | n = 84 |
| Class Preferred Term | n (%) | n (%) |
| Participants with any SAE | 23 (27.4) | 21 (25.0) |
| Total number of SAEs | 30 | 22 |
| Infections and infestations | 5 (6.0) | 5 (6.0) |
| Pregnancy, puerperium and perinatal | 5 (6.0) | 3 (3.6) |
| conditions | | |
| Injury, poisoning and procedural | 3 (3.6) | 2 (2.4) |
| complications | | |
| Psychiatric disorders | 2 (2.4) | 1 (1.2) |
| Reproductive system and breast disorders | 2 (2.4) | 2 (2.4) |
| Gastrointestinal disorders | 2 (2.4) | 1 (1.2) |
| Renal and urinary disorders | 3 (3.6) | 2 (2.4) |
| Cardiac disorders* | <3 | 3 (3.6) |

*formatted to protect treatment blind; Treatment-Emergent Serious Adverse Events By System Organ Class And Preferred Term (n ≥ 3 overall) (safety-evaluable population, n = 251)

TABLE 13
Selected adverse events

| | Crenezumab- | Placebo- |
|---|---|---|
| | Carrier | Carrier |
| | n = 84 | n = 84 |
| | n (%) | n (%) |
| Participants with any SAE | 84 (100) | 84 (100) |
| Total number of SAEs | 2164 | 2295 |
| ARIA-E | 0 | 0 |
| ARIA-H | 4 (4.8) | 2 (2.4) |
| Injection reactions* | 34 (40.5) | 31 (36.9) |
| Infusion-related reactions** | 25 (29.8) | 24 (28.6) |
| COVID-19 (confirmed) | 12 (14.3) | 2 (2.4) |

*AEs occurring during or within 24 hours of subcutaneous injection of study drug and assessed by the investigator as related to the injection; All injection reactions were non-serious, all were mild or moderate, none led to treatment withdrawal
**AEs occurring during or within 24 hours of intravenous infusion of study drug and assessed by the investigator as related to the infusion; All infusion-related reactions were non-serious, all but one were mild or moderate, none led to treatment withdrawal

In addition, the study demonstrated crenezumab PK results that are consistent with historical data for subcutaneous crenezumab and lower than expected exposure for IV crenezumab although limited by small sample size.

Additional exploratory outcome measures of interest included clinical indicators, fluid biomarkers, imaging biomarkers, and other outcome measures. Clinical indicators included changes from baseline over time in the following cognitive measures: Trail Making Test (Armitage, Psychological Monographs, 1946; 60: i-48), Mini-Mental State Exam (MMSE) (Folstein et al., J. Psychiatr. Res., 1975; 12:189-198), Repeatable Battery for the Assessment of Neuropsychological Status (RBANS), Index Scores (Randolph, Repeatable Battery for the Assessment of Neuropsychological Status. San Antonio, TX: The Psychological Corporation, 1998), scores of each of the components of the API ADAD Composite Cognitive Test Battery, Preclinical Alzheimer's Cognitive Composite (PACC) (Donohue et al., JAMA Neurology, 2014; 71:961-970), which for this trial will include the FCSRT free and cued recall, MMSE, RBANS story recall, and RBANS coding score. Additionally, clinical endpoints not examined in secondary outcome measures were explored and assessed: changes in the Neuropsychiatric Inventory (NPI) (Cummings et al., Neurology, 1994; 44:2308-2314 and Cummings, Neurology, 1997; 48: S10-S16) total score, items, and factors, changes in the Geriatric Depression Scale (GDS) (Sheikh and Yesavage, Clinical Gerontologist: J Aging Mental Health, 1986; 5:165-173) total score, changes in Functional Assessment Staging of Alzheimer's Disease (FAST) (Sclan and Reisberg, Int Psychogeriatr, 1992; 4:55-69) total score, and changes in Subjective Memory Checklist (Acosta-Baena et al., Lancet Neurol, 2011; 10:213-220). Additional outcomes measured include: changes in primary, secondary, and exploratory outcomes in mutation non-carriers treated with placebo; comparisons of clinical and biomarker outcomes between carriers treated with placebo and non-carriers; changes in primary, secondary, and exploratory outcomes in carriers and non-carriers as functions of APOE genotype and other genetic variations; short-term changes in imaging measures as functions of initiation (e.g., baseline to 12 weeks); and analyses of outcome measures in relation to one another and in relation to baseline characteristics.

### Example 3: Plasma Biomarker Findings from the Alzheimer's Prevention Initiative Autosomal Dominant Alzheimer's Disease Colombia Trial

This example describes the clinical and biomarker outcomes of participants enrolled in the study described in Example 2.

Plasma assays were performed on the Roche Elecsys platform using the Neurotoolkit assays and measured Aβ42, Aβ40, pTau181, pTau217, NfL, GFAP, YKL-40, and sTREM2.

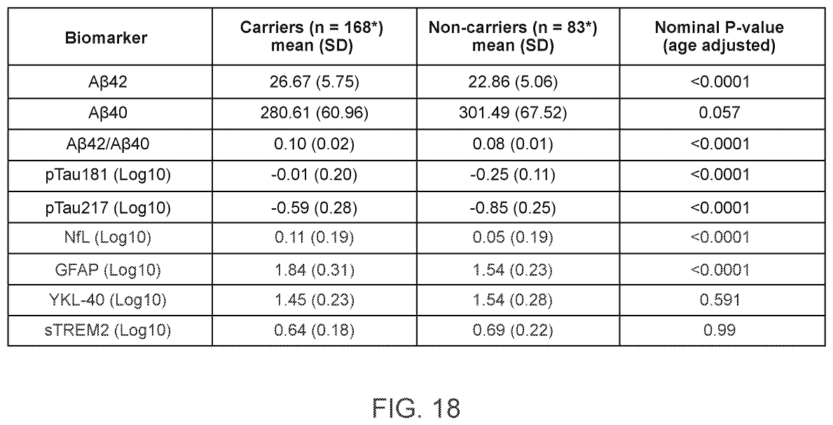

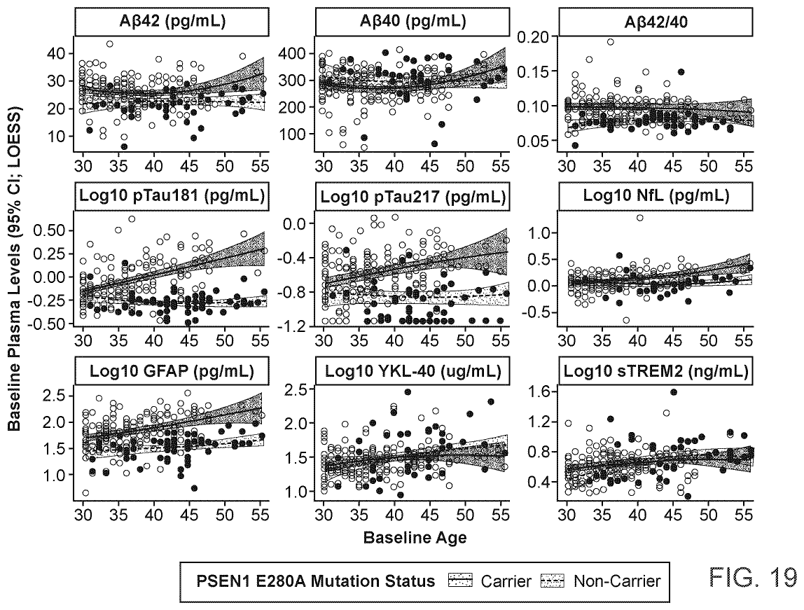

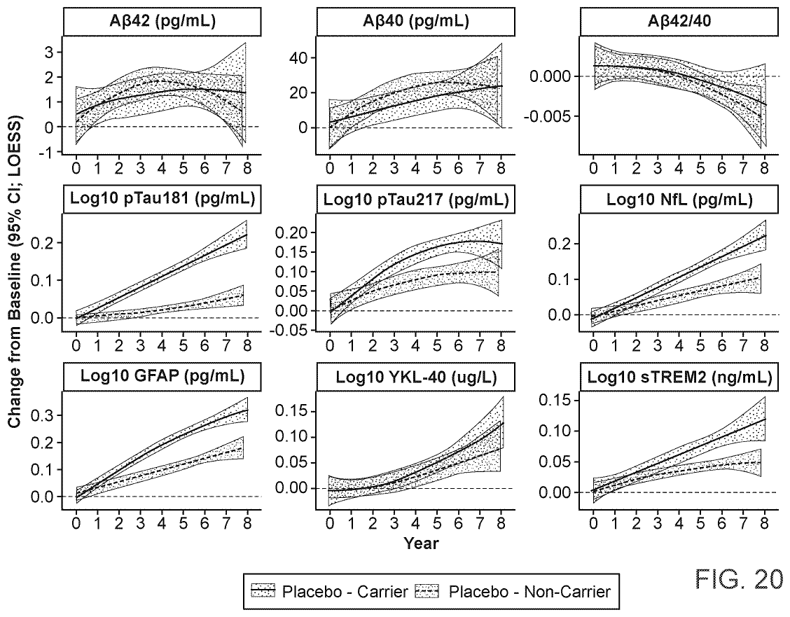

FIG. 18 shows the baseline plasma biomarker findings in carriers and non-carriers. As shown in FIG. 19, plasma Aβ42 concentrations and Aβ42/Aβ40 ratios are elevated in the mutation carrier. Baseline plasma Aβ42/Aβ40 elevations in untreated mutation carriers were unrelated to age or Aβ plaque burden. Further the Log10 plasma pTau181, pTau217, NfL, and GFAP concentrations are associated with age in the mutation group. Among non-carriers administered placebo and non-carrier, longitudinal plasma biomarker changes were observed. As shown in FIG. 20, there was a greater increase in Log10 plasma pTau181, pTau217, NfL, GFAP, and sTREM2 in the mutation carrier group.

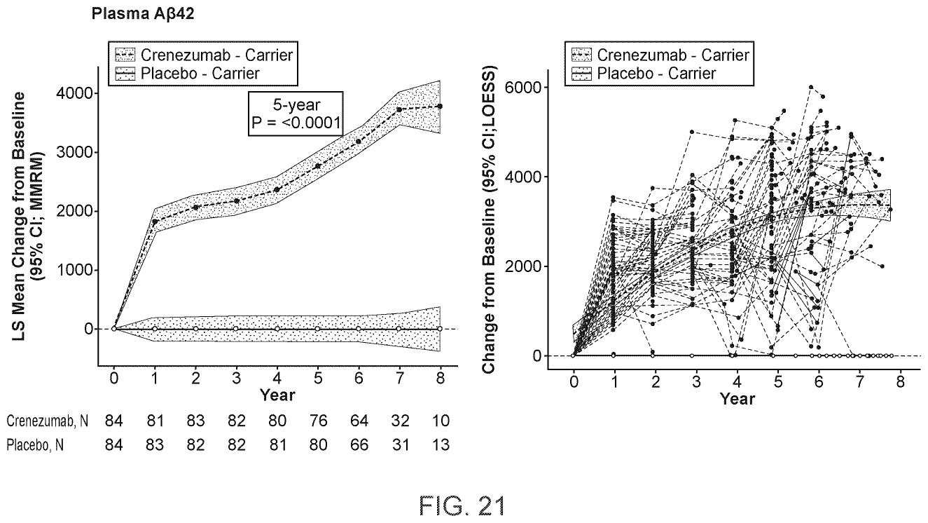

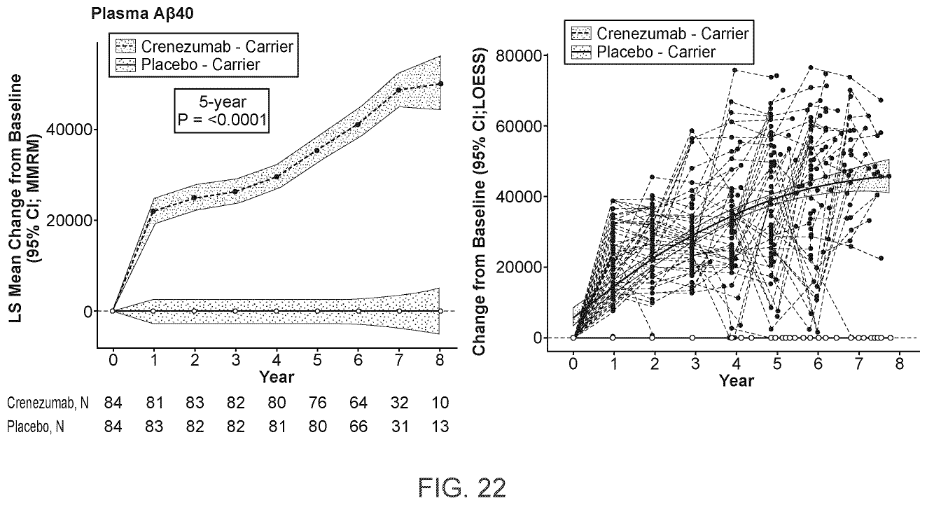

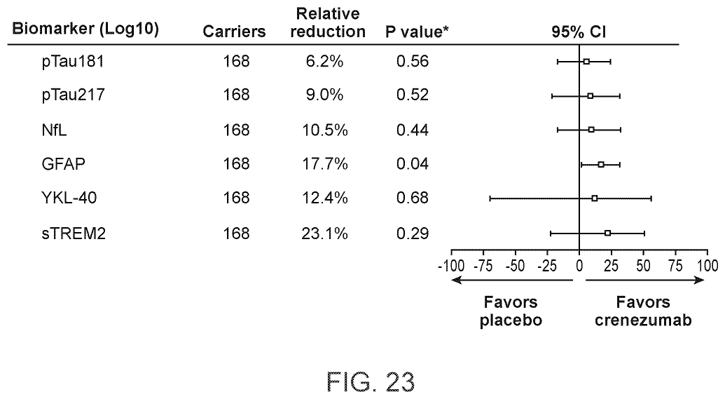

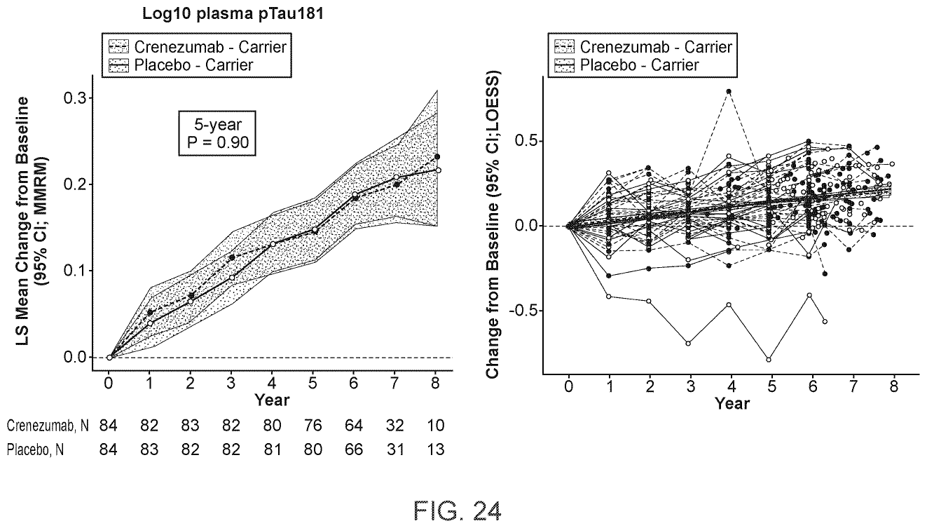

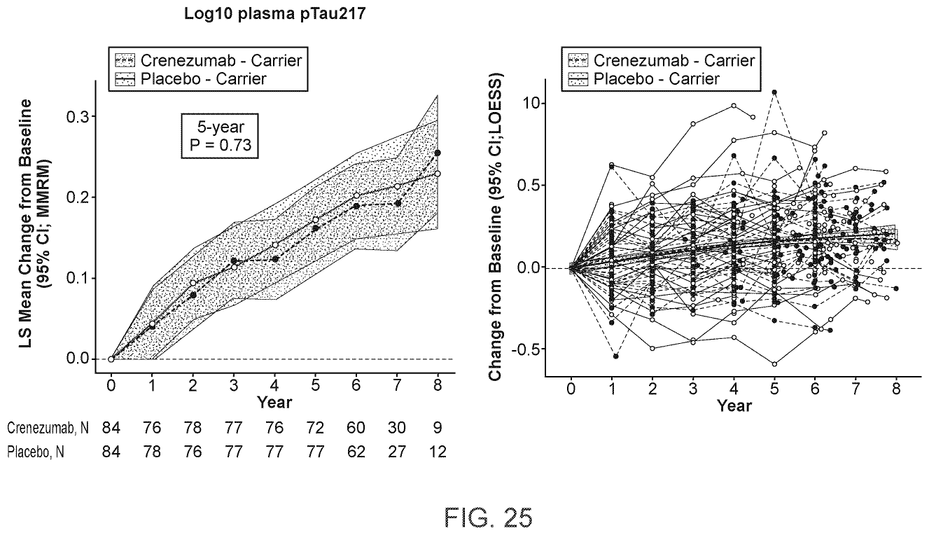

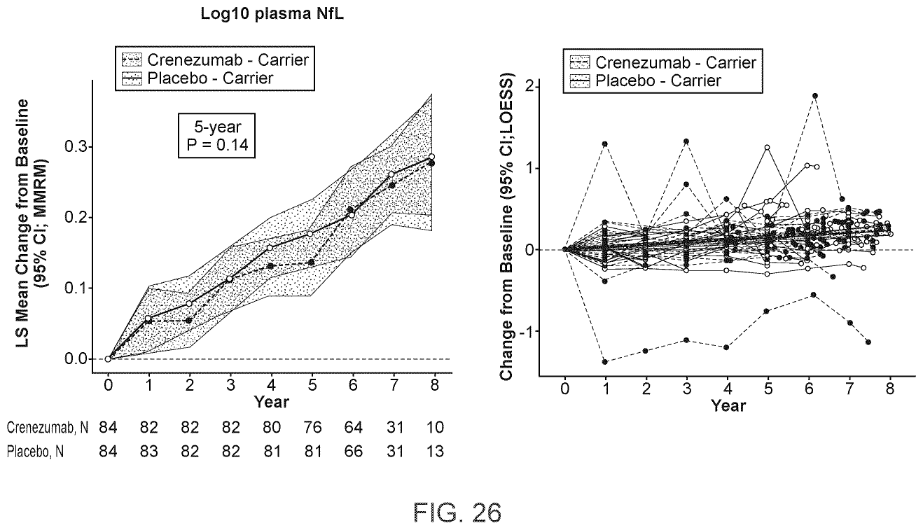

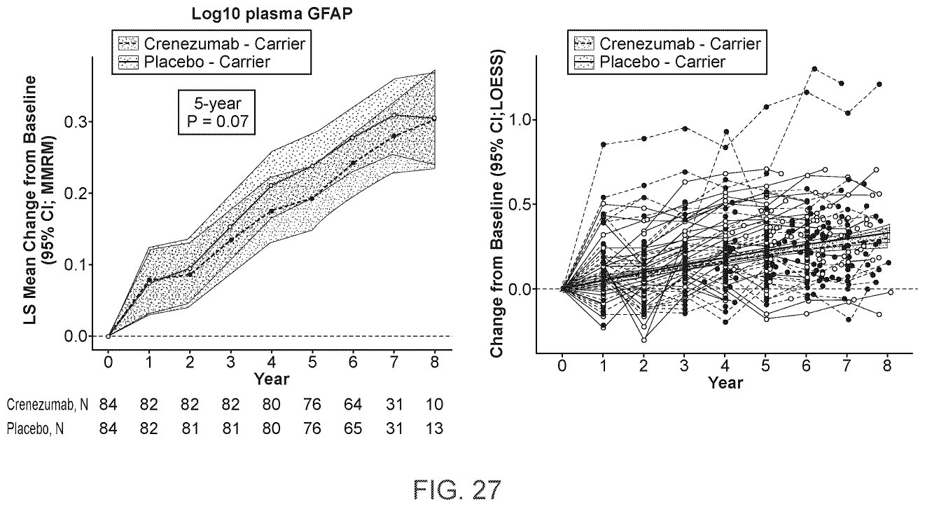

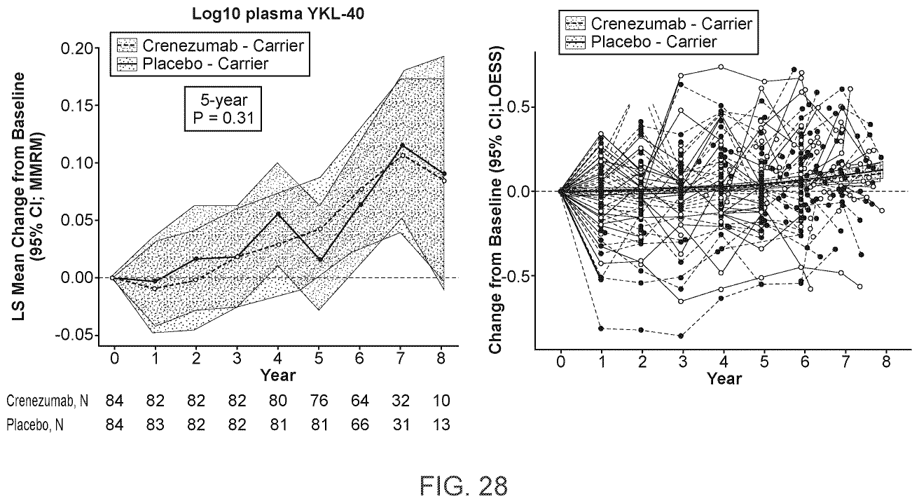

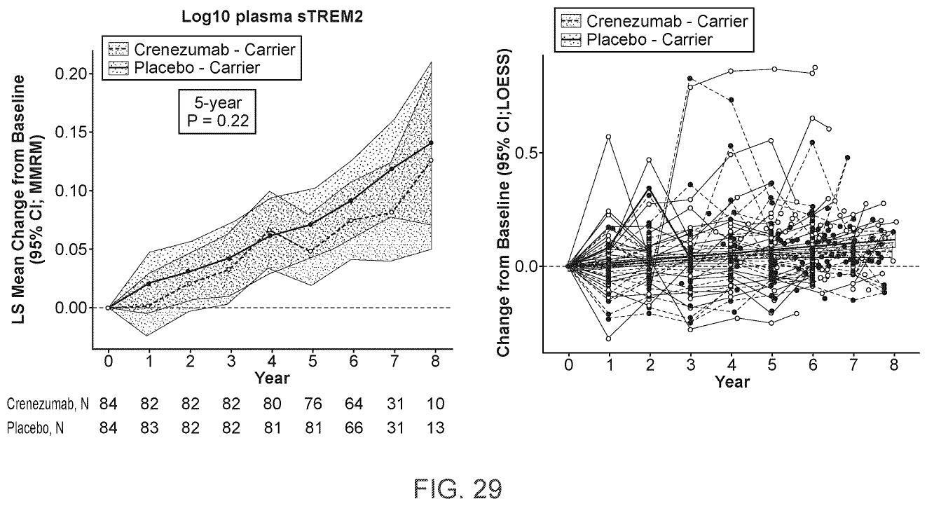

FIGS. 21-29 show plasma biomarker outcomes in the crenezumab and placebo treated carrier groups. As shown in FIGS. 21 and 22, plasma Aβ42 and Aβ40 increases were observed in the crenezumab-treated carrier group, as compared to placebo-treated carriers. In FIG. 23, the relative reduction in plasma Log10 pTau181, pTau217, NfL, GFAP, YKL-40, and sTREM2 in the crenezumab carrier group compared to that in the placebo carrier group is shown. Minimal but notable differences were observed in the plasma Log10 pTau181, pTau217, NfL, GFAP, YKL-40, and sTREM2 between placebo-treated carriers and crenezumab-treated carriers, with an increase in these biomarkers observed in both groups (FIGS. 24-29).

Although the foregoing invention has been described in some detail by way of illustration and example for purposes of clarity of understanding, the descriptions and examples should not be construed as limiting the scope of the invention. The disclosures of all patent applications and publications and scientific literature cited herein are expressly incorporated in their entirety by reference for any purpose.

### Sequence Listing Key

SEQUENCE LISTING KEY

| SEQ | |
|---|---|
| ID NO: | Sequence |
| 1 | Human Aβ1-42 amino acid sequence: |
| | DAEFRHDSGYEVHHQKLVFFAEDVGSNKGAIIGLMVGGVVIA |
| 2 | Crenezumab HVR-H1 amino acid sequence: GFTFSSYGMS |
| 3 | Crenezumab HVR-H2 amino acid sequence: |
| | SINSNGGSTYYPDSVK |
| 4 | Crenezumab HVR-H3 amino acid sequence: GDY |
| 5 | Crenezumab heavy chain amino acid sequence (HVR regions marked |
| | in bold text): |
| | EVQLVESGGGLVQPGGSLRLSCAASGFTFSSYGMSWVRQAP |
| | GKGLELVASINSNGGSTYYPDSVKGRFTISRDNAKNSLYLQM |
| | NSLRAEDTAVYYCASGDYWGQGTTVTVSSASTKGPSVFPLAP |
| | CSRSTSESTAALGCLVKDYFPEPVTVSWNSGALTSGVHTFPAV |
| | LQSSGLYSLSSVVTVPSSSLGTKTYTCNVDHKPSNTKVDKRVE |
| | SKYGPPCPPCPAPEFLGGPSVFLFPPKPKDTLMISRTPEVTCVV |
| | VDVSQEDPEVQFNWYVDGVEVHNAKTKPREEQFNSTYRVVS |
| | VLTVLHQDWLNGKEYKCKVSNKGLPSSIEKTISKAKGQPREPQ |
| | VYTLPPSQEEMTKNQVSLTCLVKGFYPSDIAVEWESNGQPEN |
| | NYKTTPPVLDSDGSFFLYSRLTVDKSRWQEGNVFSCSVMHEA |
| | LHNHYTQKSLSLSLG |
| 6 | Crenezumab HVR-L1 amino acid sequence: |
| | RSSQSLVYSNGDTYLH |
| 7 | Crenezumab HVR-L2 amino acid sequence: KVSNRFS |
| 8 | Crenezumab HVR-L3 amino acid sequence: SQSTHVPWT |
| 9 | Crenezumab light chain amino acid sequence (HVR regions marked |
| | in bold and underlined text): |
| | DIVMTQSPLSLPVTPGEPASISCRSSQSLVYSNGDTYLHWYLQ |
| | KPGQSPQLLIYKVSNRFSGVPDRFSGSGSGTDFTLKISRVEAED |
| | VGVYYCSQSTHVPWTFGQGTKVEIKRTVAAPSVFIFPPSDEQL |
| | KSGTASVVCLLNNFYPREAKVQWKVDNALQSGNSQESVTEQ |
| | DSKDSTYSLSSTLTLSKADYEKHKVYACEVTHQGLSSPVTKSF |
| | NRGEC |
| 10 | Variable region heavy chain sequence: |
| | EVQLVESGGGLVQPGGSLRLSCAASGFTFSSYGMSWVRQAPG |
| | KGLELVASINSNGGSTYYPDSVKGRFTISRDNAKNSLYLQMNS |
| | LRAEDTAVYYCASGDYWGQGTTVTVSS |
| 11 | Variable region light chain sequence: |
| | DIVMTQSPLSLPVTPGEPASISCRSSQSLVYSNGDTYLHWYLQ |
| | KPGQSPQLLIYKVSNRFSGVPDRFSGSGSGTDFTLKISRVEAED |
| | VGVYYCSQSTHVPWTFGQGTKVEIK |

### Enumerated Embodiments

Embodiment 1. A method of delaying onset of at least one symptom in a human patient with a genetic mutation that causes familial Alzheimer's Disease (AD) comprising administering to the human patient an effective amount of a humanized monoclonal anti-amyloid beta (Aβ) antibody,
wherein administering such treatment to a plurality of human patients results in a delayed onset of at least one symptom in the plurality of human patients compared to a reference onset of at least one symptom,
wherein the reference onset of at least one symptom is of a plurality of human patients who have received a placebo,
wherein the antibody comprises six hypervariable regions (HVRs), wherein
(i) HVR-H1 comprises the amino acid sequence set forth in SEQ ID NO:2;
(ii) HVR-H2 comprises the amino acid sequence set forth in SEQ ID NO:3;
(iii) HVR-H3 comprises the amino acid sequence set forth in SEQ ID NO:4;
(iv) HVR-L1 comprises the amino acid sequence set forth in SEQ ID NO:6;
(v) HVR-L2 comprises the amino acid sequence set forth in SEQ ID NO:7; and
(vi) HVR-L3 comprises the amino acid sequence set forth in SEQ ID NO:8.

Embodiment 2. A method of slowing cognitive decline in a human patient with a genetic mutation that causes familial Alzheimer's Disease (AD) comprising administering to the human patient an effective amount of a humanized monoclonal anti-amyloid beta (Aβ) antibody,
wherein administering such treatment to a plurality of human patients results in a delayed cognitive decline in the plurality of human patients compared to a reference cognitive decline,
wherein the reference cognitive decline is of a plurality of human patients who have received a placebo,
wherein the antibody comprises six hypervariable regions (HVRs), wherein
(i) HVR-H1 comprises the amino acid sequence set forth in SEQ ID NO:2;
(ii) HVR-H2 comprises the amino acid sequence set forth in SEQ ID NO:3;
(iii) HVR-H3 comprises the amino acid sequence set forth in SEQ ID NO:4;
(iv) HVR-L1 comprises the amino acid sequence set forth in SEQ ID NO:6;
(v) HVR-L2 comprises the amino acid sequence set forth in SEQ ID NO:7; and
(vi) HVR-L3 comprises the amino acid sequence set forth in SEQ ID NO:8.

Embodiment 3. A method of preventing cognitive impairment in a human patient with a genetic mutation that causes familial Alzheimer's Disease (AD) comprising administering to the human patient an effective amount of a humanized monoclonal anti-amyloid beta (Aβ) antibody,
wherein administering such treatment to a plurality of human patients results in a reduced cognitive impairment in the plurality of human patients compared to a reference cognitive impairment,
wherein the reference cognitive impairment is of a plurality of human patients who have received a placebo,
wherein the antibody comprises six hypervariable regions (HVRs), wherein
(i) HVR-H1 comprises the amino acid sequence set forth in SEQ ID NO:2;
(ii) HVR-H2 comprises the amino acid sequence set forth in SEQ ID NO:3;
(iii) HVR-H3 comprises the amino acid sequence set forth in SEQ ID NO:4;
(iv) HVR-L1 comprises the amino acid sequence set forth in SEQ ID NO:6;
(v) HVR-L2 comprises the amino acid sequence set forth in SEQ ID NO:7; and
(vi) HVR-L3 comprises the amino acid sequence set forth in SEQ ID NO:8.

Embodiment 4. The method of embodiment 1, wherein administering such treatment results in a delay in onset of at least one symptom compared to the reference onset of at least one symptom after treatment of about five years or longer.

Embodiment 5. The method of embodiment 2, wherein administering such treatment results in a slowing of cognitive decline in the plurality of human patients compared to the reference cognitive decline after treatment of about five years or longer.

Embodiment 6. The method of embodiment 3, wherein administering such treatment results in a prevention or reduction of cognitive impairment in the plurality of human patients compared to the reference cognitive impairment after treatment of about five years or longer.

Embodiment 7. The method of any one of embodiments 1-6,
wherein the delay in onset of at least one symptom, the slowing in cognitive decline, or the prevention of cognitive impairment is measured using an API ADAD Cognitive Composite Test Battery,
wherein, in the API ADAD Cognitive Composite Test Battery, administering such treatment to the plurality of human patients results in a reduced annualized rate of change on an API ADAD Composite score of the plurality of human patients relative to a reference annualized rate of change on an API ADAD Composite score, and
wherein the reference annualized rate of change on an API ADAD Composite score is an annualized rate of change on an API ADAD Composite score of a plurality of human patients who have received the placebo.

Embodiment 8. The method of embodiment 7, wherein the API ADAD Composite Cognitive Test Battery comprises a Word List Recall, Multilingual Naming Test, Mini-Mental State Examination (MMSE), CERAD Constructional praxis, and Raven's Progressive Matrices.

Embodiment 9. The method of any one of embodiments 7-8, wherein administering such treatment results in a reduced annualized rate of change in the API ADAD Composite score after treatment of about five years or longer.

Embodiment 10. The method of any one of embodiments 7-9, wherein administering such treatment results in a reduction of the annualized rate of change on the API ADAD Composite score in the plurality of human patients by at least 20% relative to the reference annualized rate of change on the API ADAD Composite score.

Embodiment 11. The method of any one of embodiments 7-9, wherein administering such treatment results in a reduction of the annualized rate of change in the API ADAD Composite score in the plurality of human patients by at least 30% relative to the reference annualized rate of change on the API ADAD Composite score.

Embodiment 12. The method of any one of embodiments 7-9, wherein administering such treatment results in a reduction of the annualized rate of change in the API ADAD Composite score in the plurality of human patients by 20% to 40% relative to the reference annualized rate of change on the API ADAD Composite score.

Embodiment 13. The method of any one of embodiments 1-12, wherein administering such treatment to the plurality of human patients results in a reduced annualized rate of change on a Free and Cued Selective Reminding Task (FCSRT) Cueing Index of the plurality of human patients relative to a reference annualized rate of change on a FCSRT Cueing Index,
wherein the reference annualized rate of change on the FCSRT Cueing Index is an annualized rate of change on a FCSRT Cueing Index of a plurality of human patients who have received the placebo.

Embodiment 14. The method of embodiment 13, wherein administering such treatment results in a reduced annualized rate of change on the FCSRT Cueing Index in the plurality of human patients compared to the reference annualized rate of change on the FCSRT Cueing Index after treatment of about five years or longer.

Embodiment 15. The method of any one of embodiments 13-14, wherein administering such treatment results in a reduction of the annualized rate of change of the FCSRT Cueing Index in the plurality of human patients by at least 10% relative to the reference annualized rate of change on the FCSRT Cueing Index.

Embodiment 16. The method of any one of embodiment 13-14, wherein administering such treatment results in a reduction of the annualized rate of change of the FCSRT Cueing Index in the plurality of human patients by at least 20% relative to the reference annualized rate of change on the FCSRT Cueing Index.

Embodiment 17. The method of any one of embodiments 13-14, wherein administering such treatment results in a reduction of the annualized rate of change of the FCSRT Cueing Index in the plurality of human patients by 10% to about 30% relative to the reference annualized rate of change on the FCSRT Cueing Index.

Embodiment 18. The method of any one of embodiments 13-17, wherein the FCSRT Cueing Index is assessed using controlled learning.

Embodiment 19. The method of any one of embodiments 1-18, wherein administering such treatment to the plurality of human patients results in an increased time to progression from preclinical AD to mild cognitive impairment due to AD or from preclinical AD to dementia due to AD in the plurality of human patients relative to a reference time to progression from preclinical AD to mild cognitive impairment due to AD or from preclinical AD to dementia due to AD,
wherein the reference time to progression from preclinical AD to mild cognitive impairment due to AD or from preclinical AD to dementia due to AD is a time to progression of a plurality of human patients who have received the placebo.

Embodiment 20. The method of embodiment 19, wherein administering such treatment results in an increased time to progression from preclinical AD to mild cognitive impairment due to AD or from preclinical AD to dementia due to AD in the plurality of human patients relative to the reference time to progression from preclinical AD to mild cognitive impairment due to AD or from preclinical AD to dementia due to AD after treatment of about five years or longer.

Embodiment 21. The method of any one of embodiments 19-20, wherein administering such treatment results in an increase of the time to progression from preclinical AD to mild cognitive impairment due to AD or from preclinical AD to dementia due to AD in the plurality of human patients by at least 10% as compared to a reference time to progression from preclinical AD to mild cognitive impairment due to AD or from preclinical AD to dementia due to AD.

Embodiment 22. The method of any one of embodiments 19-20, wherein administering such treatment results in an increase of the time to progression from preclinical AD to mild cognitive impairment due to AD or from preclinical AD to dementia due to AD in the plurality of human patients by at least 20% relative to a reference time to progression from preclinical AD to mild cognitive impairment due to AD or from preclinical AD to dementia due to AD.

Embodiment 23. The method of any one of embodiments 19-20, wherein administering such treatment results in an increase of the time to progression from preclinical AD to mild cognitive impairment due to AD or from preclinical AD to dementia due to AD in the plurality of human patients by 10% to 30% relative to a reference time to progression from preclinical AD to mild cognitive impairment due to AD or from preclinical AD to dementia due to AD.

Embodiment 24. The method of any one of embodiments 1-23, wherein administering such treatment to the plurality of human patients results in an increased time to progression to non-zero in the Clinical Dementia Rating (CDR) Scale global score of the plurality of human patients relative to a reference time to progression to non-zero in the CDR Scale global score, wherein the reference time to progression to non-zero in the CDR Scale global score is a time to progression of a plurality of human patients who have received the placebo.

Embodiment 25. The method of embodiment 24, wherein the CDR Scale global score describes impairment in memory, orientation, judgment and problem solving, community affairs, home and hobbies, and personal care.

Embodiment 26. The method of any one of embodiments 24-25, wherein administering such treatment results in an increased time to progression to non-zero in the CDR Scale global score in the plurality of human patients by 5% relative to a reference time to progression to progression to non-zero in the CDR Scale global score.

Embodiment 27. The method of any one of embodiments 24-25, wherein administering such treatment results in an increased time to progression to non-zero in the CDR Scale global score in the plurality of human patients by 10% relative to a reference time to progression to progression to non-zero in the CDR Scale global score.

Embodiment 28. The method of any one of embodiments 24-25, wherein administering such treatment results in an increased time to progression to non-zero in the CDR Scale global score in the plurality of human patients by 5% to 20% relative to a reference time to progression to progression to non-zero in the CDR Scale global score.

Embodiment 29. The method of any one of embodiments 1-28, wherein administering such treatment to the plurality of human patients results in a reduced annualized rate of change on a Clinical Dementia Rating (CDR) Scale Sum of Boxes of the plurality of human patients relative to a reference annualized rate of change on a CDR Scale Sum of Boxes,
wherein the reference annualized rate of change on a CDR Scale Sum of Boxes is an annualized rate of change on a CDR Scale Sum of Boxes of a plurality of human patients who have received placebo.

Embodiment 30. The method of embodiment 29, wherein administering such treatment results in a reduced annualized rate of change on a CDR Scale Sum of Boxes of the plurality of human patients compared to the reference annualized rate of change on a CDR Scale Sum of Boxes after treatment of about five years or longer.

Embodiment 31. The method of any one of embodiments 29-30, wherein administering such treatment results in a reduced annualized rate of change in a CDR Scale Sum of Boxes global score in the plurality of human patients by at least 5% relative to a reference CDR Scale Sum of Boxes global score.

Embodiment 32. The method of any one of embodiments 29-30, wherein administering such treatment results in a reduced annualized rate of change in a CDR Scale Sum of Boxes global score in the plurality of human patients by at least 10% relative to a reference CDR Scale Sum of Boxes global score.

Embodiment 33. The method of any one of embodiments 29-30 wherein administering such treatment results in a reduced annualized rate of change in a CDR Scale Sum of Boxes global score in the plurality of human patients by 5% to 20% relative to a reference CDR Scale Sum of Boxes global score.

Embodiment 34. The method of any one of embodiments 1-33, wherein administering such treatment to the plurality of human patients results in a reduced annualized rate of change in a measure of overall neurocognitive functioning of the plurality of human patients relative to a reference annualized rate of change in a measure of overall neurocognitive functioning
wherein the reference annualized rate of change in a measure of overall neurocognitive functioning is an annualized rate of change in a measure of overall neurocognitive functioning of the plurality of human patients who have received placebo.

Embodiment 35. The method of embodiment 34, wherein the annualized rate of change in a measure of overall neurocognitive functioning is determined using a Repeatable Battery for the Assessment of Neuropsychological Status (RBANS) score.

Embodiment 36. The method of embodiment 35, wherein administering such treatment results in a reduction of an annualized rate of change of a RBANS score in the plurality of human patients compared to a reference annualized rate of change of a RBANS score, wherein the reference annualized rate of change of a RBANS score is an annualized rate of change of a RBANS score of a plurality of human patients who have received placebo.

Embodiment 37. The method of any one of embodiments 35-36, wherein administering such treatment results in a reduction of the annualized rate of change of a RBANS score in the plurality of human patients compared to the reference annualized rate of change of a RBANS score after treatment of about 5 years or longer.

Embodiment 38. The method of any one of embodiments 35-37, wherein administering such treatment results in a reduction of the RBANS score by at least 4% relative to the reference RBANS score.

Embodiment 39. The method of any one of embodiments 35-37, wherein administering such treatment results in a reduction of the RBANS score by at least 5% relative to the reference RBANS score.

Embodiment 40. The method of any one of embodiments 35-37, wherein administering such treatment results in a reduction of the RBANS score by 30% to 60% relative to the reference RBANS score.

Embodiment 41. The method of any one of embodiments 1-40, wherein administering such treatment to the plurality of human patients results in an effect on a tau-based CSF biomarker compared to a reference tau-based CSF biomarker,
wherein the reference tau-based CSF biomarker is the tau-based CSF biomarker of a plurality of human patients who have received the placebo.

Embodiment 42. The method of embodiment 41, wherein the tau-based CSF biomarker is measured using positron emission tomography.

Embodiment 43. The method of any one of embodiments 41-42, wherein administering such treatment results in a reduction of annualized rate of change in the tau-based CSF biomarker in the plurality of human patients compared to a reference annualized rate of change in the tau-based CSF biomarker, wherein the reference tau-based CSF biomarker is an annualized rate of change in the tau-based CSF biomarker of a plurality of human patients who have received the placebo.

Embodiment 44. The method of embodiment 43, wherein administering such treatment results in a reduction of annualized rate of the tau-based CSF biomarker in the plurality of human patients by at least 30% relative to the reference tau-based CSF biomarker, said tau-based CSF biomarker being a phospho-tau [ptau]-based CSF biomarker.

Embodiment 45. The method of embodiment 43, wherein administering such treatment results in a reduction of annualized rate of the tau-based CSF biomarker in the plurality of human patients by 30%-50% relative to the reference tau-based CSF biomarker, said tau-based CSF biomarker being a phospho-tau [ptau]-based CSF biomarker.

Embodiment 46. The method of embodiment 43, wherein administering such treatment results in a reduction of annualized rate of the tau-based CSF biomarker in the plurality of human patients by at least 20% relative to the reference tau-based CSF biomarker, said tau-based CSF biomarker being a total-tau [ttau]-based CSF biomarker.

Embodiment 47. The method of embodiment 43, wherein administering such treatment results in a reduction of annualized rate of the tau-based CSF biomarker in the plurality of human patients by 20% to 40% relative to the reference tau-based CSF biomarker, said tau-based CSF biomarker being a total-tau [ttau]-based CSF biomarker.

Embodiment 48. The method of any one of embodiments 1-47, wherein administering such treatment to the plurality of human patients results in an effect on a brain tau load compared to a reference a brain tau load,
wherein the reference brain tau load is a brain tau load of a plurality of human patients who have received the placebo.

Embodiment 49. The method of embodiment 48, wherein the brain tau load is measured using positron emission tomography (tau-PET).

Embodiment 50. The method of embodiment 49, wherein administering such treatment results in a reduction of annualized rate of change in a tau-PET measurement in the plurality of human patients relative to a reference annualized rate of change in a tau-PET measurement, wherein the reference annualized rate of change in a tau-PET measurement is an annualized rate of change in the tau-PET measurement of a plurality of human patients who have received the placebo.

Embodiment 51. The method of embodiment 50, wherein administering such treatment results in a reduced Standardized Uptake Value Ratio (SUVR) of entorhinal cortex (ERC) tau-PET measurement in the plurality of human patients as compared to a reference SUVR of ERC tau-PET, wherein the reference SUVR of aERC tau-PET is a SUVR of ERC tau-PET of the plurality of human patients who have received the placebo.

Embodiment 52. The method of embodiment 51, wherein the tau-PET is measured using Tau Probe 1, which is [18F] GTP1.

Embodiment 53. The method of any one of embodiments 50-52, wherein administering such treatment results in a reduction of annualized rate of the tau-PET in the plurality of human patients by at least 50% relative to the reference tau-PET.

Embodiment 54. The method of any one of embodiments 50-52, wherein administering such treatment results in a reduction of annualized rate of the tau-PET in the plurality of human patients by 40% to 60% relative to the reference tau-PET.

Embodiment 55. The method of any one of embodiments 1-54, wherein administering such treatment to the plurality of human patients results in a reduction of cerebral fibrillary amyloid burden in a predefined region of interest of the plurality of human patients relative to a reference cerebral fibrillary amyloid burden in a predefined region of interest,
wherein the reference cerebral fibrillary amyloid burden in a predefined region of interest is a cerebral fibrillary amyloid burden in a predefined region of interest of a plurality of human patients who have received the placebo.

Embodiment 56. The method of embodiment 55, wherein the cerebral fibrillary amyloid burden is measured using florbetapir positron emission tomography (PET).

Embodiment 57. The method of any one of embodiments 55-56, wherein administering such treatment results in a reduction of annualized rate of change in amyloid burden in the plurality of human patients compared to a reference annualized rate of change in amyloid burden, wherein the reference annualized rate of change in amyloid burden is an annualized rate of change in amyloid burden of a plurality of human patients who have received the placebo.

Embodiment 58. The method of embodiment 57, wherein administering such treatment results in a reduction of annualized rate of change in amyloid burden measured by PET in the plurality of human patients by 3% relative to a reference amyloid burden measured by PET.

Embodiment 59. The method of embodiment 57, wherein administering such treatment results in a reduction of annualized rate of change in amyloid burden measured by PET in the plurality of human patients by 3% to 10% relative to a reference amyloid burden measured by PET.

Embodiment 60. The method of any one of embodiments 1-59, wherein administering such treatment to the plurality of human patients results in a reduced decline in regional cerebral metabolic rate of glucose (CMRgI) of the plurality of human patients relative to a reference CMRgI,
wherein the reference CMRgI is a CMRgI of the plurality of human patients who have received the placebo.

Embodiment 61. The method of embodiment 60, wherein the CMRgI is measured using FDG (fluorodeoxyglucose)-positron emission tomography (PET).

Embodiment 62. The method of embodiment 61, wherein administering such treatment results in a reduced FDG PET measurement in the plurality of human patients relative to a reference FDG PET measurement, wherein the reference FDG PET measurement is a FDG PET measurement of the plurality of human patients who have received the placebo.

Embodiment 63. The method of embodiment 62, wherein administering such treatment results in a reduced annualized Standardized Uptake Value Ratio (SUVR) of FDG PET measurement in the plurality of human patients as compared to a reference annualized SUVR of FDG PET, wherein the reference annualized SUVR of FDG PET is an annualized SUVR of FDG PET of the plurality of human patients who have received the placebo.

Embodiment 64. The method of embodiment 60-63, wherein administering such treatment results in a reduced decline in regional CMRgI of the plurality of human patients compared to the reference CMRgI after treatment of about five years or longer.

Embodiment 65. The method of any one of embodiments 63-64, wherein administering such treatment results in a reduced annualized Standardized Uptake Value Ratio (SUVR) of FDG PET measurement in the plurality of human patients by at least 10% as compared to a reference annualized SUVR of FDG PET.

Embodiment 66. The method of any one of embodiments 63-64, wherein administering such treatment results in a reduced annualized Standardized Uptake Value Ratio (SUVR) of FDG PET measurement in the plurality of human patients by 10% to 30% relative to a reference annualized SUVR of FDG PET.

Embodiment 67. The method of any one of embodiments 1-66, wherein administering such treatment to the plurality of human patients results in a reduced brain atrophy of the plurality of human patients relative to a reference brain atrophy,
wherein the reference brain atrophy is a brain atrophy of the plurality of human patients who have received the placebo.

Embodiment 68. The method of embodiment 67, wherein administering such treatment results in a reduction of the brain atrophy of the plurality of human patients compared to the reference brain atrophy after treatment of about five years or longer.

Embodiment 69. The method of any one of embodiments 1-68, wherein administering such treatment to the plurality of human patients results in a reduced annualized rate of change in the brain atrophy of the plurality of human patients relative to a reference annualized rate of change in the brain atrophy,
wherein the reference annualized rate of change in the brain atrophy is an annualized rate of change in the brain atrophy of the plurality of human patients who have received the placebo.

Embodiment 70. The method of embodiment 69, wherein administering such treatment results in a reduction of an annualized rate of change in the brain atrophy of the plurality of human patients compared to the reference annualized rate of change in the brain atrophy after treatment of about five years or longer.

Embodiment 71. The method of any one of embodiments 67-70, wherein the brain atrophy is measured using volumetric MRI.

Embodiment 72. The method of embodiment 71, wherein the volumetric MRI is measured in a whole brain.

Embodiment 73. The method of embodiment 72, wherein administering such treatment results in a reduction of an annualized rate of change in the brain atrophy of the plurality of human patients by at least 5% relative to the reference brain atrophy, said reduction being measured by a volumetric MRI on a whole brain.

Embodiment 74. The method of embodiment 72, wherein administering such treatment results in a reduction of an annualized rate of change in the brain atrophy of the plurality of human patients by 5% to 20% relative to the reference brain atrophy, said reduction being measured by a volumetric MRI on a whole brain.

Embodiment 75. The method of embodiment 71, wherein the volumetric MRI is measured in a bilateral hippocampus.

Embodiment 76. The method of embodiment 75, wherein administering such treatment results in a reduction of an annualized rate of change in the brain atrophy of the plurality of human patients by at least 1% relative to the reference brain atrophy, said reduction being measured by a volumetric MRI on a bilateral hippocampus.

Embodiment 77. The method of embodiment 75, wherein administering such treatment results in a reduction of an annualized rate of change in the brain atrophy of the plurality of human patients by 1% to 10% relative to the reference brain atrophy, said reduction being measured by a volumetric MRI on a bilateral hippocampus.

Embodiment 78. The method of any one of embodiments 1-77, wherein administering such treatment results in a reduction in change over baseline in a cognitive measurement of the plurality of human patients compared to a reference cognitive measurement,
wherein the reference cognitive measurement is a cognitive measurement of a plurality of human patients who have received the placebo,
wherein the cognitive measurement is selected from the group consisting of i) Trial Making Test, ii) Mini-Mental State Examination (MMSE), iii) Repeatable Battery for the Assessment of Neuropsychological Status (RBANS) Index Scores, iv) scores of each of the components of the API ADAD Composite Cognitive Test Battery, v) Preclinical Alzheimer's Cognitive Composite (PACC), and vi) other clinical endpoints.

Embodiment 79. The method of embodiment 78, wherein administering such treatment results in a reduction in change over baseline in the cognitive measurement of the plurality of human patients compared to the reference cognitive measurement after treatment of about five years or longer.

Embodiment 80. The method of any one of embodiments 1-79, wherein administering such treatment results in a reduction in change over baseline in a Neuropsychiatric Inventory (NPI) of the plurality of human patients compared to a reference NPI,
wherein the reference NPI is a NPI of a plurality of human patients who have received the placebo.

Embodiment 81. The method of embodiment 80, wherein administering such treatment results in a reduction in change over baseline in NPI after treatment of about five years or longer.

Embodiment 82. The method of any one of embodiments 1-81, wherein administering such treatment results in a reduction in change over baseline in a Geriatric Depression Scale (GDS) of the plurality of human patients compared to a reference GDS,
wherein the reference GDS is a GDS of a plurality of human patients who have received the placebo.

Embodiment 83. The method of embodiment 82, wherein administering such treatment results in a reduction in change over baseline in GDS after treatment of about five years or longer.

Embodiment 84. The method of any one of embodiments 1-83, wherein administering such treatment results in a reduction in change over baseline in a Changes in Functional Assessment Staging of Alzheimer's Disease (FAST) total score of the plurality of human patients compared to a reference FAST total score,
wherein the reference FAST total score is the FAST total score of a plurality of human patients who have received the placebo.

Embodiment 85. The method of embodiment 84, wherein administering such treatment results in a reduction in change over baseline in FAST total score after treatment of about five years or longer.

Embodiment 86. The method of any one of embodiments 1-85, wherein administering such treatment results in a reduction in change over baseline in a Changes in Subject Memory Checklist of the plurality of human patients compared to a reference Changes in Subject Memory Checklist,
wherein the reference Changes in Subject Memory Checklist is a Changes in Subject Memory Checklist of a plurality of human patients who have received the placebo.

Embodiment 87. The method of embodiment 86, wherein administering such treatment results in a reduction in change over baseline in Changes in Subject Memory Checklist score after treatment of about five years or longer.

Embodiment 88. The method of any one of embodiments 1-87, wherein the genetic mutation that causes familial AD is an autosomal dominant mutation causing Autosomal Dominant Alzheimer's Disease (ADAD).

Embodiment 89. The method of claim embodiment 88, wherein the ADAD comprises one or more mutations in one or more genes selected from the group consisting of presenilin 1 (PSEN1), presenilin 2 (PSEN2), and/or amyloid precursor protein (APP).

Embodiment 90. The method of any one of embodiments 1-89, wherein the humanized monoclonal anti-amyloid beta (Aβ) antibody is delivered intravenously.

Embodiment 91. The method of embodiment 90, wherein the humanized monoclonal anti-amyloid beta (Aβ) antibody is administered at a) a dose of about 60 mg/kg or higher; or b) a fixed dose of 4200 mg or higher; or c) a fixed dose of about 4200 mg.

Embodiment 92. The method of any one of embodiments 90-91, wherein the humanized monoclonal anti-amyloid beta (Aβ) antibody is delivered about every four weeks (Q4W).

Embodiment 93. The method of any one of embodiments 90-91, wherein the humanized monoclonal anti-amyloid beta (Aβ) antibody is delivered about Q4W for about 5 years.

Embodiment 94. The method of any one of embodiments 1-93, wherein the humanized monoclonal anti-amyloid beta (Aβ) antibody is delivered subcutaneously.

Embodiment 95. The method of embodiment 94, wherein the humanized monoclonal anti-amyloid beta (Aβ) antibody is delivered at a dose of about 720 mg or higher.

Embodiment 96. The method of embodiment 94, wherein the humanized monoclonal anti-amyloid beta (Aβ) antibody is delivered at a dose of about 300 mg.

Embodiment 97. The method of any one of embodiments 94-96, wherein, the humanized monoclonal anti-amyloid beta (Aβ) antibody is delivered every other week (Q2W).

Embodiment 98. The method of any one of embodiments 94-97, wherein the humanized monoclonal anti-amyloid beta (Aβ) antibody is delivered about Q2W for about 5 years.

Embodiment 99. The method of any one of embodiments 1-98, wherein the humanized monoclonal anti-amyloid beta (Aβ) antibody comprises a heavy chain variable region (VH) comprising the amino acid sequence of SEQ ID NO: 10 and a light chain variable region (VL) comprising the amino acid sequence of SEQ ID NO:11.

Embodiment 100. The method of any one of embodiments 1-99, wherein the humanized monoclonal anti-amyloid beta (Aβ) antibody comprises a heavy chain comprising the amino acid sequence of SEQ ID NO:5 and a light chain comprising the amino acid sequence of SEQ ID NO: 9.

Embodiment 101. The method of any one of embodiments 1-100, wherein administering such treatment results in a reduced Standardized Uptake Value Ratio (SUVR) of amyloid PET measurement in the plurality of human patients as compared to a reference SUVR of amyloid PET, wherein the reference SUVR of amyloid PET is a SUVR of amyloid PET of the plurality of human patients who have received the placebo.

Embodiment 102. The method of embodiment 101, wherein administering such treatment results in a reduced Standardized Uptake Value Ratio (SUVR) of amyloid PET measurement in the plurality of human patients by at least 3% as compared to the reference SUVR of amyloid PET.

Embodiment 103. The method of any one of embodiments 101-102, wherein administering such treatment results in a reduced Standardized Uptake Value Ratio (SUVR) of amyloid PET measurement in the plurality of human patients by at least 10% relative to the reference SUVR of amyloid PET.

Embodiment 104. The method of any one of embodiments 101-102, wherein administering such treatment results in a reduced Standardized Uptake Value Ratio (SUVR) of amyloid PET measurement in the plurality of human patients by 3% to 10% relative to the reference SUVR of amyloid PET.

Embodiment 105. The method of any one of embodiments 102-104, wherein administering such treatment results in a reduction of cerebrospinal fluid (CSF) neurofilament light (CSF NfL) in the plurality of human patients as compared to a reference CSF NfL, wherein the reference CSF NfL is from the plurality of human patients who have received the placebo.

Embodiment 106. The method of embodiment 105, wherein administering such treatment results in a relative reduction of CSF NfL in the plurality of human patients of at least 10% relative to the reference CSF NfL.

Embodiment 107. The method of embodiment 105, wherein administering such treatment results in a relative reduction of CSF NfL in the plurality of human patients of at least 20% relative to the reference CSF NfL.

Embodiment 108. The method of embodiment 105, wherein administering such treatment results in a relative reduction of CSF NfL in the plurality of human patients of 10% to 30% relative to the reference CSF NfL.

Embodiment 109. The method of any one of embodiments 1-108, wherein the humanized monoclonal anti-amyloid beta (Aβ) antibody is crenezumab.

Embodiment 110. A kit comprising a humanized monoclonal anti-amyloid beta (Aβ) antibody for treating a human patient in need thereof having a genetic mutation that causes familial Alzheimer's Disease (AD), according to the method of any one of embodiments 1-109.

Embodiment 111. A humanized monoclonal anti-amyloid beta (Aβ) antibody for use for treating a human patient in need thereof having a genetic mutation that causes familial Alzheimer's Disease (AD) according to the method of any one of embodiments 1-109.

Embodiment 112. The method of any one of embodiments 1-109, wherein administering such treatment to the plurality of human patients results in an effect on a plasma biomarker compared to a reference plasma biomarker,
wherein the reference plasma biomarker is the plasma biomarker of a plurality of human patients who have received the placebo.

Embodiment 113. The method of embodiment 112, wherein the plasma biomarker is measured using an immunoassay.

Embodiment 114. The method of embodiment 112 or 113, wherein the plasma biomarker is any one of Aβ42, Aβ40, pTau181, pTau217, NfL, GFAP, YKL-40, or sTREM2.

Embodiment 115. The method of embodiment 114, wherein the plasma biomarker is the ratio of Aβ42 to Aβ40.

Embodiment 116. The method of any one of embodiments 112-114, wherein administering such treatment results in an increase of annualized rate of change in the plasma Aβ42 biomarker in the plurality of human patients compared to a reference annualized rate of change in the plasma Aβ42 biomarker, wherein the reference plasma Aβ42 biomarker is an annualized rate of change in the plasma Aβ42 biomarker of a plurality of human patients who have received the placebo.

Embodiment 117. The method of any one of embodiments 112-114, wherein administering such treatment results in an increase of annualized rate of change in the plasma Aβ40 biomarker in the plurality of human patients compared to a reference annualized rate of change in the plasma Aβ40 biomarker, wherein the reference plasma Aβ40 biomarker is an annualized rate of change in the plasma Aβ40 biomarker of a plurality of human patients who have received the placebo.

Embodiment 118. The method of any one of embodiments 112-114, wherein administering such treatment results in a reduction of annualized rate of change in the plasma pTau181 biomarker in the plurality of human patients compared to a reference annualized rate of change in the plasma pTau181 biomarker, wherein the reference plasma pTau181 biomarker is an annualized rate of change in the plasma pTau181 biomarker of a plurality of human patients who have received the placebo.

Embodiment 119. The method of embodiment 118, wherein administering such treatment results in a reduction of annualized rate of change in the plasma pTau181 biomarker in the plurality of human patients by about 6% relative to the reference plasma pTau181 biomarker.

Embodiment 120. The method of any one of embodiments 112-114, wherein administering such treatment results in a reduction of annualized rate of change in the plasma pTau217 biomarker in the plurality of human patients compared to a reference annualized rate of change in the plasma pTau217 biomarker, wherein the reference plasma pTau217 biomarker is an annualized rate of change in the plasma pTau217 biomarker of a plurality of human patients who have received the placebo.

Embodiment 121. The method of embodiment 120, wherein administering such treatment results in a reduction of annualized rate of change in the plasma pTau217 biomarker in the plurality of human patients by about 9% relative to the reference plasma pTau217 biomarker.

Embodiment 122. The method of any one of embodiments 112-114, wherein administering such treatment results in a reduction of annualized rate of change in the plasma neurofilament light (NfL) biomarker in the plurality of human patients compared to a reference annualized rate of change in the plasma NfL biomarker, wherein the reference plasma NfL biomarker is an annualized rate of change in the plasma NfL biomarker of a plurality of human patients who have received the placebo.

Embodiment 123. The method of embodiment 122, wherein administering such treatment results in a reduction of annualized rate of change in the plasma NfL biomarker in the plurality of human patients by about 10% relative to the reference plasma NfL biomarker.

Embodiment 124. The method of any one of embodiments 112-114, wherein administering such treatment results in a reduction of annualized rate of change in the plasma GFAP biomarker in the plurality of human patients compared to a reference annualized rate of change in the plasma GFAP biomarker, wherein the reference plasma GFAP biomarker is an annualized rate of change in the plasma GFAP biomarker of a plurality of human patients who have received the placebo.

Embodiment 125. The method of embodiment 124, wherein administering such treatment results in a reduction of annualized rate of change in the plasma GFAP biomarker in the plurality of human patients by about 17% relative to the reference plasma GFAP biomarker.

Embodiment 126. The method of any one of embodiments 112-114, wherein administering such treatment results in a reduction of annualized rate of change in the plasma YKL-40 biomarker in the plurality of human patients compared to a reference annualized rate of change in the plasma YKL-40 biomarker, wherein the reference plasma YKL-40 biomarker is an annualized rate of change in the plasma YKL-40 biomarker of a plurality of human patients who have received the placebo.

Embodiment 127. The method of embodiment 126, wherein administering such treatment results in a reduction of annualized rate of change in the plasma YKL-40 biomarker in the plurality of human patients by about 12% relative to the reference plasma YKL-40 biomarker.

Embodiment 128. The method of any one of embodiments 112-114, wherein administering such treatment results in a reduction of annualized rate of change in the plasma sTREM2 biomarker in the plurality of human patients compared to a reference annualized rate of change in the plasma sTREM2 biomarker, wherein the reference plasma sTREM2 biomarker is an annualized rate of change in the plasma sTREM2 biomarker of a plurality of human patients who have received the placebo.

Embodiment 129. The method of embodiment 126, wherein administering such treatment results in a reduction of annualized rate of change in the plasma sTREM2 biomarker in the plurality of human patients by about 23% relative to the reference plasma sTREM2 biomarker.

Embodiment 130. The method of any one of embodiments 1-129, wherein administering such treatment results in an increase in hippocampus as measured by vMRI by at least 9%, optionally by about 9.4%.

Embodiment 131. The method of any one of embodiments 1-130, wherein administering such treatment results in a decrease in ventricles as measured by vMRI by at least 2%, optionally by about 2.5%.

Embodiment 132. The method of any one of embodiments 1-131, wherein administering such treatment results in an increase in whole brain as measured by vMRI by at least 12%, optionally by about 13%.

1. A method of delaying onset of at least one symptom in a human patient with a genetic mutation that causes familial Alzheimer's Disease (AD) comprising administering to the human patient an effective amount of a humanized monoclonal anti-amyloid beta (Aβ) antibody,
wherein administering such treatment to a plurality of human patients results in a delayed onset of at least one symptom in the plurality of human patients compared to a reference onset of at least one symptom,
wherein the reference onset of at least one symptom is of a plurality of human patients who have received a placebo,
wherein the antibody comprises six hypervariable regions (HVRs), wherein
(i) HVR-H1 comprises the amino acid sequence set forth in SEQ ID NO:2;
(ii) HVR-H2 comprises the amino acid sequence set forth in SEQ ID NO:3;
(iii) HVR-H3 comprises the amino acid sequence set forth in SEQ ID NO:4;
(iv) HVR-L1 comprises the amino acid sequence set forth in SEQ ID NO:6;
(v) HVR-L2 comprises the amino acid sequence set forth in SEQ ID NO:7; and
(vi) HVR-L3 comprises the amino acid sequence set forth in SEQ ID NO:8.

2. A method of slowing cognitive decline in a human patient with a genetic mutation that causes familial Alzheimer's Disease (AD) comprising administering to the human patient an effective amount of a humanized monoclonal anti-amyloid beta (Aβ) antibody,
wherein administering such treatment to a plurality of human patients results in a delayed cognitive decline in the plurality of human patients compared to a reference cognitive decline,
wherein the reference cognitive decline is of a plurality of human patients who have received a placebo,
wherein the antibody comprises six hypervariable regions (HVRs), wherein
(i) HVR-H1 comprises the amino acid sequence set forth in SEQ ID NO:2;
(ii) HVR-H2 comprises the amino acid sequence set forth in SEQ ID NO:3;
(iii) HVR-H3 comprises the amino acid sequence set forth in SEQ ID NO:4;
(iv) HVR-L1 comprises the amino acid sequence set forth in SEQ ID NO:6;
(v) HVR-L2 comprises the amino acid sequence set forth in SEQ ID NO:7; and
(vi) HVR-L3 comprises the amino acid sequence set forth in SEQ ID NO:8.

3. A method of preventing cognitive impairment in a human patient with a genetic mutation that causes familial Alzheimer's Disease (AD) comprising administering to the human patient an effective amount of a humanized monoclonal anti-amyloid beta (Aβ) antibody,
wherein administering such treatment to a plurality of human patients results in a reduced cognitive impairment in the plurality of human patients compared to a reference cognitive impairment,
wherein the reference cognitive impairment is of a plurality of human patients who have received a placebo,
wherein the antibody comprises six hypervariable regions (HVRs), wherein
(i) HVR-H1 comprises the amino acid sequence set forth in SEQ ID NO:2;
(ii) HVR-H2 comprises the amino acid sequence set forth in SEQ ID NO:3;
(iii) HVR-H3 comprises the amino acid sequence set forth in SEQ ID NO:4;
(iv) HVR-L1 comprises the amino acid sequence set forth in SEQ ID NO:6;
(v) HVR-L2 comprises the amino acid sequence set forth in SEQ ID NO:7; and
(vi) HVR-L3 comprises the amino acid sequence set forth in SEQ ID NO:8.

4. The method of claim 1, wherein administering such treatment results in a delay in onset of at least one symptom compared to the reference onset of at least one symptom after treatment of about five years or longer.

5. The method of claim 2, wherein administering such treatment results in a slowing of cognitive decline in the plurality of human patients compared to the reference cognitive decline after treatment of about five years or longer.

6. The method of claim 3, wherein administering such treatment results in a prevention or reduction of cognitive impairment in the plurality of human patients compared to the reference cognitive impairment after treatment of about five years or longer.

7. The method of any one of claims 1-6,
wherein the delay in onset of at least one symptom, the slowing in cognitive decline, or the prevention of cognitive impairment is measured using an API ADAD Cognitive Composite Test Battery,
wherein, in the API ADAD Cognitive Composite Test Battery, administering such treatment to the plurality of human patients results in a reduced annualized rate of change on an API ADAD Composite score of the plurality of human patients relative to a reference annualized rate of change on an API ADAD Composite score, and
wherein the reference annualized rate of change on an API ADAD Composite score is an annualized rate of change on an API ADAD Composite score of a plurality of human patients who have received the placebo.

8. The method of claim 7, wherein the API ADAD Composite Cognitive Test Battery comprises a Word List Recall, Multilingual Naming Test, Mini-Mental State Examination (MMSE), CERAD Constructional praxis, and Raven's Progressive Matrices.

9. The method of any one of claims 7-8, wherein administering such treatment results in a reduced annualized rate of change in the API ADAD Composite score after treatment of about five years or longer.

10. The method of any one of claims 7-9, wherein administering such treatment results in a reduction of the annualized rate of change on the API ADAD Composite score in the plurality of human patients by at least 20% relative to the reference annualized rate of change on the API ADAD Composite score.

11. The method of any one of claims 1-10, wherein administering such treatment to the plurality of human patients results in a reduced annualized rate of change on a Free and Cued Selective Reminding Task (FCSRT) Cueing Index of the plurality of human patients relative to a reference annualized rate of change on a FCSRT Cueing Index,
wherein the reference annualized rate of change on the FCSRT Cueing Index is an annualized rate of change on a FCSRT Cueing Index of a plurality of human patients who have received the placebo.

12. The method of claim 11, wherein administering such treatment results in a reduced annualized rate of change on the FCSRT Cueing Index in the plurality of human patients compared to the reference annualized rate of change on the FCSRT Cueing Index after treatment of about five years or longer.

13. The method of any one of claims 11-12, wherein administering such treatment results in a reduction of the annualized rate of change of the FCSRT Cueing Index in the plurality of human patients by at least 10% relative to the reference annualized rate of change on the FCSRT Cueing Index.

14. The method of any one of claims 11-13, wherein the FCSRT Cueing Index is assessed using controlled learning.

15. The method of any one of claims 1-14, wherein administering such treatment to the plurality of human patients results in an increased time to progression from preclinical AD to mild cognitive impairment due to AD or from preclinical AD to dementia due to AD in the plurality of human patients relative to a reference time to progression from preclinical AD to mild cognitive impairment due to AD or from preclinical AD to dementia due to AD,
wherein the reference time to progression from preclinical AD to mild cognitive impairment due to AD or from preclinical AD to dementia due to AD is a time to progression of a plurality of human patients who have received the placebo.

16. The method of claim 15, wherein administering such treatment results in an increased time to progression from preclinical AD to mild cognitive impairment due to AD or from preclinical AD to dementia due to AD in the plurality of human patients relative to the reference time to progression from preclinical AD to mild cognitive impairment due to AD or from preclinical AD to dementia due to AD after treatment of about five years or longer.

17. The method of any one of claims 15-16, wherein administering such treatment results in an increase of the time to progression from preclinical AD to mild cognitive impairment due to AD or from preclinical AD to dementia due to AD in the plurality of human patients by at least 10% as compared to a reference time to progression from preclinical AD to mild cognitive impairment due to AD or from preclinical AD to dementia due to AD.

18. The method of any one of claims 1-17, wherein administering such treatment to the plurality of human patients results in an increased time to progression to non-zero in the Clinical Dementia Rating (CDR) Scale global score of the plurality of human patients relative to a reference time to progression to non-zero in the CDR Scale global score, wherein the reference time to progression to non-zero in the CDR Scale global score is a time to progression of a plurality of human patients who have received the placebo.

19. The method of claim 18, wherein administering such treatment results in an increased time to progression to non-zero in the CDR Scale global score in the plurality of human patients by at least 5% relative to a reference time to progression to progression to non-zero in the CDR Scale global score.

20. The method of any one of claims 1-19, wherein administering such treatment to the plurality of human patients results in a reduced annualized rate of change on a Clinical Dementia Rating (CDR) Scale Sum of Boxes of the plurality of human patients relative to a reference annualized rate of change on a CDR Scale Sum of Boxes,
wherein the reference annualized rate of change on a CDR Scale Sum of Boxes is an annualized rate of change on a CDR Scale Sum of Boxes of a plurality of human patients who have received placebo.

21. The method of claim 20, wherein administering such treatment results in a reduced annualized rate of change on a CDR Scale Sum of Boxes of the plurality of human patients compared to the reference annualized rate of change on a CDR Scale Sum of Boxes after treatment of about five years or longer.

22. The method of any one of claims 18-21, wherein administering such treatment results in a reduced annualized rate of change in a CDR Scale Sum of Boxes global score in the plurality of human patients by at least 5% relative to a reference CDR Scale Sum of Boxes global score.

23. The method of any one of claims 1-22, wherein administering such treatment to the plurality of human patients results in a reduced annualized rate of change in a measure of overall neurocognitive functioning of the plurality of human patients relative to a reference annualized rate of change in a measure of overall neurocognitive functioning
wherein the reference annualized rate of change in a measure of overall neurocognitive functioning is an annualized rate of change in a measure of overall neurocognitive functioning of the plurality of human patients who have received placebo.

24. The method of claim 23, wherein the annualized rate of change in a measure of overall neurocognitive functioning is determined using a Repeatable Battery for the Assessment of Neuropsychological Status (RBANS) score.

25. The method of claim 24, wherein administering such treatment results in a reduction of an annualized rate of change of a RBANS score in the plurality of human patients compared to a reference annualized rate of change of a RBANS score, wherein the reference annualized rate of change of a RBANS score is an annualized rate of change of a RBANS score of a plurality of human patients who have received placebo.

26. The method of any one of claims 24-25, wherein administering such treatment results in a reduction of the annualized rate of change of a RBANS score in the plurality of human patients compared to the reference annualized rate of change of a RBANS score after treatment of about 5 years or longer.

27. The method of any one of claims 24-26, wherein administering such treatment results in a reduction of the RBANS score by at least 4% relative to the reference RBANS score.

28. The method of any one of claims 1-27, wherein administering such treatment to the plurality of human patients results in an effect on a tau-based CSF biomarker compared to a reference tau-based CSF biomarker,
wherein the reference tau-based CSF biomarker is the tau-based CSF biomarker of a plurality of human patients who have received the placebo.

29. The method of claim 28, wherein the tau-based CSF biomarker is measured using positron emission tomography.

30. The method of any one of claims 28-29, wherein administering such treatment results in a reduction of annualized rate of change in the tau-based CSF biomarker in the plurality of human patients compared to a reference annualized rate of change in the tau-based CSF biomarker, wherein the reference tau-based CSF biomarker is an annualized rate of change in the tau-based CSF biomarker of a plurality of human patients who have received the placebo.

31. The method of claim 30, wherein administering such treatment results in a reduction of annualized rate of the tau-based CSF biomarker in the plurality of human patients by at least 30% relative to the reference tau-based CSF biomarker, said tau-based CSF biomarker being a phospho-tau-based CSF biomarker.

32. The method of claim 30, wherein administering such treatment results in a reduction of annualized rate of the tau-based CSF biomarker in the plurality of human patients by at least 20% relative to the reference tau-based CSF biomarker, said tau-based CSF biomarker being a total-tau-based CSF biomarker.

33. The method of any one of claims 1-32, wherein administering such treatment to the plurality of human patients results in an effect on a brain tau load compared to a reference a brain tau load,
wherein the reference brain tau load is a brain tau load of a plurality of human patients who have received the placebo.

34. The method of claim 33, wherein administering such treatment results in a reduction of annualized rate of the tau-PET in the plurality of human patients by at least 50% relative to the reference tau-PET.

35. The method of any one of claims 1-34, wherein administering such treatment to the plurality of human patients results in a reduction of cerebral fibrillary amyloid burden in a predefined region of interest of the plurality of human patients relative to a reference cerebral fibrillary amyloid burden in a predefined region of interest,
wherein the reference cerebral fibrillary amyloid burden in a predefined region of interest is a cerebral fibrillary amyloid burden in a predefined region of interest of a plurality of human patients who have received the placebo.

36. The method of claim 35, wherein administering such treatment results in a reduction of annualized rate of change in amyloid burden measured by PET in the plurality of human patients by 3% relative to a reference amyloid burden measured by PET.

37. The method of any one of claims 1-36, wherein administering such treatment to the plurality of human patients results in a reduced decline in regional cerebral metabolic rate of glucose (CMRgI) of the plurality of human patients relative to a reference CMRgI,
wherein the reference CMRgI is a CMRgI of the plurality of human patients who have received the placebo.

38. The method of claim 37, wherein administering such treatment results in a reduced FDG PET measurement in the plurality of human patients relative to a reference FDG PET measurement, wherein the reference FDG PET measurement is a FDG PET measurement of the plurality of human patients who have received the placebo.

39. The method of claim 38, wherein administering such treatment results in a reduced annualized Standardized Uptake Value Ratio (SUVR) of FDG PET measurement in the plurality of human patients as compared to a reference annualized SUVR of FDG PET, wherein the reference annualized SUVR of FDG PET is an annualized SUVR of FDG PET of the plurality of human patients who have received the placebo.

40. The method of claim 39, wherein administering such treatment results in a reduced annualized Standardized Uptake Value Ratio (SUVR) of FDG PET measurement in the plurality of human patients by at least 10% as compared to a reference annualized SUVR of FDG PET,
wherein the reference brain atrophy is a brain atrophy of the plurality of human patients who have received the placebo.

41. The method of any one of claims 1-40, wherein administering such treatment to the plurality of human patients results in a reduced annualized rate of change in the brain atrophy of the plurality of human patients relative to a reference annualized rate of change in the brain atrophy,
wherein the reference annualized rate of change in the brain atrophy is an annualized rate of change in the brain atrophy of the plurality of human patients who have received the placebo.

42. The method of claim 41, wherein administering such treatment results in a reduction of an annualized rate of change in the brain atrophy of the plurality of human patients compared to the reference annualized rate of change in the brain atrophy after treatment of about five years or longer.

43. The method of claim 42, wherein administering such treatment results in a reduction of an annualized rate of change in the brain atrophy of the plurality of human patients by 5% to 20% relative to the reference brain atrophy, said reduction being measured by a volumetric MRI on a whole brain.

44. The method of claim 43, wherein the volumetric MRI is measured in a bilateral hippocampus.

45. The method of claim 44, wherein administering such treatment results in a reduction of an annualized rate of change in the brain atrophy of the plurality of human patients by at least 1% relative to the reference brain atrophy, said reduction being measured by a volumetric MRI on a bilateral hippocampus.

46. The method of any one of claims 1-45, wherein administering such treatment results in a reduction in change over baseline in a cognitive measurement of the plurality of human patients compared to a reference cognitive measurement,
wherein the reference cognitive measurement is a cognitive measurement of a plurality of human patients who have received the placebo,
wherein the cognitive measurement is selected from the group consisting of i) Trial Making Test, ii) Mini-Mental State Examination (MMSE), iii) Repeatable Battery for the Assessment of Neuropsychological Status (RBANS) Index Scores, iv) scores of each of the components of the API ADAD Composite Cognitive Test Battery, v) Preclinical Alzheimer's Cognitive Composite (PACC), and vi) other clinical endpoints.

47. The method of claim 46, wherein administering such treatment results in a reduction in change over baseline in the cognitive measurement of the plurality of human patients compared to the reference cognitive measurement after treatment of about five years or longer.

48. The method of any one of claims 1-47, wherein administering such treatment results in a reduction in change over baseline in a Neuropsychiatric Inventory (NPI) of the plurality of human patients compared to a reference NPI,
wherein the reference NPI is a NPI of a plurality of human patients who have received the placebo.

49. The method of any one of claims 1-48, wherein administering such treatment results in a reduction in change over baseline in a Geriatric Depression Scale (GDS) of the plurality of human patients compared to a reference GDS,
wherein the reference GDS is a GDS of a plurality of human patients who have received the placebo.

50. The method of any one of claims 1-49, wherein administering such treatment results in a reduction in change over baseline in a Changes in Functional Assessment Staging of Alzheimer's Disease (FAST) total score of the plurality of human patients compared to a reference FAST total score,
wherein the reference FAST total score is the FAST total score of a plurality of human patients who have received the placebo.

51. The method of any one of claims 1-50, wherein administering such treatment results in a reduction in change over baseline in a Changes in Subject Memory Checklist of the plurality of human patients compared to a reference Changes in Subject Memory Checklist,
wherein the reference Changes in Subject Memory Checklist is a Changes in Subject Memory Checklist of a plurality of human patients who have received the placebo.

52. The method of any one of claims 1-51, wherein the genetic mutation that causes familial AD is an autosomal dominant mutation causing Autosomal Dominant Alzheimer's Disease (ADAD).

53. The method of claim 52, wherein the ADAD comprises one or more mutations in one or more genes selected from the group consisting of presenilin 1 (PSEN1), presenilin 2 (PSEN2), and/or amyloid precursor protein (APP).

54. The method of any one of claims 1-53, wherein the humanized monoclonal anti-amyloid beta (Aβ) antibody is delivered intravenously.

55. The method of claim 54, wherein the humanized monoclonal anti-amyloid beta (Aβ) antibody is administered at a) a dose of about 60 mg/kg or higher; or b) a fixed dose of 4200 mg or higher; or c) a fixed dose of about 4200 mg.

56. The method of any one of claims 54-55, wherein the humanized monoclonal anti-amyloid beta (Aβ) antibody is delivered about every four weeks (Q4W).

57. The method of any one of claims 54-55, wherein the humanized monoclonal anti-amyloid beta (Aβ) antibody is delivered about Q4W for about 5 years.

58. The method of any one of claim 1-53 or 55-57, wherein the humanized monoclonal anti-amyloid beta (Aβ) antibody is delivered subcutaneously.

59. The method of claim 58, wherein the humanized monoclonal anti-amyloid beta (Aβ) antibody is delivered at a dose of about 720 mg or higher.

60. The method of claim 58, wherein the humanized monoclonal anti-amyloid beta (Aβ) antibody is delivered at a dose of about 300 mg.

61. The method of any one of claims 58-60, wherein, the humanized monoclonal anti-amyloid beta (Aβ) antibody is delivered every other week (Q2W).

62. The method of any one of claims 58-61, wherein the humanized monoclonal anti-amyloid beta (Aβ) antibody is delivered about Q2W for about 5 years.

63. The method of any one of claims 1-62, wherein the humanized monoclonal anti-amyloid beta (Aβ) antibody comprises a heavy chain variable region (VH) comprising the amino acid sequence of SEQ ID NO: 10 and a light chain variable region (VL) comprising the amino acid sequence of SEQ ID NO:11.

64. The method of any one of claims 1-63, wherein the humanized monoclonal anti-amyloid beta (Aβ) antibody comprises a heavy chain comprising the amino acid sequence of SEQ ID NO: 5 and a light chain comprising the amino acid sequence of SEQ ID NO:9.

65. The method of any one of claims 1-64, wherein administering such treatment results in a reduced Standardized Uptake Value Ratio (SUVR) of amyloid PET measurement in the plurality of human patients as compared to a reference SUVR of amyloid PET, wherein the reference SUVR of amyloid PET is a SUVR of amyloid PET of the plurality of human patients who have received the placebo.

66. The method of claim 65, wherein administering such treatment results in a reduced Standardized Uptake Value Ratio (SUVR) of amyloid PET measurement in the plurality of human patients by at least 3% as compared to the reference SUVR of amyloid PET.

67. The method of claim 65, wherein administering such treatment results in a reduction of cerebrospinal fluid (CSF) neurofilament light (CSF NfL) in the plurality of human patients as compared to a reference CSF NfL, wherein the reference CSF NfL is from the plurality of human patients who have received the placebo.

68. The method of claim 67, wherein administering such treatment results in a relative reduction of CSF NfL in the plurality of human patients of at least 10% relative to the reference CSF NfL.

69. The method of any one of claims 1-68, wherein the humanized monoclonal anti-amyloid beta (Aβ) antibody is crenezumab.

70. A kit comprising a humanized monoclonal anti-amyloid beta (Aβ) antibody for treating a human patient in need thereof having a genetic mutation that causes familial Alzheimer's Disease (AD), according to the method of any one of claims 1-69.

71. A humanized monoclonal anti-amyloid beta (Aβ) antibody for use for treating a human patient in need thereof having a genetic mutation that causes familial Alzheimer's Disease (AD) according to the method of any one of claims 1-69.
